SEQUENCE LISTING (263 sequences)

SEQ ID NO: 1 - protein, 25 aa, synthetic construct
  REGION 1..25: note = Synthetic
EVQLVESGGGLVQPGRSLRLSCTAS

SEQ ID NO: 2 - protein, 25 aa, synthetic construct
  REGION 1..25: note = Synthetic
EVQLVESGGGLVKPGGSLRLSCAAS

SEQ ID NO: 3 - protein, 25 aa, synthetic construct
  REGION 1..25: note = Synthetic
EVQLVESGGGLVKPGGSLKLSCAAS

SEQ ID NO: 4 - protein, 25 aa, synthetic construct
  REGION 1..25: note = Synthetic
EVQLVESGGGLVKPGGSLRLSCVAS

SEQ ID NO: 5 - protein, 25 aa, synthetic construct
  REGION 1..25: note = Synthetic
EVQLVESGGGLVKPGGSLRLSCVTS

SEQ ID NO: 6 - protein, 25 aa, synthetic construct
  REGION 1..25: note = Synthetic
QVQLVESGGGLVKPGGSLRLSCAAS

SEQ ID NO: 7 - protein, 25 aa, synthetic construct
  REGION 1..25: note = Synthetic
EVQLLESGGGLVQPGGSLRLSCAAS

SEQ ID NO: 8 - protein, 25 aa, synthetic construct
  REGION 1..25: note = Synthetic
QVQLVESGGGLVKPGGSLRISCAAS

SEQ ID NO: 9 - protein, 25 aa, synthetic construct [sequence omitted]

SEQ ID NO: 10 - protein, 7 aa, synthetic construct
  REGION 1..7: note = Synthetic
GSDINDY

SEQ ID NO: 11 - protein, 7 aa, synthetic construct
  REGION 1..7: note = Synthetic
GFIFSDY

SEQ ID NO: 12 - protein, 7 aa, synthetic construct
  REGION 1..7: note = Synthetic
GFTFSDY

SEQ ID NO: 13 - protein, 7 aa, synthetic construct
  REGION 1..7: note = Synthetic
GFNFSDY

SEQ ID NO: 14 - protein, 7 aa, synthetic construct
  REGION 1..7: note = Synthetic
GFTLSDY

SEQ ID NO: 15 - protein, 7 aa, synthetic construct
  REGION 1..7: note = Synthetic
GFTLSDS

SEQ ID NO: 16 - protein, 7 aa, synthetic construct
  REGION 1..7: note = Synthetic
GFKLSDF

SEQ ID NO: 17 - protein, 7 aa, synthetic construct
  REGION 1..7: note = Synthetic
GFTFSDF

SEQ ID NO: 18 - protein, 7 aa, synthetic construct
  REGION 1..7: note = Synthetic
GFTFSSH

SEQ ID NO: 19 - protein, 7 aa, synthetic construct
  REGION 1..7: note = Synthetic
GFTFSNY

SEQ ID NO: 20 - protein, 7 aa, synthetic construct
  REGION 1..7: note = Synthetic
GFTFSNF

SEQ ID NO: 21 - protein, 7 aa, synthetic construct
  REGION 1..7: note = Synthetic
GFNLSDS

SEQ ID NO: 22 - protein, 7 aa, synthetic construct
  REGION 1..7: note = Synthetic
GFIFGSY

SEQ ID NO: 23 - protein, 7 aa, synthetic construct
  REGION 1..7: note = Synthetic
GFTFSGN

SEQ ID NO: 24 - protein, 19 aa, synthetic construct
  REGION 1..19: note = Synthetic
PITWVRQAPGQGLEWIGFI

SEQ ID NO: 25 - protein, 19 aa, synthetic construct
  REGION 1..19: note = Synthetic
YMSWIRQAPGKGLEWVSYI

SEQ ID NO: 26 - protein, 19 aa, synthetic construct
  REGION 1..19: note = Synthetic
YMSWIRQAPGKGLEWISYI

SEQ ID NO: 27 - protein, 19 aa, synthetic construct
  REGION 1..19: note = Synthetic
YMSWIRQAPGKGVEWISYI

SEQ ID NO: 28 - protein, 19 aa, synthetic construct
  REGION 1..19: note = Synthetic
YMSWIRQAPGKGMEWISYI

SEQ ID NO: 29 - protein, 19 aa, synthetic construct
  REGION 1..19: note = Synthetic
YMSWIRQAPGKGLEWIAYI

SEQ ID NO: 30 - protein, 19 aa, synthetic construct
  REGION 1..19: note = Synthetic
YMSWIRQAPGKGLEWVSNI

SEQ ID NO: 31 - protein, 19 aa, synthetic construct
  REGION 1..19: note = Synthetic
YMSWIRQTPGKGLEWVSYI

SEQ ID NO: 32 - protein, 19 aa, synthetic construct
  REGION 1..19: note = Synthetic
QMSWIRQAPGKGVEWVSYI

SEQ ID NO: 33 - protein, 19 aa, synthetic construct
  REGION 1..19: note = Synthetic
QMSWIRQAPGKGLEWVAYI

SEQ ID NO: 34 - protein, 19 aa, synthetic construct
  REGION 1..19: note = Synthetic
AMSWVRQAPGKGLEWVSAI

SEQ ID NO: 35 - protein, 19 aa, synthetic construct
  REGION 1..19: note = Synthetic
NMSWIRQAPGKGVEWVSHI

SEQ ID NO: 36 - protein, 19 aa, synthetic construct
  REGION 1..19: note = Synthetic
WMYWVRQAPGKGLVWVSRI

SEQ ID NO: 37 - protein, 19 aa, synthetic construct
  REGION 1..19: note = Synthetic
YMSWIRQAPGKGLEWVSCI

SEQ ID NO: 38 - protein, 19 aa, synthetic construct
  REGION 1..19: note = Synthetic
AMSWVRQAPGKGLEWVSGI

SEQ ID NO: 39 - protein, 5 aa, synthetic construct
  REGION 1..5: note = Synthetic
NSGGS

SEQ ID NO: 40 - protein, 6 aa, synthetic construct
  REGION 1..6: note = Synthetic
SSSGST

SEQ ID NO: 41 - protein, 6 aa, synthetic construct
  REGION 1..6: note = Synthetic
SSSGTT

SEQ ID NO: 42 - protein, 6 aa, synthetic construct
  REGION 1..6: note = Synthetic
SNNGST

SEQ ID NO: 43 - protein, 6 aa, synthetic construct
  REGION 1..6: note = Synthetic
SSSGSS

SEQ ID NO: 44 - protein, 6 aa, synthetic construct
  REGION 1..6: note = Synthetic
SNSSST

SEQ ID NO: 45 - protein, 6 aa, synthetic construct
  REGION 1..6: note = Synthetic
SNSSSS

SEQ ID NO: 46 - protein, 6 aa, synthetic construct
  REGION 1..6: note = Synthetic
SKNGFT

SEQ ID NO: 47 - protein, 6 aa, synthetic construct
  REGION 1..6: note = Synthetic
STTGST

SEQ ID NO: 48 - protein, 6 aa, synthetic construct
  REGION 1..6: note = Synthetic
SRSGST

SEQ ID NO: 49 - protein, 6 aa, synthetic construct
  REGION 1..6: note = Synthetic
SSSGNT

SEQ ID NO: 50 - protein, 6 aa, synthetic construct
  REGION 1..6: note = Synthetic
DTNGST

SEQ ID NO: 51 - protein, 6 aa, synthetic construct
  REGION 1..6: note = Synthetic
SSSGGS

SEQ ID NO: 52 - protein, 6 aa, synthetic construct
  REGION 1..6: note = Synthetic
SSSGSY

SEQ ID NO: 53 - protein, 6 aa, synthetic construct
  REGION 1..6: note = Synthetic
SSSGSW

SEQ ID NO: 54 - protein, 6 aa, synthetic construct
  REGION 1..6: note = Synthetic
NTRGSP

SEQ ID NO: 55 - protein, 6 aa, synthetic construct
  REGION 1..6: note = Synthetic
DSSGRP

SEQ ID NO: 56 - protein, 6 aa, synthetic construct
  REGION 1..6: note = Synthetic
SGSGDS

SEQ ID NO: 57 - protein, 6 aa, synthetic construct
  REGION 1..6: note = Synthetic
SGSGRT

SEQ ID NO: 58 - protein, 6 aa, synthetic construct
  REGION 1..6: note = Synthetic
NSDGSS

SEQ ID NO: 59 - protein, 6 aa, synthetic construct
  REGION 1..6: note = Synthetic
SSNGST

SEQ ID NO: 60 - protein, 6 aa, synthetic construct
  REGION 1..6: note = Synthetic
SGTGGN

SEQ ID NO: 61 - protein, 6 aa, synthetic construct
  REGION 1..6: note = Synthetic
SGSGDK

SEQ ID NO: 62 - protein, 41 aa, synthetic construct
  REGION 1..41: note = Synthetic
TWYASWVKGRFTISRDDSKSIAYLQMNSLKTEDTAVYYCAR

SEQ ID NO: 63 - protein, 41 aa, synthetic construct
  REGION 1..41: note = Synthetic
IHYADSVKGRFTVSRDNAKNSLYLQMNSLRTEDTAVYYCAR

SEQ ID NO: 64 - protein, 41 aa, synthetic construct
  REGION 1..41: note = Synthetic
IHYADSVKGRFTISRDNAKNSLYLQMNSLRTEDTAVYYCAR

SEQ ID NO: 65 - protein, 41 aa, synthetic construct
  REGION 1..41: note = Synthetic
IHYADSVKGRFTISRDNAKNSLYLQMNSLRAEDTAVYYCAR

SEQ ID NO: 66 - protein, 41 aa, synthetic construct
  REGION 1..41: note = Synthetic
IHYADSVKGRFTISRDNAKNSLYLQMNSLRAEDTAIYYCAR

SEQ ID NO: 67 - protein, 41 aa, synthetic construct
  REGION 1..41: note = Synthetic
IYYAESVKGRFTISRDNAKNSLYLQMNRLRAEDTALYYCAR

SEQ ID NO: 68 - protein, 41 aa, synthetic construct
  REGION 1..41: note = Synthetic
IYYADSVKGRFTISRDNAKNSLYLQMSRLRAEDTAMYYCAR

SEQ ID NO: 69 - protein, 41 aa, synthetic construct
  REGION 1..41: note = Synthetic
KNYANSVKGRFTISRDNAKNSLYLQMNSLRAEDTAAYYCAR

SEQ ID NO: 70 - protein, 41 aa, synthetic construct
  REGION 1..41: note = Synthetic
ISYANSVKGRFTVSRDNAKNSLYLQMNSLRAEDTALYYCAR

SEQ ID NO: 71 - protein, 41 aa, synthetic construct
  REGION 1..41: note = Synthetic
IHYSDSVKGRFTISRDNAKNSLYLQMNSLRAEDTAVYYCAR

SEQ ID NO: 72 - protein, 41 aa, synthetic construct
  REGION 1..41: note = Synthetic
IYYAESVKGRFTVSRDNAKNSLYLQMNSLRAEDTAIYYCAR

SEQ ID NO: 73 - protein, 41 aa, synthetic construct
  REGION 1..41: note = Synthetic
KNYANSVKGRFTISRDNAKSSLYLQMNSLRAEDTAAYYCAR

SEQ ID NO: 74 - protein, 41 aa, synthetic construct
  REGION 1..41: note = Synthetic
KYYADSVKGRFTISRDNAKNSLYLQMNSLRAEDTAVYYCAR

SEQ ID NO: 75 - protein, 41 aa, synthetic construct
  REGION 1..41: note = Synthetic
IYYGDSVKGRFTISRDNAKNSLFLQMNSLRAEDTAVYYCAR

SEQ ID NO: 76 - protein, 41 aa, synthetic construct
  REGION 1..41: note = Synthetic
RYYAESVKGRFTLSRDNVKNSLNLQMNGLRAEDTALYYCAR

SEQ ID NO: 77 - protein, 41 aa, synthetic construct
  REGION 1..41: note = Synthetic
IYYADSVKGRFTISRDNAKNSLYLQMNRLRAEDTALYYCAR

SEQ ID NO: 78 - protein, 41 aa, synthetic construct
  REGION 1..41: note = Synthetic
IGYADSVKGRFTISRDNAKNSLYLQMNSLRAEDTAIYYCAR

SEQ ID NO: 79 - protein, 41 aa, synthetic construct
  REGION 1..41: note = Synthetic
RYYADSVKGRFTISRDNAKNSLYLQMNSLRAEDTAIYYCAR

SEQ ID NO: 80 - protein, 41 aa, synthetic construct
  REGION 1..41: note = Synthetic
LAYADSVKGRFTISRDNAKNSLYLQMNSLRAEDTAIYYCAR

SEQ ID NO: 81 - protein, 41 aa, synthetic construct
  REGION 1..41: note = Synthetic
IHYAHSVKGRFTISRDNAKNSLYLQMNSLRAEDTAIYYCAR

SEQ ID NO: 82 - protein, 41 aa, synthetic construct
  REGION 1..41: note = Synthetic
IHYSGSVKGRFTISRDNAKNSLYLQMNSLRAEDTAIYYCAR

SEQ ID NO: 83 - protein, 41 aa, synthetic construct
  REGION 1..41: note = Synthetic
IHYSHSVKGRFTISRDNAKNSLYLQMNSLRAEDTAIYYCAR

SEQ ID NO: 84 - protein, 41 aa, synthetic construct
  REGION 1..41: note = Synthetic
IHYSSSVKGRFTISRDNAKNSLYLQMNSLRAEDTAIYYCAR

SEQ ID NO: 85 - protein, 41 aa, synthetic construct
  REGION 1..41: note = Synthetic
IHYSPSVKGRFTISRDNAKNSLYLQMNSLRAEDTAIYYCAR

SEQ ID NO: 86 - protein, 41 aa, synthetic construct
  REGION 1..41: note = Synthetic
IHYSASVKGRFTISRDNAKNSLYLQMNSLRAEDTAIYYCAR

SEQ ID NO: 87 - protein, 41 aa, synthetic construct
  REGION 1..41: note = Synthetic
IHYSTSVKGRFTISRDNAKNSLYLQMNSLRAEDTAIYYCAR

SEQ ID NO: 88 - protein, 41 aa, synthetic construct
  REGION 1..41: note = Synthetic
IHYAGSVKGRFTISRDNAKNSLYLQMNSLRAEDTAIYYCAR

SEQ ID NO: 89 - protein, 41 aa, synthetic construct
  REGION 1..41: note = Synthetic
IHYATSVKGRFTISRDNAKNSLYLQMNSLRAEDTAIYYCAR

SEQ ID NO: 90 - protein, 41 aa, synthetic construct
  REGION 1..41: note = Synthetic
IHYASSVKGRFTISRDNAKNSLYLQMNSLRAEDTAIYYCAR

SEQ ID NO: 91 - protein, 41 aa, synthetic construct
  REGION 1..41: note = Synthetic
THYAASVKGRFTISRDNSKNTLYLQMNSLRAEDTAVYYCET

SEQ ID NO: 92 - protein, 41 aa, synthetic construct
  REGION 1..41: note = Synthetic
IYYAESVKGRFTISRDNAKNSLYLQMNSLRAEDTAMYYCAR

SEQ ID NO: 93 - protein, 41 aa, synthetic construct
  REGION 1..41: note = Synthetic
TNYADSVKGRFTISRDNAKNTLYLQMNSLRVEDTAVYSCAR

SEQ ID NO: 94 - protein, 41 aa, synthetic construct
  REGION 1..41: note = Synthetic
IYYADSVKGRFTISRDNARNSLYLQMNSLRAEDTALYYCAR

SEQ ID NO: 95 - protein, 41 aa, synthetic construct
  REGION 1..41: note = Synthetic
IYYADSVKGRFTVSRDNAKNSLYLQMNHLRAEDTALYYCAR

SEQ ID NO: 96 - protein, 41 aa, synthetic construct
  REGION 1..41: note = Synthetic
TYYADSVKGRFTISRDNSKNTLYLQMNSLRAEDTAVYFCER

SEQ ID NO: 97 - protein, 41 aa, synthetic construct
  REGION 1..41: note = Synthetic
IYYADSVKGRFTISRDNAKNSLYLQMNSLRAEDTAIYYCAR

SEQ ID NO: 98 - protein, 41 aa, synthetic construct
  REGION 1..41: note = Synthetic
TYYAASVKGRFTISRDNSNNTVYLQMNSLRAEDTAVYYCEK

SEQ ID NO: 99 - protein, 11 aa, synthetic construct
  REGION 1..11: note = Synthetic
GYSTYYRDFNI

SEQ ID NO: 100 - protein, 16 aa, synthetic construct
  REGION 1..16: note = Synthetic
DPPTSNSDWVSLHFDH

SEQ ID NO: 101 - protein, 16 aa, synthetic construct
  REGION 1..16: note = Synthetic
DAPSSNSDWVSLQFDY

SEQ ID NO: 102 - protein, 12 aa, synthetic construct
  REGION 1..12: note = Synthetic
DFNNGWYEDFDY

SEQ ID NO: 103 - protein, 16 aa, synthetic construct
  REGION 1..16: note = Synthetic
DIPSSSSDWVSLQFDY

SEQ ID NO: 104 - protein, 14 aa, synthetic construct
  REGION 1..14: note = Synthetic
TPPSSDNWYEDFDY

SEQ ID NO: 105 - protein, 12 aa, synthetic construct
  REGION 1..12: note = Synthetic
DTTNGWYEDFDY

SEQ ID NO: 106 - protein, 14 aa, synthetic construct
  REGION 1..14: note = Synthetic
VPPYNASWYEDFDY

SEQ ID NO: 107 - protein, 12 aa, synthetic construct
  REGION 1..12: note = Synthetic
SPRGAFYEDFDY

SEQ ID NO: 108 - protein, 16 aa, synthetic construct
  REGION 1..16: note = Synthetic
DTPSSSSDWVSLQFDY

SEQ ID NO: 109 - protein, 11 aa, synthetic construct
  REGION 1..11: note = Synthetic
DSSGWYSEFDY

SEQ ID NO: 110 - protein, 14 aa, synthetic construct
  REGION 1..14: note = Synthetic
VPPSNASWYEDFDY

SEQ ID NO: 111 - protein, 16 aa, synthetic construct
  REGION 1..16: note = Synthetic
DAPSSNSDWVSLHFDH

SEQ ID NO: 112 - protein, 12 aa, synthetic construct
  REGION 1..12: note = Synthetic
SPRSAFYEDFDY

SEQ ID NO: 113 - protein, 14 aa, synthetic construct
  REGION 1..14: note = Synthetic
VPPSSSNWYEDFDI

SEQ ID NO: 114 - protein, 14 aa, synthetic construct
  REGION 1..14: note = Synthetic
IPSYTSSWYEDFDH

SEQ ID NO: 115 - protein, 14 aa, synthetic construct
  REGION 1..14: note = Synthetic
TPPSSNNWYEDFDY

SEQ ID NO: 116 - protein, 16 aa, synthetic construct
  REGION 1..16: note = Synthetic
DMPSSSSDWVDLQFDY

SEQ ID NO: 117 - protein, 16 aa, synthetic construct
  REGION 1..16: note = Synthetic
DIPSSSSDWVDLQFDY

SEQ ID NO: 118 - protein, 16 aa, synthetic construct
  REGION 1..16: note = Synthetic
DMPSSSSDWVSLQFDY

SEQ ID NO: 119 - protein, 16 aa, synthetic construct
  REGION 1..16: note = Synthetic
DMPSSSEDWVDLQFDY

SEQ ID NO: 120 - protein, 16 aa, synthetic construct
  REGION 1..16: note = Synthetic
DMPSSSIDWVELQFDY

SEQ ID NO: 121 - protein, 16 aa, synthetic construct
  REGION 1..16: note = Synthetic
DMPSSSKDWVDLQFDY

SEQ ID NO: 122 - protein, 16 aa, synthetic construct
  REGION 1..16: note = Synthetic
DMPSSSIDWVDLQFDY

SEQ ID NO: 123 - protein, 16 aa, synthetic construct
  REGION 1..16: note = Synthetic
DMPSSSEDWVELQFDY

SEQ ID NO: 124 - protein, 16 aa, synthetic construct
  REGION 1..16: note = Synthetic
DMPSSSSDWVELQFDY

SEQ ID NO: 125 - protein, 16 aa, synthetic construct
  REGION 1..16: note = Synthetic
DMPSSSEDWVMLQFDY

SEQ ID NO: 126 - protein, 15 aa, synthetic construct
  REGION 1..15: note = Synthetic
LLRFLESLGNDGFKI

SEQ ID NO: 127 - protein, 12 aa, synthetic construct
  REGION 1..12: note = Synthetic
DLSSGWYEDFDY

SEQ ID NO: 128 - protein, 11 aa, synthetic construct
  REGION 1..11: note = Synthetic
EGSGWYEDFDY

SEQ ID NO: 129 - protein, 12 aa, synthetic construct
  REGION 1..12: note = Synthetic
DVSSGWYEDFDY

SEQ ID NO: 130 - protein, 12 aa, synthetic construct
  REGION 1..12: note = Synthetic
DCVIGIRDDSDI

SEQ ID NO: 131 - protein, 14 aa, synthetic construct
  REGION 1..14: note = Synthetic
GITIHGVVIIPPDY

SEQ ID NO: 132 - protein, 13 aa, synthetic construct
  REGION 1..13: note = Synthetic
EYYGSENYDHFDY

SEQ ID NO: 133 - protein, 9 aa, synthetic construct
  REGION 1..9: note = Synthetic
GAFRTTMDY

SEQ ID NO: 134 - protein, 11 aa, synthetic construct
  REGION 1..11: note = Synthetic
WGQGTLVTVSS

SEQ ID NO: 135 - protein, 11 aa, synthetic construct
  REGION 1..11: note = Synthetic
WGQGALVTVSS

SEQ ID NO: 136 - protein, 11 aa, synthetic construct
  REGION 1..11: note = Synthetic
WGQGTPVTVSS

SEQ ID NO: 137 - protein, 11 aa, synthetic construct
  REGION 1..11: note = Synthetic
WGQGTMVTVSS

SEQ ID NO: 138 - protein, 11 aa, synthetic construct
  REGION 1..11: note = Synthetic
RGQGTLVTVSS

SEQ ID NO: 139 - protein, 23 aa, synthetic construct
  REGION 1..23: note = Synthetic
DIQMTQSPSSLSASVGDRVTINC

SEQ ID NO: 140 - protein, 11 aa, synthetic construct
  REGION 1..11: note = Synthetic
QASQSIDSNLA

SEQ ID NO: 141 - protein, 15 aa, synthetic construct
  REGION 1..15: note = Synthetic
WFQQKPGQPPKLLIY

SEQ ID NO: 142 - protein, 7 aa, synthetic construct
  REGION 1..7: note = Synthetic
RASNLAS

SEQ ID NO: 143 - protein, 32 aa, synthetic construct
  REGION 1..32: note = Synthetic
GVPDRFSGSGSGTDFTLTISSLEAEDVATYYC

SEQ ID NO: 144 - protein, 12 aa, synthetic construct
  REGION 1..12: note = Synthetic
LGGVGAVSYRTS

SEQ ID NO: 145 - protein, 10 aa, synthetic construct
  REGION 1..10: note = Synthetic
FGGGTKVEIK

SEQ ID NO: 146 - protein, 119 aa, synthetic construct
  REGION 1..119: note = Synthetic
EVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSS

SEQ ID NO: 147 - protein, 125 aa, synthetic construct
  REGION 1..125: note = Synthetic
EVQLVESGGGLVKPGGSLRLSCAASGFIFSDYYMSWIRQAPGKGLEWVSYISSSGSTIHYADSVKGRFTVSRDNAKNSLYLQMNSLRTEDTAVYYCARDPPTSNSDWVSLHFDHWGQGTLVTVSS

SEQ ID NO: 148 - protein, 125 aa, synthetic construct
  REGION 1..125: note = Synthetic
EVQLVESGGGLVKPGGSLRLSCAASGFTFSDYYMSWIRQAPGKGLEWISYISSSGTTIHYADSVKGRFTISRDNAKNSLYLQMNSLRTEDTAVYYCARDAPSSNSDWVSLQFDYWGQGTLVTVSS

SEQ ID NO: 149 - protein, 121 aa, synthetic construct
  REGION 1..121: note = Synthetic
EVQLVESGGGLVKPGGSLKLSCAASGFTFSDYYMSWIRQAPGKGVEWISYISNNGSTIHYADSVKGRFTISRDNAKNSLYLQMNSLRAEDTAVYYCARDFNNGWYEDFDYWGQGTLVTVSS

SEQ ID NO: 150 - protein, 125 aa, synthetic construct
  REGION 1..125: note = Synthetic
EVQLVESGGGLVKPGGSLRLSCAASGFTFSDYYMSWIRQAPGKGLEWISYISSSGSTIHYADSVKGRFTISRDNAKNSLYLQMNSLRAEDTAIYYCARDIPSSSSDWVSLQFDYWGQGTLVTVSS

SEQ ID NO: 151 - protein, 123 aa, synthetic construct
  REGION 1..123: note = Synthetic
EVQLVESGGGLVKPGGSLRLSCVASGFTFSDYYMSWIRQAPGKGLEWISYISSSGSSIYYAESVKGRFTISRDNAKNSLYLQMNRLRAEDTALYYCARTPPSSDNWYEDFDYWGQGALVTVSS

SEQ ID NO: 152 - protein, 121 aa, synthetic construct
  REGION 1..121: note = Synthetic
EVQLVESGGGLVKPGGSLRLSCAASGFNFSDYYMSWIRQAPGKGLEWISYISNSSSTIYYADSVKGRFTISRDNAKNSLYLQMSRLRAEDTAMYYCARDTTNGWYEDFDYWGQGTLVTVSS

SEQ ID NO: 153 - protein, 123 aa, synthetic construct
  REGION 1..123: note = Synthetic
EVQLVESGGGLVKPGGSLRLSCVASGFTFSDYYMSWIRQAPGKGMEWISYISSSGSTKNYANSVKGRFTISRDNAKNSLYLQMNSLRAEDTAAYYCARVPPYNASWYEDFDYWGQGTLVTVSS

SEQ ID NO: 154 - protein, 121 aa, synthetic construct
  REGION 1..121: note = Synthetic
EVQLVESGGGLVKPGGSLRLSCAASGFTFSDYYMSWIRQAPGKGLEWISYISNSSSSISYANSVKGRFTVSRDNAKNSLYLQMNSLRAEDTALYYCARSPRGAFYEDFDYWGQGTLVTVSS

SEQ ID NO: 155 - protein, 125 aa, synthetic construct
  REGION 1..125: note = Synthetic
EVQLVESGGGLVKPGGSLRLSCAASGFTFSDYYMSWIRQAPGKGLEWIAYISSSGSTIHYSDSVKGRFTISRDNAKNSLYLQMNSLRAEDTAVYYCARDTPSSSSDWVSLQFDYWGQGTPVTVSS

SEQ ID NO: 156 - protein, 120 aa, synthetic construct
  REGION 1..120: note = Synthetic
EVQLVESGGGLVKPGGSLRLSCAASGFTLSDYYMSWIRQAPGKGLEWVSNISKNGFTIYYAESVKGRFTVSRDNAKNSLYLQMNSLRAEDTAIYYCARDSSGWYSEFDYWGQGTLVTVSS

SEQ ID NO: 157 - protein, 123 aa, synthetic construct
  REGION 1..123: note = Synthetic
EVQLVESGGGLVKPGGSLRLSCVTSGFTFSDYYMSWIRQAPGKGMEWISYISTTGSTKNYANSVKGRFTISRDNAKSSLYLQMNSLRAEDTAAYYCARVPPSNASWYEDFDYWGQGTLVTVSS

SEQ ID NO: 158 - protein, 125 aa, synthetic construct
  REGION 1..125: note = Synthetic
EVQLVESGGGLVKPGGSLRLSCAASGFTFSDYYMSWIRQTPGKGLEWVSYISRSGSTKYYADSVKGRFTISRDNAKNSLYLQMNSLRAEDTAVYYCARDAPSSNSDWVSLHFDHWGQGTLVTVSS

SEQ ID NO: 159 - protein, 121 aa, synthetic construct
  REGION 1..121: note = Synthetic
EVQLVESGGGLVKPGGSLRLSCAASGFTFSDYYMSWIRQAPGKGLEWISYISNSSSSISYANSVKGRFTVSRDNAKNSLYLQMNSLRAEDTALYYCARSPRSAFYEDFDYWGQGTLVTVSS

SEQ ID NO: 160 - protein, 123 aa, synthetic construct
  REGION 1..123: note = Synthetic
EVQLVESGGGLVKPGGSLRLSCAASGFTLSDSQMSWIRQAPGKGVEWVSYISSSGNTIYYGDSVKGRFTISRDNAKNSLFLQMNSLRAEDTAVYYCARVPPSSSNWYEDFDIWGQGTLVTVSS

SEQ ID NO: 161 - protein, 123 aa, synthetic construct
  REGION 1..123: note = Synthetic
QVQLVESGGGLVKPGGSLRLSCAASGFKLSDFQMSWIRQAPGKGLEWVAYIDTNGSTRYYAESVKGRFTLSRDNVKNSLNLQMNGLRAEDTALYYCARIPSYTSSWYEDFDHWGQGTLVTVSS

SEQ ID NO: 162 - protein, 123 aa, synthetic construct
  REGION 1..123: note = Synthetic
EVQLVESGGGLVKPGGSLRLSCAASGFTFSDYYMSWIRQAPGKGLEWISYISSSGGSIYYADSVKGRFTISRDNAKNSLYLQMNRLRAEDTALYYCARTPPSSNNWYEDFDYWGQGALVTVSS

SEQ ID NO: 163 - protein, 125 aa, synthetic construct
  REGION 1..125: note = Synthetic
EVQLVESGGGLVKPGGSLRLSCAASGFTFSDFYMSWIRQAPGKGLEWISYISSSGSYIHYADSVKGRFTISRDNAKNSLYLQMNSLRAEDTAIYYCARDMPSSSSDWVDLQFDYWGQGTLVTVSS

SEQ ID NO: 164 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYADSVKGRFTISRDNAKNSLYLQMNSLRAEDTAIYYCARDIPSSSSDWVDLQFDYWGQGTLVTVSS

SEQ ID NO: 165 - protein, 125 aa, synthetic construct
  REGION 1..125: note = Synthetic
EVQLVESGGGLVKPGGSLRLSCAASGFTFSDFYMSWIRQAPGKGLEWISYISSSGSYIHYADSVKGRFTISRDNAKNSLYLQMNSLRAEDTAIYYCARDIPSSSSDWVDLQFDYWGQGTLVTVSS

SEQ ID NO: 166 - protein, 125 aa, synthetic construct
  REGION 1..125: note = Synthetic
EVQLVESGGGLVKPGGSLRLSCAASGFTFSDFYMSWIRQAPGKGLEWISYISSSGSTIHYADSVKGRFTISRDNAKNSLYLQMNSLRAEDTAIYYCARDMPSSSSDWVDLQFDYWGQGTLVTVSS

SEQ ID NO: 167 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYADSVKGRFTISRDNAKNSLYLQMNSLRAEDTAIYYCARDMPSSSSDWVSLQFDYWGQGTLVTVSS

SEQ ID NO: 168 - protein, 125 aa, synthetic construct
  REGION 1..125: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDMPSSSSDWVDLQFDYWGQGTLVTVSS

SEQ ID NO: 169 - protein, 125 aa, synthetic construct
  REGION 1..125: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDMPSSSSDWVDLQFDYWGQGTLVTVSS

SEQ ID NO: 170 - protein, 125 aa, synthetic construct
  REGION 1..125: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDMPSSSSDWVDLQFDYWGQGTLVTVSS

SEQ ID NO: 171 - protein, 125 aa, synthetic construct
  REGION 1..125: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDIPSSSSDWVDLQFDYWGQGTLVTVSS

SEQ ID NO: 172 - protein, 125 aa, synthetic construct
  REGION 1..125: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDIPSSSSDWVDLQFDYWGQGTLVTVSS

SEQ ID NO: 173 - protein, 125 aa, synthetic construct
  REGION 1..125: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDIPSSSSDWVDLQFDYWGQGTLVTVSS

SEQ ID NO: 174 - protein, 125 aa, synthetic construct
  REGION 1..125: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDMPSSSSDWVSLQFDYWGQGTLVTVSS

SEQ ID NO: 175 - protein, 125 aa, synthetic construct
  REGION 1..125: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDMPSSSSDWVSLQFDYWGQGTLVTVSS

SEQ ID NO: 176 - protein, 125 aa, synthetic construct
  REGION 1..125: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDMPSSSSDWVSLQFDYWGQGTLVTVSS

SEQ ID NO: 177 - protein, 125 aa, synthetic construct
  REGION 1..125: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDIPSSSSDWVSLQFDYWGQGTLVTVSS

SEQ ID NO: 178 - protein, 125 aa, synthetic construct
  REGION 1..125: note = Synthetic
EVQLVESGGGLVKPGGSLRLSCAASGFTFSDYYMSWIRQAPGKGLEWISYIDSSGRPLAYADSVKGRFTISRDNAKNSLYLQMNSLRAEDTAIYYCARDIPSSSSDWVDLQFDYWGQGTLVTVSS

SEQ ID NO: 179 - protein, 125 aa, synthetic construct
  REGION 1..125: note = Synthetic
EVQLVESGGGLVKPGGSLRLSCAASGFTFSDYYMSWIRQAPGKGLEWISYIDSSGRPLAYADSVKGRFTISRDNAKNSLYLQMNSLRAEDTAIYYCARDMPSSSSDWVSLQFDYWGQGTLVTVSS

SEQ ID NO: 180 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYAHSVKGRFTISRDNAKNSLYLQMNSLRAEDTAIYYCARDMPSSSEDWVDLQFDYWGQGTLVTVSS

SEQ ID NO: 181 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSGSVKGRFTISRDNAKNSLYLQMNSLRAEDTAIYYCARDMPSSSSDWVDLQFDYWGQGTLVTVSS

SEQ ID NO: 182 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSHSVKGRFTISRDNAKNSLYLQMNSLRAEDTAIYYCARDMPSSSEDWVDLQFDYWGQGTLVTVSS

SEQ ID NO: 183 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSIDWVELQFDYWGQGTLVTVSS

SEQ ID NO: 184 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSPSVKGRFTISRDNAKNSLYLQMNSLRAEDTAIYYCARDMPSSSKDWVDLQFDYWGQGTLVTVSS

SEQ ID NO: 185 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSPSVKGRFTISRDNAKNSLYLQMNSLRAEDTAIYYCARDMPSSSIDWVDLQFDYWGQGTLVTVSS

SEQ ID NO: 186 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSASVKGRFTISRDNAKNSLYLQMNSLRAEDTAIYYCARDMPSSSIDWVELQFDYWGQGTLVTVSS

SEQ ID NO: 187 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSTSVKGRFTISRDNAKNSLYLQMNSLRAEDTAIYYCARDMPSSSSDWVDLQFDYWGQGTLVTVSS

SEQ ID NO: 188 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSEDWVELQFDYWGQGTLVTVSS

SEQ ID NO: 189 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSSDWVDLQFDYWGQGTLVTVSS

SEQ ID NO: 190 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYAGSVKGRFTISRDNAKNSLYLQMNSLRAEDTAIYYCARDMPSSSSDWVELQFDYWGQGTLVTVSS

SEQ ID NO: 191 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSEDWVMLQFDYWGQGTLVTVSS

SEQ ID NO: 192 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYATSVKGRFTISRDNAKNSLYLQMNSLRAEDTAIYYCARDMPSSSIDWVDLQFDYWGQGTLVTVSS

SEQ ID NO: 193 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYASSVKGRFTISRDNAKNSLYLQMNSLRAEDTAIYYCARDMPSSSSDWVDLQFDYWGQGTLVTVSS

SEQ ID NO: 194 - protein, 125 aa, synthetic construct
  REGION 1..125: note = Synthetic
EVQLVESGGGLVKPGGSLRLSCAASGFTFSDFYMSWIRQAPGKGLEWISYISSSGSWIHYSTSVKGRFTISRDNAKNSLYLQMNSLRAEDTAIYYCARDMPSSSIDWVDLQFDYWGQGTLVTVSS

SEQ ID NO: 195 - protein, 124 aa, synthetic construct
  REGION 1..124: note = Synthetic
EVQLLESGGGLVQPGGSLRLSCAASGFTFSSHAMSWVRQAPGKGLEWVSAISGSGDSTHYAASVKGRFTISRDNSKNTLYLQMNSLRAEDTAVYYCETLLRFLESLGNDGFKIWGQGTMVTVSS

SEQ ID NO: 196 - protein, 121 aa, synthetic construct
  REGION 1..121: note = Synthetic
QVQLVESGGGLVKPGGSLRISCAASGFTFSNYNMSWIRQAPGKGVEWVSHISGSGRTIYYAESVKGRFTISRDNAKNSLYLQMNSLRAEDTAMYYCARDLSSGWYEDFDYWGQGTLVTVSS

SEQ ID NO: 197 - protein, 120 aa, synthetic construct
  REGION 1..120: note = Synthetic
EVQLVESGGGLVQPGGSLRLSCAASGFTFSNFWMYWVRQAPGKGLVWVSRINSDGSSTNYADSVKGRFTISRDNAKNTLYLQMNSLRVEDTAVYSCAREGSGWYEDFDYWGQGTLVTVSS

SEQ ID NO: 198 - protein, 121 aa, synthetic construct
  REGION 1..121: note = Synthetic
EVQLVESGGGLVKPGGSLRLSCAASGFTLSDYYMSWIRQAPGKGLEWISYISSNGSTIYYADSVKGRFTISRDNARNSLYLQMNSLRAEDTALYYCARDVSSGWYEDFDYWGQGTLVTVSS

SEQ ID NO: 199 - protein, 121 aa, synthetic construct
  REGION 1..121: note = Synthetic
EVQLVESGGGLVKPGGSLRLSCAASGFNLSDSYMSWIRQAPGKGLEWVSCISSSGSTIYYADSVKGRFTVSRDNAKNSLYLQMNHLRAEDTALYYCARDCVIGIRDDSDIWGQGTMVTVSS

SEQ ID NO: 200 - protein, 123 aa, synthetic construct
  REGION 1..123: note = Synthetic
EVQLLESGGGLVQPGGSLRLSCAASGFIFGSYAMSWVRQAPGKGLEWVSGISGTGGNTYYADSVKGRFTISRDNSKNTLYLQMNSLRAEDTAVYFCERGITIHGVVIIPPDYRGQGTLVTVSS

SEQ ID NO: 201 - protein, 122 aa, synthetic construct
  REGION 1..122: note = Synthetic
EVQLVESGGGLVKPGGSLRLSCAASGFTFSDYYMSWIRQAPGKGLEWIAYISSSGSTIYYADSVKGRFTISRDNAKNSLYLQMNSLRAEDTAIYYCAREYYGSENYDHFDYWGQGTLVTVSS

SEQ ID NO: 202 - protein, 118 aa, synthetic construct
  REGION 1..118: note = Synthetic
EVQLLESGGGLVQPGGSLRLSCAASGFTFSGNAMSWVRQAPGKGLEWVSAISGSGDKTYYAASVKGRFTISRDNSNNTVYLQMNSLRAEDTAVYYCEKGAFRTTMDYWGQGTLVTVSS

SEQ ID NO: 203 - protein, 110 aa, synthetic construct
  REGION 1..110: note = Synthetic
DIQMTQSPSSLSASVGDRVTINCQASQSIDSNLAWFQQKPGQPPKLLIYRASNLASGVPDRFSGSGSGTDFTLTISSLEAEDVATYYCLGGVGAVSYRTSFGGGTKVEIK

SEQ ID NO: 204 - protein, 449 aa, synthetic construct
  REGION 1..449: note = Synthetic
EVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 357 aa, synthetic construct
  REGION 1..357: note = Synthetic
EVQLVESGGGLVKPGGSLRLSCAASGFIFSDYYMSWIRQAPGKGLEWVSYISSSGSTIHYADSVKGRFTVSRDNAKNSLYLQMNSLRTEDTAVYYCARDPPTSNSDWVSLHFDHWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 357 aa, synthetic construct
  REGION 1..357: note = Synthetic
EVQLVESGGGLVKPGGSLRLSCAASGFTFSDYYMSWIRQAPGKGLEWISYISSSGTTIHYADSVKGRFTISRDNAKNSLYLQMNSLRTEDTAVYYCARDAPSSNSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 353 aa, synthetic construct
  REGION 1..353: note = Synthetic
EVQLVESGGGLVKPGGSLKLSCAASGFTFSDYYMSWIRQAPGKGVEWISYISNNGSTIHYADSVKGRFTISRDNAKNSLYLQMNSLRAEDTAVYYCARDFNNGWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 357 aa, synthetic construct
  REGION 1..357: note = Synthetic
EVQLVESGGGLVKPGGSLRLSCAASGFTFSDYYMSWIRQAPGKGLEWISYISSSGSTIHYADSVKGRFTISRDNAKNSLYLQMNSLRAEDTAIYYCARDIPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 355 aa, synthetic construct
  REGION 1..355: note = Synthetic
EVQLVESGGGLVKPGGSLRLSCVASGFTFSDYYMSWIRQAPGKGLEWISYISSSGSSIYYAESVKGRFTISRDNAKNSLYLQMNRLRAEDTALYYCARTPPSSDNWYEDFDYWGQGALVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 353 aa, synthetic construct
  REGION 1..353: note = Synthetic
EVQLVESGGGLVKPGGSLRLSCAASGFNFSDYYMSWIRQAPGKGLEWISYISNSSSTIYYADSVKGRFTISRDNAKNSLYLQMSRLRAEDTAMYYCARDTTNGWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 355 aa, synthetic construct
  REGION 1..355: note = Synthetic
EVQLVESGGGLVKPGGSLRLSCVASGFTFSDYYMSWIRQAPGKGMEWISYISSSGSTKNYANSVKGRFTISRDNAKNSLYLQMNSLRAEDTAAYYCARVPPYNASWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 353 aa, synthetic construct
  REGION 1..353: note = Synthetic
EVQLVESGGGLVKPGGSLRLSCAASGFTFSDYYMSWIRQAPGKGLEWISYISNSSSSISYANSVKGRFTVSRDNAKNSLYLQMNSLRAEDTALYYCARSPRGAFYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 357 aa, synthetic construct
  REGION 1..357: note = Synthetic
EVQLVESGGGLVKPGGSLRLSCAASGFTFSDYYMSWIRQAPGKGLEWIAYISSSGSTIHYSDSVKGRFTISRDNAKNSLYLQMNSLRAEDTAVYYCARDTPSSSSDWVSLQFDYWGQGTPVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 352 aa, synthetic construct
  REGION 1..352: note = Synthetic
EVQLVESGGGLVKPGGSLRLSCAASGFTLSDYYMSWIRQAPGKGLEWVSNISKNGFTIYYAESVKGRFTVSRDNAKNSLYLQMNSLRAEDTAIYYCARDSSGWYSEFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 355 aa, synthetic construct
  REGION 1..355: note = Synthetic
EVQLVESGGGLVKPGGSLRLSCVTSGFTFSDYYMSWIRQAPGKGMEWISYISTTGSTKNYANSVKGRFTISRDNAKSSLYLQMNSLRAEDTAAYYCARVPPSNASWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 357 aa, synthetic construct
  REGION 1..357: note = Synthetic
EVQLVESGGGLVKPGGSLRLSCAASGFTFSDYYMSWIRQTPGKGLEWVSYISRSGSTKYYADSVKGRFTISRDNAKNSLYLQMNSLRAEDTAVYYCARDAPSSNSDWVSLHFDHWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 353 aa, synthetic construct
  REGION 1..353: note = Synthetic
EVQLVESGGGLVKPGGSLRLSCAASGFTFSDYYMSWIRQAPGKGLEWISYISNSSSSISYANSVKGRFTVSRDNAKNSLYLQMNSLRAEDTALYYCARSPRSAFYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 355 aa, synthetic construct
  REGION 1..355: note = Synthetic
EVQLVESGGGLVKPGGSLRLSCAASGFTLSDSQMSWIRQAPGKGVEWVSYISSSGNTIYYGDSVKGRFTISRDNAKNSLFLQMNSLRAEDTAVYYCARVPPSSSNWYEDFDIWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 355 aa, synthetic construct
  REGION 1..355: note = Synthetic
QVQLVESGGGLVKPGGSLRLSCAASGFKLSDFQMSWIRQAPGKGLEWVAYIDTNGSTRYYAESVKGRFTLSRDNVKNSLNLQMNGLRAEDTALYYCARIPSYTSSWYEDFDHWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 355 aa, synthetic construct
  REGION 1..355: note = Synthetic
EVQLVESGGGLVKPGGSLRLSCAASGFTFSDYYMSWIRQAPGKGLEWISYISSSGGSIYYADSVKGRFTISRDNAKNSLYLQMNRLRAEDTALYYCARTPPSSNNWYEDFDYWGQGALVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 357 aa, synthetic construct
  REGION 1..357: note = Synthetic
EVQLVESGGGLVKPGGSLRLSCAASGFTFSDFYMSWIRQAPGKGLEWISYISSSGSYIHYADSVKGRFTISRDNAKNSLYLQMNSLRAEDTAIYYCARDMPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 357 aa, synthetic construct
  REGION 1..357: note = Synthetic
EVQLVESGGGLVKPGGSLRLSCAASGFTFSDFYMSWIRQAPGKGLEWISYISSSGSWIHYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 357 aa, synthetic construct
  REGION 1..357: note = Synthetic
EVQLVESGGGLVKPGGSLRLSCAASGFTFSDFYMSWIRQAPGKGLEWISYISSSGSYIHYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 357 aa, synthetic construct
  REGION 1..357: note = Synthetic
EVQLVESGGGLVKPGGSLRLSCAASGFTFSDFYMSWIRQAPGKGLEWISYISSSGSTIHYADSVKGRFTISRDNAKNSLYLQMNSLRAEDTAIYYCARDMPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 357 aa, synthetic construct
  REGION 1..357: note = Synthetic
EVQLVESGGGLVKPGGSLRLSCAASGFTFSDFYMSWIRQAPGKGLEWISYISSSGSWIHYADSVKGRFTISRDNAKNSLYLQMNSLRAEDTAIYYCARDMPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 357 aa, synthetic construct
  REGION 1..357: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDMPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 357 aa, synthetic construct
  REGION 1..357: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDMPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 357 aa, synthetic construct
  REGION 1..357: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDMPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 357 aa, synthetic construct
  REGION 1..357: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 357 aa, synthetic construct
  REGION 1..357: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 357 aa, synthetic construct
  REGION 1..357: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 357 aa, synthetic construct
  REGION 1..357: note = Synthetic
EVQLVESGGGLVKPGGSLRLSCAASGFTFSDFYMSWIRQAPGKGLEWISYINTRGSPIGYADSVKGRFTISRDNAKNSLYLQMNSLRAEDTAIYYCARDMPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 357 aa, synthetic construct
  REGION 1..357: note = Synthetic
EVQLVESGGGLVKPGGSLRLSCAASGFTFSDFYMSWIRQAPGKGLEWISYISSSGSTRYYADSVKGRFTISRDNAKNSLYLQMNSLRAEDTAIYYCARDMPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 357 aa, synthetic construct
  REGION 1..357: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDMPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 357 aa, synthetic construct
  REGION 1..357: note = Synthetic
EVQLVESGGGLVKPGGSLRLSCAASGFTFSDFYMSWIRQAPGKGLEWISYIDSSGRPLAYADSVKGRFTISRDNAKNSLYLQMNSLRAEDTAIYYCARDIPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 357 aa, synthetic construct
  REGION 1..357: note = Synthetic
EVQLVESGGGLVKPGGSLRLSCAASGFTFSDYYMSWIRQAPGKGLEWISYIDSSGRPLAYADSVKGRFTISRDNAKNSLYLQMNSLRAEDTAIYYCARDIPSSSSDWVDLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 357 aa, synthetic construct
  REGION 1..357: note = Synthetic
EVQLVESGGGLVKPGGSLRLSCAASGFTFSDYYMSWIRQAPGKGLEWISYIDSSGRPLAYADSVKGRFTISRDNAKNSLYLQMNSLRAEDTAIYYCARDMPSSSSDWVSLQFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYAHSVKGRFTISRDNAKNSLYLQMNSLRAEDTAIYYCARDMPSSSEDWVDLQFDYWGQGTLVTVSSQGGSDYKDDDDKASHHHHHH

SEQ ID NO: 239 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSGSVKGRFTISRDNAKNSLYLQMNSLRAEDTAIYYCARDMPSSSSDWVDLQFDYWGQGTLVTVSSQGGSDYKDDDDKASHHHHHH

SEQ ID NO: 240 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSHSVKGRFTISRDNAKNSLYLQMNSLRAEDTAIYYCARDMPSSSEDWVDLQFDYWGQGTLVTVSSQGGSDYKDDDDKASHHHHHH

SEQ ID NO: 241 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSIDWVELQFDYWGQGTLVTVSSQGGSDYKDDDDKASHHHHHH

SEQ ID NO: 242 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSPSVKGRFTISRDNAKNSLYLQMNSLRAEDTAIYYCARDMPSSSKDWVDLQFDYWGQGTLVTVSSQGGSDYKDDDDKASHHHHHH

SEQ ID NO: 243 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSPSVKGRFTISRDNAKNSLYLQMNSLRAEDTAIYYCARDMPSSSIDWVDLQFDYWGQGTLVTVSSQGGSDYKDDDDKASHHHHHH

SEQ ID NO: 244 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSASVKGRFTISRDNAKNSLYLQMNSLRAEDTAIYYCARDMPSSSIDWVELQFDYWGQGTLVTVSSQGGSDYKDDDDKASHHHHHH

SEQ ID NO: 245 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSTSVKGRFTISRDNAKNSLYLQMNSLRAEDTAIYYCARDMPSSSSDWVDLQFDYWGQGTLVTVSSQGGSDYKDDDDKASHHHHHH

SEQ ID NO: 246 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSEDWVELQFDYWGQGTLVTVSSQGGSDYKDDDDKASHHHHHH

SEQ ID NO: 247 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSSDWVDLQFDYWGQGTLVTVSSQGGSDYKDDDDKASHHHHHH

SEQ ID NO: 248 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYAGSVKGRFTISRDNAKNSLYLQMNSLRAEDTAIYYCARDMPSSSSDWVELQFDYWGQGTLVTVSSQGGSDYKDDDDKASHHHHHH

SEQ ID NO: 249 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSSSVKGRFTISRDNAKNSLYLQMNSLRAEDTAIYYCARDMPSSSEDWVMLQFDYWGQGTLVTVSSQGGSDYKDDDDKASHHHHHH

SEQ ID NO: 250 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYATSVKGRFTISRDNAKNSLYLQMNSLRAEDTAIYYCARDMPSSSIDWVDLQFDYWGQGTLVTVSSQGGSDYKDDDDKASHHHHHH

SEQ ID NO: 251 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYASSVKGRFTISRDNAKNSLYLQMNSLRAEDTAIYYCARDMPSSSSDWVDLQFDYWGQGTLVTVSSQGGSDYKDDDDKASHHHHHH

SEQ ID NO: 252 - protein, 145 aa, synthetic construct
  REGION 1..145: note = Synthetic
EVQLVESGGGLVKPGGSLRLSCAASGFTFSDFYMSWIRQAPGKGLEWISYISSSGSWIHYSTSVKGRFTISRDNAKNSLYLQMNSLRAEDTAIYYCARDMPSSSIDWVDLQFDYWGQGTLVTVSSQGGSDYKDDDDKASHHHHHH

SEQ ID NO: 253 - protein, 356 aa, synthetic construct
  REGION 1..356: note = Synthetic
EVQLLESGGGLVQPGGSLRLSCAASGFTFSSHAMSWVRQAPGKGLEWVSAISGSGDSTHYAASVKGRFTISRDNSKNTLYLQMNSLRAEDTAVYYCETLLRFLESLGNDGFKIWGQGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 353 aa, synthetic construct
  REGION 1..353: note = Synthetic
QVQLVESGGGLVKPGGSLRISCAASGFTFSNYNMSWIRQAPGKGVEWVSHISGSGRTIYYAESVKGRFTISRDNAKNSLYLQMNSLRAEDTAMYYCARDLSSGWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 352 aa, synthetic construct
  REGION 1..352: note = Synthetic
EVQLVESGGGLVQPGGSLRLSCAASGFTFSNFWMYWVRQAPGKGLVWVSRINSDGSSTNYADSVKGRFTISRDNAKNTLYLQMNSLRVEDTAVYSCAREGSGWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 353 aa, synthetic construct
  REGION 1..353: note = Synthetic
EVQLVESGGGLVKPGGSLRLSCAASGFTLSDYYMSWIRQAPGKGLEWISYISSNGSTIYYADSVKGRFTISRDNARNSLYLQMNSLRAEDTALYYCARDVSSGWYEDFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 353 aa, synthetic construct
  REGION 1..353: note = Synthetic
EVQLVESGGGLVKPGGSLRLSCAASGFNLSDSYMSWIRQAPGKGLEWVSCISSSGSTIYYADSVKGRFTVSRDNAKNSLYLQMNHLRAEDTALYYCARDCVIGIRDDSDIWGQGTMVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 355 aa, synthetic construct
  REGION 1..355: note = Synthetic
EVQLLESGGGLVQPGGSLRLSCAASGFIFGSYAMSWVRQAPGKGLEWVSGISGTGGNTYYADSVKGRFTISRDNSKNTLYLQMNSLRAEDTAVYFCERGITIHGVVIIPPDYRGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 354 aa, synthetic construct
  REGION 1..354: note = Synthetic
EVQLVESGGGLVKPGGSLRLSCAASGFTFSDYYMSWIRQAPGKGLEWIAYISSSGSTIYYADSVKGRFTISRDNAKNSLYLQMNSLRAEDTAIYYCAREYYGSENYDHFDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 350 aa, synthetic construct
  REGION 1..350: note = Synthetic
EVQLLESGGGLVQPGGSLRLSCAASGFTFSGNAMSWVRQAPGKGLEWVSAISGSGDKTYYAASVKGRFTISRDNSNNTVYLQMNSLRAEDTAVYYCEKGAFRTTMDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 217 aa, synthetic construct
  REGION 1..217: note = Synthetic
DIQMTQSPSSLSASVGDRVTINCQASQSIDSNLAWFQQKPGQPPKLLIYRASNLASGVPDRFSGSGSGTDFTLTISSLEAEDVATYYCLGGVGAVSYRTSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - DNA, 24 bp, synthetic construct
  misc_feature 1..24: note = Primer
  source 1..24: mol_type = other DNA
ggtgtccagtgtsaggtgcagctg

SEQ ID NO: 263 - DNA, 29 bp, synthetic construct
  misc_feature 1..29: note = Primer
  source 1..29: mol_type = other DNA
aatccctgggcactgaagagacggtgacc

(19) United States
(12) Patent Application Publication
(10) Pub. No.: US 2025/0257130 A1
(43) Pub. Date: Aug. 14, 2025

(54) ANTI-ROR1 ANTIBODIES

(71) Applicant: NONA BIOSCIENCES (SUZHOU) CO., LTD., Suzhou (CN)

(72) Inventors: Jianxun Zhao, Suzhou (CN); Cui Nie, Suzhou (CN); Zheng Wang, Suzhou (CN); Yue Gao, Suzhou (CN); Chuchu Zhao, Suzhou (CN); Guangbei Zhu, Suzhou (CN); Jianhao Hou, Suzhou (CN); Yuanyuan Yang, Suzhou (CN)

(21) Appl. No.: 18/880,384

(22) PCT Filed: Jul. 5, 2023
(86) PCT No.: PCT/CN2023/105835

§ 371 Date: Dec. 31, 2024

(30) Foreign Application Priority Data
Jul. 6, 2022 (WO) PCT/CN2022/104152

(51) Int. Cl. C07K 16/28; A61K 45/06; A61K 47/68

(52) U.S. Cl. CPC C07K 16/2803; A61K 45/06; A61K 47/6879; C07K 16/2809; C07K 2317/31; C07K 2317/565; C07K 2317/569; C07K 2317/76; C07K 2317/77; C07K 2317/92

(57) ABSTRACT

Provided are antibodies and antigen-binding fragments thereof that recognize ROR1. In some embodiments, the antibodies provide a means of treating ROR1-positive cancer. In some embodiments, the antibodies are used to diagnose or image ROR1-positive cancer.

### FIELD OF THE INVENTION

The invention relates to antibodies and antigen-binding fragments thereof that bind to ROR1 and use thereof.

### BACKGROUND OF THE INVENTION

Receptor tyrosine kinase like orphan receptor 1 (ROR1) is highly expressed during embryonic and infant development, and the expression level decreases significantly in children and adults. The expression of ROR1 was significantly increased in a variety of blood cancers and solid tumors. Blood cancers that highly express ROR1 include, e.g., B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL) and marginal zone lymphoma (MZL). In solid tumors, the types of cancers expressing ROR1 include, e.g., breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, adrenal cancer and many other cancers. Therefore, ROR1 has become a new tumor specific target. Many data show that ROR1 plays an important role in promoting tumor growth and metastasis, inducing drug resistance, and inhibiting apoptosis.

The current consensus about ROR1 signal transduction is that ROR1 can play an important role in a variety of physiological processes, including regulating cell division, proliferation, migration, and cell chemotaxis, especially wnt5a, by mediating the signal transmission of non-classical wnt pathways. Wnt5a is a typical non-classical activator of wnt signaling pathway and participates in phosphorylation of NF-κB subunit p65, activating NF-κB pathway in tumor cells, promoting cell migration and invasion, EMT, cancer metastasis, etc. Wnt5a/ROR1 is highly expressed in many cancers. As the receptor of Wnt5a, ROR1 participates in the activation of tumor cell NF-κB channel.

ROR1 is considered a potential target because it is a tyrosine kinase receptor with drug resistance; and it is expressed on the cell surface. More importantly, it is highly expressed in tumor cells, but very low in healthy adult tissues.

There is a lack of antibodies that have high affinity with ROR1, do not cross react with ROR2 and have good binding ability and internalization activity, especially fully human antibodies.

### SUMMARY OF THE INVENTION

The present invention provides novel antibodies binding to ROR1 or antigen binding fragments thereof, which can be in a form of a heavy chain-only antibody (HCAb).

In an aspect, the invention provides an antibody that specifically binds to ROR1, or an antigen binding fragment thereof, wherein the antibody comprises a heavy chain variable region (VH), and wherein the VH comprises HCDRs 1-3 of a VH having the amino acid sequence set forth in any one of SEQ ID NOs: 147-202.

In some embodiments, the VH comprises HCDRs 1-3 having the amino acid sequences set forth in:
SEQ ID NOs: 11, 40, 100 respectively,
SEQ ID NOs: 12, 41, 101 respectively,
SEQ ID NOs: 12, 42, 102 respectively,
SEQ ID NOs: 12, 40, 103 respectively,
SEQ ID NOs: 12, 43, 104 respectively,
SEQ ID NOs: 13, 44, 105 respectively,
SEQ ID NOs: 12, 40, 106 respectively,
SEQ ID NOs: 12, 45, 107 respectively,
SEQ ID NOs: 12, 40, 108 respectively,
SEQ ID NOs: 14, 46, 109 respectively,
SEQ ID NOs: 12, 47, 110 respectively,
SEQ ID NOs: 12, 48, 111 respectively,
SEQ ID NOs: 12, 45, 112 respectively,
SEQ ID NOs: 15, 49, 113 respectively,
SEQ ID NOs: 16, 50, 114 respectively,
SEQ ID NOs: 12, 51, 115 respectively,
SEQ ID NOs: 18, 56, 126 respectively,
SEQ ID NOs: 21, 40, 130 respectively,
SEQ ID NOs: 22, 60, 131 respectively,
SEQ ID NOs: 12, 40, 132 respectively,
SEQ ID NOs: 23, 61, 133 respectively,
SEQ ID NOs: 19, 57, 127 respectively,
SEQ ID NOs: 20, 58, 128 respectively,
SEQ ID NOs: 14, 59, 129 respectively,
SEQ ID NOs: 17, 53, 119 respectively,
SEQ ID NOs: 17, 53, 116 respectively,
SEQ ID NOs: 17, 53, 120 respectively,
SEQ ID NOs: 17, 53, 121 respectively,
SEQ ID NOs: 17, 53, 122 respectively,
SEQ ID NOs: 17, 53, 123 respectively,
SEQ ID NOs: 17, 53, 124 respectively,
SEQ ID NOs: 17, 53, 125 respectively,
SEQ ID NOs: 17, 52, 116 respectively,
SEQ ID NOs: 17, 53, 117 respectively,
SEQ ID NOs: 17, 52, 117 respectively,
SEQ ID NOs: 17, 40, 116 respectively,
SEQ ID NOs: 17, 53, 118 respectively,
SEQ ID NOs: 17, 54, 116 respectively,
SEQ ID NOs: 17, 55, 116 respectively,
SEQ ID NOs: 17, 54, 117 respectively,
SEQ ID NOs: 17, 40, 117 respectively,
SEQ ID NOs: 17, 55, 117 respectively,
SEQ ID NOs: 17, 54, 118 respectively,
SEQ ID NOs: 17, 40, 118 respectively,
SEQ ID NOs: 17, 55, 118 respectively,
SEQ ID NOs: 17, 55, 103 respectively,
SEQ ID NOs: 12, 55, 117 respectively, or
SEQ ID NOs: 12, 55, 118 respectively.

In some embodiments, the VH comprises an amino acid sequence having at least 80%, at least 85%, at least 90%, at least 95%, at least 98%, at least 99%, or 100% sequence identity to any one of SEQ ID NOs: 147-202.

In some embodiments, the antibody comprises an Fc region.

In some embodiments, the antibody comprises a heavy chain (HC), and wherein the HC comprises an amino acid sequence having at least 80%, at least 85%, at least 90%, at least 95%, at least 98%, at least 99%, or 100% sequence identity to any one of SEQ ID NOs: 205-260.

In some embodiments, the antibody does not comprise a light chain.

In some embodiments, the antibody comprises two heavy chains.

In some embodiments, the antibody is a chimeric antibody, a humanized antibody, or a human antibody.

In some embodiments, the antibody is of an isotype selected from the group consisting of IgG, IgA, IgM, IgE and IgD.

In some embodiments, the antibody is of a subtype selected from the group consisting of IgG1, IgG2, IgG3, and IgG4.

In some embodiments, the antigen binding fragment is selected from the group consisting of HCAb, VHH, nanobody, Fab, Fab′, F(ab′)₂, Fd, Fd′, and dAb.

In some embodiments, the antibody is a monoclonal antibody, a bi-specific or a multi-specific antibody.

In some embodiments, the antibody is monovalent, bivalent or multivalent.

In some embodiments, the antibody or antigen binding fragment is attached to a fluorescent label, radiolabel or cytotoxic agent.

In another aspect, the invention provides a bi-specific antibody, comprising the antibody or antigen-binding fragment thereof of the invention and a second antigen binding region specifically binding to a tumor associated antigen or an immune cell antigen; preferably, the second antigen binding region specifically binds to CD3.

In still another aspect, the invention provides a nucleic acid comprising a nucleotide sequence encoding the antibody or the antigen binding fragment thereof of the invention or the bi-specific antibody of the invention.

In yet another aspect, the invention provides a vector comprising the nucleic acid of the invention.

In another aspect, the invention provides a host cell comprising the nucleic acid of the invention or the vector of the invention.

In another aspect, the invention provides an antibody-drug conjugate (ADC), comprising the antibody or the antigen binding fragment thereof of the invention or the bi-specific antibody of the invention.

In still another aspect, the invention provides a pharmaceutical composition comprising (i) the antibody or the antigen binding fragment thereof of the invention, the bi-specific antibody of the invention, the nucleic acid of the invention, the vector of the invention, the host cell of the invention, or the antibody-drug conjugate of the invention; and (ii) a pharmaceutically acceptable carrier or excipient.

In some embodiments, the composition further comprises a second therapeutic agent selected from the group consisting of an antibody, a chemotherapeutic agent, a siRNA, an antisense oligonucleotide, a polypeptide, and a small molecule drug.

In another aspect, the invention provides a method of treating a cancer in a subject, comprising administering to the subject an effective amount of the antibody or the antigen binding fragment thereof of the invention, the bi-specific antibody of the invention, the nucleic acid of the invention, the vector of the invention, the host cell of the invention, the antibody-drug conjugate of the invention, or the pharmaceutical composition of the invention.

In some embodiments, the cancer is a ROR1 positive cancer, preferably selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

In some embodiments, the method further comprises administering to the subject a second therapeutic agent.

In some embodiments, the second therapeutic agent is selected from an antibody, a chemotherapeutic agent, a siRNA, an antisense oligonucleotide, a polypeptide, and a small molecule drug.

In another aspect, the invention provides use of the antibody or the antigen binding fragment thereof of the invention, the bi-specific antibody of the invention, the nucleic acid of the invention, the vector of the invention, the host cell of the invention, the antibody-drug conjugate of the invention, or the pharmaceutical composition of the invention in the manufacture of a medicament for treating a cancer in a subject.

In some embodiments, the cancer is a ROR1 positive cancer, preferably selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

In some embodiments, the medicament further comprises a second therapeutic agent, preferably the second therapeutic agent is selected from an antibody, a chemotherapeutic agent, a siRNA, an antisense oligonucleotide, a polypeptide, and a small molecule drug.

In some embodiments, the medicament is administered in combination with a second therapeutic agent, preferably the second therapeutic agent is selected from an antibody, a chemotherapeutic agent, a siRNA, an antisense oligonucleotide, a polypeptide, and a small molecule drug.

In another aspect, the invention provides the antibody or the antigen binding fragment thereof of the invention, the bi-specific antibody of the invention, the nucleic acid of the invention, the vector of the invention, the host cell of the invention, the antibody-drug conjugate of the invention, or the pharmaceutical composition of the invention for use in treating a cancer in a subject.

In some embodiments, the cancer is a ROR1 positive cancer, preferably selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

In some embodiments, the antibody or the antigen binding fragment thereof of the invention, the bi-specific antibody of the invention, the nucleic acid of the invention, the vector of the invention, the host cell of the invention, the antibody-drug conjugate of the invention, or the pharmaceutical composition of the invention is administered in combination with a second therapeutic agent, preferably the second therapeutic agent is selected from an antibody, a chemotherapeutic agent, a siRNA, an antisense oligonucleotide, a polypeptide, and a small molecule drug.

In another aspect, the invention provides a method for diagnosing ROR1 positive cancer in a subject comprising:
(a) obtaining a biological sample from the subject,
(b) contacting the sample with the antibody or the antigen binding fragment thereof of the invention, and
(c) detecting binding of the antibody to the sample,
wherein an increase in binding of the antibody or antigen binding fragment thereof to the sample as compared to binding of the antibody or antigen binging fragment thereof to a control sample identifies the subject as having a ROR1 positive cancer.

In another aspect, the invention provides a method for imaging a ROR1 positive cancer in a subject comprising:
(a) administering the antibody or antigen binding fragment thereof of the invention to the subject, wherein the antibody is conjugated to a detectable marker, and
(b) detecting the presence of the marker.

In some embodiments, the detectable marker is ¹¹¹In, and preferably the detection of the marker is by single-photon emission computed tomography. In some embodiments, the detectable marker is ⁸⁹Zr, and preferably the detection of the marker is by positron emission tomography.

### DESCRIPTION OF THE DRAWINGS

FIG. 1. The work flow of screening strategy and process for Single B-cell Cloning Screening.

FIG. 2. Binding of HCAb antibodies to CHO-K1-cynoROR1 cells.

FIG. 3. Binding of HCAb antibodies to PANC-1 cells.

FIG. 4. Binding of HCAb antibodies to HEK293T-hROR1 cells.

FIG. 5. Binding of HCAb antibodies to A549 cells.

FIG. 6. Binding of HCAb antibodies to hu-ROR1-ECD-his by ELISA.

FIG. 7. Binding of PR005340 variants of monovalent form (VH-Flag-His) to PANC-1 cells.

FIG. 8. Binding of PR005340 variants of bivalent form (HCAb) to PANC-1 cells.

FIG. 9. Binding of HCAb antibodies to hu-ROR2-his by ELISA.

FIG. 10. Internalization of HCAb antibodies on HEK293T-hROR1 cells by Ab-MMAF cytotoxicity method.

FIG. 11. Internalization of HCAb antibodies on PANC-1 cells by pHAb kit.

### SEQUENCE LISTING

The sequences of the heavy chain, the variable region of heavy chain, the CDRs of the heavy chain of anti-ROR1 HCAb antibodies of the invention are indicated in Tables 1-3 below. The sequences of the light chain, heavy chain, the variable region of light chain, the variable region of heavy chain, the CDRs of the light chain and heavy chain of reference antibody PR000374 are indicated in Table 4 below.

TABLE 1
Sequences of the heavy chain of of HCAb antibodies

| Clone | Heavy chain | SEQ ID NO |
|---|---|---|
| PR005337 | EVQLVESGGGLVKPGGSLRLSCAASGFIFSDYYMSWIRQAPGKGLEWVS | 205 |
| | YISSSGSTIHYADSVKGRFTVSRDNAKNSLYLQMNSLRTEDTAVYYCAR | |
| | DPPTSNSDWVSLHFDHWGQGTLVTVSSEPKSSDKTHTCPPCPAPELLGGP | |
| | SVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALHN | |
| | HYTQKSLSLSPGK | |
| PR005338 | EVQLVESGGGLVKPGGSLRLSCAASGFTFSDYYMSWIRQAPGKGLEWISYIS | 206 |
| | SSGTTIHYADSVKGRFTISRDNAKNSLYLQMNSLRTEDTAVYYCARDAPSSN | |
| | SDWVSLQFDYWGQGTLVTVSSEPKSSDKTHTCPPCPAPELLGGPSVFLFPPKP | |
| | KDTLMISRTPEVTCVVVDVSHEDPEVKFNWYVDGVEVHNAKTKPREEQYNS | |
| | TYRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| PR005339 | EVQLVESGGGLVKPGGSLKLSCAASGFTFSDYYMSWIRQAPGKGVEWIS | 207 |
| | YISNNGSTIHYADSVKGRFTISRDNAKNSLYLQMNSLRAEDTAVYYCAR | |
| | DFNNGWYEDFDYWGQGTLVTVSSEPKSSDKTHTCPPCPAPELLGGPSVF | |
| | LFPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKA | |
| | KGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHY | |
| | TQKSLSLSPGK | |
| PR005340 | EVQLVESGGGLVKPGGSLRLSCAASGFTFSDYYMSWIRQAPGKGLEWISYIS | 208 |
| | SSGSTIHYADSVKGRFTISRDNAKNSLYLQMNSLRAEDTAIYYCARDIPSSSS | |
| | DWVSLQFDYWGQGTLVTVSSEPKSSDKTHTCPPCPAPELLGGPSVFLFPPKP | |
| | KDTLMISRTPEVTCVVVDVSHEDPEVKFNWYVDGVEVHNAKTKPREEQYNS | |
| | TYRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| PR005341 | EVQLVESGGGLVKPGGSLRLSCVASGFTFSDYYMSWIRQAPGKGLEWIS | 209 |
| | YISSSGSSIYYAESVKGRFTISRDNAKNSLYLQMNRLRAEDTALYYCART | |
| | PPSSDNWYEDFDYWGQGALVTVSSEPKSSDKTHTCPPCPAPELLGGPSVF | |
| | LFPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKA | |
| | KGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHY | |
| | TQKSLSLSPGK | |
| PR005342 | EVQLVESGGGLVKPGGSLRLSCAASGFNFSDYYMSWIRQAPGKGLEWIS | 210 |
| | YISNSSSTIYYADSVKGRFTISRDNAKNSLYLQMSRLRAEDTAMYYCARD | |
| | TTNGWYEDFDYWGQGTLVTVSSEPKSSDKTHTCPPCPAPELLGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR005343 | EVQLVESGGGLVKPGGSLRLSCVASGFTFSDYYMSWIRQAPGKGMEWIS | 211 |
| | YISSSGSTKNYANSVKGRFTISRDNAKNSLYLQMNSLRAEDTAAYYCAR | |
| | VPPYNASWYEDFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR005344 | EVQLVESGGGLVKPGGSLRLSCAASGFTFSDYYMSWIRQAPGKGLEWIS | 212 |
| | YISNSSSSISYANSVKGRFTVSRDNAKNSLYLQMNSLRAEDTALYYCARS | |
| | PRGAFYEDFDYWGQGTLVTVSSEPKSSDKTHTCPPCPAPELLGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR005345 | EVQLVESGGGLVKPGGSLRLSCAASGFTFSDYYMSWIRQAPGKGLEWIA | 213 |
| | YISSSGSTIIIYSDSVKGRFTISRDNAKNSLYLQMNSLRAEDTAVYYCARD | |
| | TPSSSSDWVSLQFDYWGQGTPVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR005346 | EVQLVESGGGLVKPGGSLRLSCAASGFTLSDYYMSWIRQAPGKGLEWVS | 214 |
| | NISKNGFTIYYAESVKGRFTVSRDNAKNSLYLQMNSLRAEDTAIYYCAR | |
| | DSSGWYSEFDYWGQGTLVTVSSEPKSSDKTHTCPPCPAPELLGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR005347 | EVQLVESGGGLVKPGGSLRLSCVTSGFTFSDYYMSWIRQAPGKGMEWIS | 215 |
| | YISTTGSTKNYANSVKGRFTISRDNAKSSLYLQMNSLRAEDTAAYYCAR | |
| | VPPSNASWYEDFDYWGQGTLVTVSSEPKSSDKTHTCPPCPAPELLGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| PR005348 | EVQLVESGGGLVKPGGSLRLSCAASGFTFSDYYMSWIRQTPGKGLEWVS | 216 |
| | YISRSGSTKYYADSVKGRFTISRDNAKNSLYLQMNSLRAEDTAVYYCAR | |
| | DAPSSNSDWVSLHFDHWGQGTLVTVSSEPKSSDKTHTCPPCPAPELLGGP | |
| | SVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALHN | |
| | HYTQKSLSLSPGK | |
| PR005349 | EVQLVESGGGLVKPGGSLRLSCAASGFTFSDYYMSWIRQAPGKGLEWIS | 217 |
| | YISNSSSSISYANSVKGRFTVSRDNAKNSLYLQMNSLRAEDTALYYCARS | |
| | PRSAFYEDFDYWGQGTLVTVSSEPKSSDKTHTCPPCPAPELLGGPSVFLFP | |
| | PKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKG | |
| | QPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQ | |
| | KSLSLSPGK | |
| PR005350 | EVQLVESGGGLVKPGGSLRLSCAASGFTLSDSQMSWIRQAPGKGVEWVS | 218 |
| | YISSSGNTIYYGDSVKGRFTISRDNAKNSLFLQMNSLRAEDTAVYYCARV | |
| | PPSSSNWYEDFDIWGQGTLVTVSSEPKSSDKTHTCPPCPAPELLGGPSVFL | |
| | FPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR005351 | QVQLVESGGGLVKPGGSLRLSCAASGFKLSDFQMSWIRQAPGKGLEWV | 219 |
| | AYIDTNGSTRYYAESVKGRFTLSRDNVKNSLNLQMNGLRAEDTALYYC | |
| | ARIPSYTSSWYEDFDHWGQGTLVTVSSEPKSSDKTHTCPPCPAPELLGGP | |
| | SVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALHN | |
| | IIYTQKSLSLSPGK | |
| PR005352 | EVQLVESGGGLVKPGGSLRLSCAASGFTFSDYYMSWIRQAPGKGLEWIS | 220 |
| | YISSSGGSIYYADSVKGRFTISRDNAKNSLYLQMNRLRAEDTALYYCART | |
| | PPSSNNWYEDFDYWGQGALVTVSSEPKSSDKTHTCPPCPAPELLGGPSVF | |
| | LFPPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISKA | |
| | KGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHY | |
| | TQKSLSLSPGK | |
| PR303125 | EVQLLESGGGLVQPGGSLRLSCAASGFTFSSHAMSWVRQAPGKGLEWV | 253 |
| | SAISGSGDSTHYAASVKGRFTISRDNSKNTLYLQMNSLRAEDTAVYYCET | |
| | LLRFLESLGNDGFKIWGQGTMVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR303189 | EVQLVESGGGLVKPGGSLRLSCAASGFNLSDSYMSWIRQAPGKGLEWVS | 257 |
| | CISSSGSTIYYADSVKGRFTVSRDNAKNSLYLQMNHLRAEDTALYYCAR | |
| | DCVIGIRDDSDIWGQGTMVTVSSEPKSSDKTHTCPPCPAPELLGGPSVFLF | |
| | PPKPKDTLMISRTPEVTCVVVDVSIIEDPEVKFNWYVDGVEVIINAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR303191 | EVQLLESGGGLVQPGGSLRLSCAASGFIFGSYAMSWVRQAPGKGLEWVS | 258 |
| | GISGTGGNTYYADSVKGRFTISRDNSKNTLYLQMNSLRAEDTAVYFCER | |
| | GITIHGVVIIPPDYRGQGTLVTVSSEPKSSDKTHTCPPCPAPELLGGPSVFL | |
| | FPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR303199 | EVQLVESGGGLVKPGGSLRLSCAASGFTFSDYYMSWIRQAPGKGLEWIA | 259 |
| | YISSSGSTIYYADSVKGRFTISRDNAKNSLYLQMNSLRAEDTAIYYCARE | |
| | YYGSENYDHFDYWGQGTLVTVSSEPKSSDKTHTCPPCPAPELLGGPSVFL | |
| | FPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR303201 | EVQLLESGGGLVQPGGSLRLSCAASGFTFSGNAMSWVRQAPGKGLEWV | 260 |
| | SAISGSGDKTYYAASVKGRFTISRDNSNNTVYLQMNSLRAEDTAVYYCE | |
| | KGAFRTTMDYWGQGTLVTVSSEPKSSDKTHTCPPCPAPELLGGPSVFLFP | |
| | PKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKG | |
| | QPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQ | |
| | KSLSLSPGK | |
| PR303145 | QVQLVESGGGLVKPGGSLRISCAASGFTFSNYNMSWIRQAPGKGVEWVS | 254 |
| | HISGSGRTIYYAESVKGRFTISRDNAKNSLYLQMNSLRAEDTAMYYCAR | |
| | DLSSGWYEDFDYWGQGTLVTVSSEPKSSDKTHTCPPCPAPELLGGPSVFL | |
| | FPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR303147 | EVQLVESGGGLVQPGGSLRLSCAASGFTFSNFWMYWVRQAPGKGLVW | 255 |
| | VSRINSDGSSTNYADSVKGRFTISRDNAKNTLYLQMNSLRVEDTAVYSC | |
| | AREGSGWYEDFDYWGQGTLVTVSSEPKSSDKTHTCPPCPAPELLGGPSV | |
| | FLFPPKPKDTLMISRTPEVTCVVVDVSHEDPEVKFNWYVDGVEVHNAKT | |
| | KPREEQYNSTYRVVSVLTVLIIQDWLNGKEYKCKVSNKALPAPIEKTISK | |
| | AKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| PR303155 | EVQLVESGGGLVKPGGSLRLSCAASGFTLSDYYMSWIRQAPGKGLEWIS | 256 |
| | YISSNGSTIYYADSVKGRFTISRDNARNSLYLQMNSLRAEDTALYYCARD | |
| | VSSGWYEDFDYWGQGTLVTVSSEPKSSDKTHTCPPCPAPELLGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAK | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| PR009810 | EVQLVESGGGLVKPGGSLRLSCAASGFTFSDFYMSWIRQAPGKGLEWIS | 238 |
| | YISSSGSWIHYAHSVKGRFTISRDNAKNSLYLQMNSLRAEDTAIYYCARD | |
| | MPSSSEDWVDLQFDYWGQGTLVTVSSQGGSDYKDDDDKASHHHHHH | |
| PR009811 | EVQLVESGGGLVKPGGSLRLSCAASGFTFSDFYMSWIRQAPGKGLEWIS | 239 |
| | YISSSGSWIHYSGSVKGRFTISRDNAKNSLYLQMNSLRAEDTAIYYCARD | |
| | MPSSSSDWVDLQFDYWGQGTLVTVSSQGGSDYKDDDDKASHHHHHH | |
| PR009812 | EVQLVESGGGLVKPGGSLRLSCAASGFTFSDFYMSWIRQAPGKGLEWIS | 240 |
| | YISSSGSWIHYSHSVKGRFTISRDNAKNSLYLQMNSLRAEDTAIYYCARD | |
| | MPSSSEDWVDLQFDYWGQGTLVTVSSQGGSDYKDDDDKASHHHHHH | |
| PR009813 | EVQLVESGGGLVKPGGSLRLSCAASGFTFSDFYMSWIRQAPGKGLEWIS | 241 |
| | YISSSGSWIHYSSSVKGRFTISRDNAKNSLYLQMNSLRAEDTAIYYCARD | |
| | MPSSSIDWVELQFDYWGQGTLVTVSSQGGSDYKDDDDKASHHHHHH | |
| PR009814 | EVQLVESGGGLVKPGGSLRLSCAASGFTFSDFYMSWIRQAPGKGLEWIS | 242 |
| | YISSSGSWIIIYSPSVKGRFTISRDNAKNSLYLQMNSLRAEDTAIYYCARD | |
| | MPSSSKDWVDLQFDYWGQGTLVTVSSQGGSDYKDDDDKASHHHHHH | |
| PR009815 | EVQLVESGGGLVKPGGSLRLSCAASGFTFSDFYMSWIRQAPGKGLEWIS | 243 |
| | YISSSGSWIHYSPSVKGRFTISRDNAKNSLYLQMNSLRAEDTAIYYCARD | |
| | MPSSSIDWVDLQFDYWGQGTLVTVSSQGGSDYKDDDDKASHHHHHH | |
| PR009816 | EVQLVESGGGLVKPGGSLRLSCAASGFTFSDFYMSWIRQAPGKGLEWIS | 244 |
| | YISSSGSWIHYSASVKGRFTISRDNAKNSLYLQMNSLRAEDTAIYYCARD | |
| | MPSSSIDWVELQFDYWGQGTLVTVSSQGGSDYKDDDDKASHHHHHH | |
| PR009817 | EVQLVESGGGLVKPGGSLRLSCAASGFTFSDFYMSWIRQAPGKGLEWIS | 245 |
| | YISSSGSWIHYSTSVKGRFTISRDNAKNSLYLQMNSLRAEDTAIYYCARD | |
| | MPSSSSDWVDLQFDYWGQGTLVTVSSQGGSDYKDDDDKASHHHHHH | |
| PR009818 | EVQLVESGGGLVKPGGSLRLSCAASGFTFSDFYMSWIRQAPGKGLEWIS | 246 |
| | YISSSGSWIHYSSSVKGRFTISRDNAKNSLYLQMNSLRAEDTAIYYCARD | |
| | MPSSSEDWVELQFDYWGQGTLVTVSSQGGSDYKDDDDKASHHHHHH | |
| PR009819 | EVQLVESGGGLVKPGGSLRLSCAASGFTFSDFYMSWIRQAPGKGLEWIS | 247 |
| | YISSSGSWIHYSSSVKGRFTISRDNAKNSLYLQMNSLRAEDTAIYYCARD | |
| | MPSSSSDWVDLQFDYWGQGTLVTVSSQGGSDYKDDDDKASHHHHHH | |
| PR009820 | EVQLVESGGGLVKPGGSLRLSCAASGFTFSDFYMSWIRQAPGKGLEWIS | 248 |
| | YISSSGSWIHYAGSVKGRFTISRDNAKNSLYLQMNSLRAEDTAIYYCARD | |
| | MPSSSSDWVELQFDYWGQGTLVTVSSQGGSDYKDDDDKASHHHHHH | |
| PR009821 | EVQLVESGGGLVKPGGSLRLSCAASGFTFSDFYMSWIRQAPGKGLEWIS | 249 |
| | YISSSGSWIHYSSSVKGRFTISRDNAKNSLYLQMNSLRAEDTAIYYCARD | |
| | MPSSSEDWVMLQFDYWGQGTLVTVSSQGGSDYKDDDDKASHHHHHH | |
| PR009822 | EVQLVESGGGLVKPGGSLRLSCAASGFTFSDFYMSWIRQAPGKGLEWIS | 250 |
| | YISSSGSWIHYATSVKGRFTISRDNAKNSLYLQMNSLRAEDTAIYYCARD | |
| | MPSSSIDWVDLQFDYWGQGTLVTVSSQGGSDYKDDDDKASHHHHHH | |
| PR009823 | EVQLVESGGGLVKPGGSLRLSCAASGFTFSDFYMSWIRQAPGKGLEWIS | 25 |
| | YISSSGSWIHYASSVKGRFTISRDNAKNSLYLQMNSLRAEDTAIYYCARD | |
| | MPSSSSDWVDLQFDYWGQGTLVTVSSQGGSDYKDDDDKASHHHHHH | |
| PR009824 | EVQLVESGGGLVKPGGSLRLSCAASGFTFSDFYMSWIRQAPGKGLEWIS | 252 |
| | YISSSGSWIHYSTSVKGRFTISRDNAKNSLYLQMNSLRAEDTAIYYCARD | |
| | MPSSSIDWVDLQFDYWGQGTLVTVSSQGGSDYKDDDDKASHHHHHH | |
| PR007408 | EVQLVESGGGLVKPGGSLRLSCAASGFTFSDFYMSWIRQAPGKGLEWIS | 221 |
| | YISSSGSYIHYADSVKGRFTISRDNAKNSLYLQMNSLRAEDTAIYYCARD | |
| | MPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007409 | EVQLVESGGGLVKPGGSLRLSCAASGFTFSDFYMSWIRQAPGKGLEWIS | 222 |
| | YISSSGSWIHYADSVKGRFTISRDNAKNSLYLQMNSLRAEDTAIYYCARD | |
| | IPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007410 | EVQLVESGGGLVKPGGSLRLSCAASGFTFSDFYMSWIRQAPGKGLEWIS | 223 |
| | YISSSGSYIHYADSVKGRFTISRDNAKNSLYLQMNSLRAEDTAIYYCARDI | |
| | PSSSSDWVDLQFDYWGQGTLVTVSSEPKSSDKTHTCPPCPAPELLGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| PR007411 | EVQLVESGGGLVKPGGSLRLSCAASGFTFSDFYMSWIRQAPGKGLEWIS | 224 |
| | YISSSGSTIHYADSVKGRFTISRDNAKNSLYLQMNSLRAEDTAIYYCARD | |
| | MPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007412 | EVQLVESGGGLVKPGGSLRLSCAASGFTFSDFYMSWIRQAPGKGLEWIS | 225 |
| | YISSSGSWIHYADSVKGRFTISRDNAKNSLYLQMNSLRAEDTAIYYCARD | |
| | MPSSSSDWVSLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007413 | EVQLVESGGGLVKPGGSLRLSCAASGFTFSDFYMSWIRQAPGKGLEWIS | 226 |
| | YINTRGSPIGYADSVKGRFTISRDNAKNSLYLQMNSLRAEDTAIYYCARD | |
| | MPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007414 | EVQLVESGGGLVKPGGSLRLSCAASGFTFSDFYMSWIRQAPGKGLEWIS | 227 |
| | YISSSGSTRYYADSVKGRFTISRDNAKNSLYLQMNSLRAEDTAIYYCARD | |
| | MPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007415 | EVQLVESGGGLVKPGGSLRLSCAASGFTFSDFYMSWIRQAPGKGLEWIS | 228 |
| | YIDSSGRPLAYADSVKGRFTISRDNAKNSLYLQMNSLRAEDTAIYYCARD | |
| | MPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007416 | EVQLVESGGGLVKPGGSLRLSCAASGFTFSDFYMSWIRQAPGKGLEWIS | 229 |
| | YINTRGSPIGYADSVKGRFTISRDNAKNSLYLQMNSLRAEDTAIYYCARD | |
| | IPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007417 | EVQLVESGGGLVKPGGSLRLSCAASGFTFSDFYMSWIRQAPGKGLEWIS | 230 |
| | YISSSGSTRYYADSVKGRFTISRDNAKNSLYLQMNSLRAEDTAIYYCARD | |
| | IPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007418 | EVQLVESGGGLVKPGGSLRLSCAASGFTFSDFYMSWIRQAPGKGLEWIS | 231 |
| | YIDSSGRPLAYADSVKGRFTISRDNAKNSLYLQMNSLRAEDTAIYYCARD | |
| | IPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLIIQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007419 | EVQLVESGGGLVKPGGSLRLSCAASGFTFSDFYMSWIRQAPGKGLEWIS | 232 |
| | YINTRGSPIGYADSVKGRFTISRDNAKNSLYLQMNSLRAEDTAIYYCARD | |
| | MPSSSSDWVSLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007420 | EVQLVESGGGLVKPGGSLRLSCAASGFTFSDFYMSWIRQAPGKGLEWIS | 233 |
| | YISSSGSTRYYADSVKGRFTISRDNAKNSLYLQMNSLRAEDTAIYYCARD | |
| | MPSSSSDWVSLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007421 | EVQLVESGGGLVKPGGSLRLSCAASGFTFSDFYMSWIRQAPGKGLEWIS | 234 |
| | YIDSSGRPLAYADSVKGRFTISRDNAKNSLYLQMNSLRAEDTAIYYCARD | |
| | MPSSSSDWVSLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007422 | EVQLVESGGGLVKPGGSLRLSCAASGFTFSDFYMSWIRQAPGKGLEWIS | 235 |
| | YIDSSGRPLAYADSVKGRFTISRDNAKNSLYLQMNSLRAEDTAIYYCARD | |
| | IPSSSSDWVSLQFDYWGQGTLVTVSSEPKSSDKTHTCPPCPAPELLGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| PR007423 | EVQLVESGGGLVKPGGSLRLSCAASGFTFSDYYMSWIRQAPGKGLEWIS | 236 |
| | YIDSSGRPLAYADSVKGRFTISRDNAKNSLYLQMNSLRAEDTAIYYCARD | |
| | IPSSSSDWVDLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLIIQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |
| PR007424 | EVQLVESGGGLVKPGGSLRLSCAASGFTFSDYYMSWIRQAPGKGLEWIS | 237 |
| | YIDSSGRPLAYADSVKGRFTISRDNAKNSLYLQMNSLRAEDTAIYYCARD | |
| | MPSSSSDWVSLQFDYWGQGTLVTVSSEPKSSDKTHTCPPCPAPELLGGPS | |
| | VFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNH | |
| | YTQKSLSLSPGK | |

TABLE 2
Sequences of the heavy chain variable region of HCAb antibodies

| Clone | Heavy chain variable region | SEQ ID NO |
|---|---|---|
| PR005337 | EVQLVESGGGLVKPGGSLRLSCAASGFIFSDYYMSWIRQAPGKGLEWV | 147 |
| | SYISSSGSTIHYADSVKGRFTVSRDNAKNSLYLQMNSLRTEDTAVYYCA | |
| | RDPPTSNSDWVSLHFDHWGQGTLVTVSS | |
| PR005338 | EVQLVESGGGLVKPGGSLRLSCAASGFTFSDYYMSWIRQAPGKGLEWI | 148 |
| | SYISSSGTTIHYADSVKGRFTISRDNAKNSLYLQMNSLRTEDTAVYYCA | |
| | RDAPSSNSDWVSLQFDYWGQGTLVTVSS | |
| PR005339 | EVQLVESGGGLVKPGGSLKLSCAASGFTFSDYYMSWIRQAPGKGVEWI | 149 |
| | SYISNNGSTIHYADSVKGRFTISRDNAKNSLYLQMNSLRAEDTAVYYC | |
| | ARDFNNGWYEDFDYWGQGTLVTVSS | |
| PR005340 | EVQLVESGGGLVKPGGSLRLSCAASGFTFSDYYMSWIRQAPGKGLEWI | 150 |
| | SYISSSGSTIHYADSVKGRFTISRDNAKNSLYLQMNSLRAEDTAIYYCAR | |
| | DIPSSSSDWVSLQFDYWGQGTLVTVSS | |
| PR005341 | EVQLVESGGGLVKPGGSLRLSCVASGFTFSDYYMSWIRQAPGKGLEWI | 151 |
| | SYISSSGSSIYYAESVKGRFTISRDNAKNSLYLQMNRLRAEDTALYYCA | |
| | RTPPSSDNWYEDFDYWGQGALVTVSS | |
| PR005342 | EVQLVESGGGLVKPGGSLRLSCAASGFNFSDYYMSWIRQAPGKGLEWI | 152 |
| | SYISNSSSTIYYADSVKGRFTISRDNAKNSLYLQMSRLRAEDTAMYYCA | |
| | RDTTNGWYEDFDYWGQGTLVTVSS | |
| PR005343 | EVQLVESGGGLVKPGGSLRLSCVASGFTFSDYYMSWIRQAPGKGMEWI | 153 |
| | SYISSSGSTKNYANSVKGRFTISRDNAKNSLYLQMNSLRAEDTAAYYC | |
| | ARVPPYNASWYEDFDYWGQGTLVTVSS | |
| PR005344 | EVQLVESGGGLVKPGGSLRLSCAASGFTFSDYYMSWIRQAPGKGLEWI | 154 |
| | SYISNSSSSISYANSVKGRFTVSRDNAKNSLYLQMNSLRAEDTALYYCA | |
| | RSPRGAFYEDFDYWGQGTLVTVSS | |
| PR005345 | EVQLVESGGGLVKPGGSLRLSCAASGFTFSDYYMSWIRQAPGKGLEWI | 155 |
| | AYISSSGSTIHYSDSVKGRFTISRDNAKNSLYLQMNSLRAEDTAVYYCA | |
| | RDTPSSSSDWVSLQFDYWGQGTPVTVSS | |
| PR005346 | EVQLVESGGGLVKPGGSLRLSCAASGFTLSDYYMSWIRQAPGKGLEW | 156 |
| | VSNISKNGFTIYYAESVKGRFTVSRDNAKNSLYLQMNSLRAEDTAIYYC | |
| | ARDSSGWYSEFDYWGQGTLVTVSS | |
| PR005347 | EVQLVESGGGLVKPGGSLRLSCVTSGFTFSDYYMSWIRQAPGKGMEWI | 157 |
| | SYISTTGSTKNYANSVKGRFTISRDNAKSSLYLQMNSLRAEDTAAYYC | |
| | ARVPPSNASWYEDFDYWGQGTLVTVSS | |
| PR005348 | EVQLVESGGGLVKPGGSLRLSCAASGFTFSDYYMSWIRQTPGKGLEWV | 158 |
| | SYISRSGSTKYYADSVKGRFTISRDNAKNSLYLQMNSLRAEDTAVYYC | |
| | ARDAPSSNSDWVSLHFDHWGQGTLVTVSS | |
| PR005349 | EVQLVESGGGLVKPGGSLRLSCAASGFTFSDYYMSWIRQAPGKGLEWI | 159 |
| | SYISNSSSSISYANSVKGRFTVSRDNAKNSLYLQMNSLRAEDTALYYCA | |
| | RSPRSAFYEDFDYWGQGTLVTVSS | |
| PR005350 | EVQLVESGGGLVKPGGSLRLSCAASGFTLSDSQMSWIRQAPGKGVEW | 160 |
| | VSYISSSGNTIYYGDSVKGRFTISRDNAKNSLFLQMNSLRAEDTAVYYC | |
| | ARVPPSSSNWYEDFDIWGQGTLVTVSS | |
| PR005351 | QVQLVESGGGLVKPGGSLRLSCAASGFKLSDFQMSWIRQAPGKGLEW | 161 |
| | VAYIDTNGSTRYYAESVKGRFTLSRDNVKNSLNLQMNGLRAEDTALY | |
| | YCARIPSYTSSWYEDFDHWGQGTLVTVSS | |
| PR005352 | EVQLVESGGGLVKPGGSLRLSCAASGFTFSDYYMSWIRQAPGKGLEWI | 162 |
| | SYISSSGGSIYYADSVKGRFTISRDNAKNSLYLQMNRLRAEDTALYYCA | |
| | RTPPSSNNWYEDFDYWGQGALVTVSS | |
| PR303125 | EVQLLESGGGLVQPGGSLRLSCAASGFTFSSHAMSWVRQAPGKGLEW | 195 |
| | VSAISGSGDSTHYAASVKGRFTISRDNSKNTLYLQMNSLRAEDTAVYY | |
| | CETLLRFLESLGNDGFKIWGQGTMVTVSS | |
| PR303189 | EVQLVESGGGLVKPGGSLRLSCAASGFNLSDSYMSWIRQAPGKGLEW | 199 |
| | VSCISSSGSTIYYADSVKGRFTVSRDNAKNSLYLQMNHLRAEDTALYY | |
| | CARDCVIGIRDDSDIWGQGTMVTVSS | |
| PR303191 | EVQLLESGGGLVQPGGSLRLSCAASGFIFGSYAMSWVRQAPGKGLEW | 200 |
| | VSGISGTGGNTYYADSVKGRFTISRDNSKNTLYLQMNSLRAEDTAVYF | |
| | CERGITIHGVVIIPPDYRGQGTLVTVSS | |
| PR303199 | EVQLVESGGGLVKPGGSLRLSCAASGFTFSDYYMSWIRQAPGKGLEWI | 201 |
| | AYISSSGSTIYYADSVKGRFTISRDNAKNSLYLQMNSLRAEDTAIYYCA | |
| | REYYGSENYDHFDYWGQGTLVTVSS | |
| PR303201 | EVQLLESGGGLVQPGGSLRLSCAASGFTFSGNAMSWVRQAPGKGLEW | 202 |
| | VSAISGSGDKTYYAASVKGRFTISRDNSNNTVYLQMNSLRAEDTAVYY | |
| | CEKGAFRTTMDYWGQGTLVTVSS | |
| PR303145 | QVQLVESGGGLVKPGGSLRISCAASGFTFSNYNMSWIRQAPGKGVEWV | 196 |
| | SHISGSGRTIYYAESVKGRFTISRDNAKNSLYLQMNSLRAEDTAMYYC | |
| | ARDLSSGWYEDFDYWGQGTLVTVSS | |
| PR303147 | EVQLVESGGGLVQPGGSLRLSCAASGFTFSNFWMYWVRQAPGKGLV | 197 |
| | WVSRINSDGSSTNYADSVKGRFTISRDNAKNTLYLQMNSLRVEDTAVY | |
| | SCAREGSGWYEDFDYWGQGTLVTVSS | |
| PR303155 | EVQLVESGGGLVKPGGSLRLSCAASGFTLSDYYMSWIRQAPGKGLEWI | 198 |
| | SYISSNGSTIYYADSVKGRFTISRDNARNSLYLQMNSLRAEDTALYYCA | |
| | RDVSSGWYEDFDYWGQGTLVTVSS | |
| PR009810 | EVQLVESGGGLVKPGGSLRLSCAASGFTFSDFYMSWIRQAPGKGLEWI | 180 |
| | SYISSSGSWIHYAHSVKGRFTISRDNAKNSLYLQMNSLRAEDTAIYYCA | |
| | RDMPSSSEDWVDLQFDYWGQGTLVTVSS | |
| PR009811 | EVQLVESGGGLVKPGGSLRLSCAASGFTFSDFYMSWIRQAPGKGLEWI | 181 |
| | SYISSSGSWIHYSGSVKGRFTISRDNAKNSLYLQMNSLRAEDTAIYYCA | |
| | RDMPSSSSDWVDLQFDYWGQGTLVTVSS | |
| PR009812 | EVQLVESGGGLVKPGGSLRLSCAASGFTFSDFYMSWIRQAPGKGLEWI | 182 |
| | SYISSSGSWIHYSHSVKGRFTISRDNAKNSLYLQMNSLRAEDTAIYYCA | |
| | RDMPSSSEDWVDLQFDYWGQGTLVTVSS | |
| PR009813 | EVQLVESGGGLVKPGGSLRLSCAASGFTFSDFYMSWIRQAPGKGLEWI | 183 |
| | SYISSSGSWIHYSSSVKGRFTISRDNAKNSLYLQMNSLRAEDTAIYYCAR | |
| | DMPSSSIDWVELQFDYWGQGTLVTVSS | |
| PR009814 | EVQLVESGGGLVKPGGSLRLSCAASGFTFSDFYMSWIRQAPGKGLEWI | 184 |
| | SYISSSGSWIHYSPSVKGRFTISRDNAKNSLYLQMNSLRAEDTAIYYCAR | |
| | DMPSSSKDWVDLQFDYWGQGTLVTVSS | |
| PR009815 | EVQLVESGGGLVKPGGSLRLSCAASGFTFSDFYMSWIRQAPGKGLEWI | 185 |
| | SYISSSGSWIHYSPSVKGRFTISRDNAKNSLYLQMNSLRAEDTAIYYCAR | |
| | DMPSSSIDWVDLQFDYWGQGTLVTVSS | |
| PR009816 | EVQLVESGGGLVKPGGSLRLSCAASGFTFSDFYMSWIRQAPGKGLEWI | 186 |
| | SYISSSGSWIHYSASVKGRFTISRDNAKNSLYLQMNSLRAEDTAIYYCA | |
| | RDMPSSSIDWVELQFDYWGQGTLVTVSS | |
| PR009817 | EVQLVESGGGLVKPGGSLRLSCAASGFTFSDFYMSWIRQAPGKGLEWI | 187 |
| | SYISSSGSWIHYSTSVKGRFTISRDNAKNSLYLQMNSLRAEDTAIYYCA | |
| | RDMPSSSSDWVDLQFDYWGQGTLVTVSS | |
| PR009818 | EVQLVESGGGLVKPGGSLRLSCAASGFTFSDFYMSWIRQAPGKGLEWI | 188 |
| | SYISSSGSWIHYSSSVKGRFTISRDNAKNSLYLQMNSLRAEDTAIYYCAR | |
| | DMPSSSEDWVELQFDYWGQGTLVTVSS | |
| PR009819 | EVQLVESGGGLVKPGGSLRLSCAASGFTFSDFYMSWIRQAPGKGLEWI | 189 |
| | SYISSSGSWIHYSSSVKGRFTISRDNAKNSLYLQMNSLRAEDTAIYYCAR | |
| | DMPSSSSDWVDLQFDYWGQGTLVTVSS | |
| PR009820 | EVQLVESGGGLVKPGGSLRLSCAASGFTFSDFYMSWIRQAPGKGLEWI | 190 |
| | SYISSSGSWIHYAGSVKGRFTISRDNAKNSLYLQMNSLRAEDTAIYYCA | |
| | RDMPSSSSDWVELQFDYWGQGTLVTVSS | |
| PR009821 | EVQLVESGGGLVKPGGSLRLSCAASGFTFSDFYMSWIRQAPGKGLEWI | 191 |
| | SYISSSGSWIHYSSSVKGRFTISRDNAKNSLYLQMNSLRAEDTAIYYCAR | |
| | DMPSSSEDWVMLQFDYWGQGTLVTVSS | |
| PR009822 | EVQLVESGGGLVKPGGSLRLSCAASGFTFSDFYMSWIRQAPGKGLEWI | 192 |
| | SYISSSGSWIHYATSVKGRFTISRDNAKNSLYLQMNSLRAEDTAIYYCA | |
| | RDMPSSSIDWVDLQFDYWGQGTLVTVSS | |
| PR009823 | EVQLVESGGGLVKPGGSLRLSCAASGFTFSDFYMSWIRQAPGKGLEWI | 193 |
| | SYISSSGSWIHYASSVKGRFTISRDNAKNSLYLQMNSLRAEDTAIYYCA | |
| | RDMPSSSSDWVDLQFDYWGQGTLVTVSS | |
| PR009824 | EVQLVESGGGLVKPGGSLRLSCAASGFTFSDFYMSWIRQAPGKGLEWI | 194 |
| | SYISSSGSWIHYSTSVKGRFTISRDNAKNSLYLQMNSLRAEDTAIYYCA | |
| | RDMPSSSIDWVDLQFDYWGQGTLVTVSS | |
| PR007408 | EVQLVESGGGLVKPGGSLRLSCAASGFTFSDFYMSWIRQAPGKGLEWI | 163 |
| | SYISSSGSYIHYADSVKGRFTISRDNAKNSLYLQMNSLRAEDTAIYYCA | |
| | RDMPSSSSDWVDLQFDYWGQGTLVTVSS | |
| PR007409 | EVQLVESGGGLVKPGGSLRLSCAASGFTFSDFYMSWIRQAPGKGLEWI | 164 |
| | SYISSSGSWIHYADSVKGRFTISRDNAKNSLYLQMNSLRAEDTAIYYCA | |
| | RDIPSSSSDWVDLQFDYWGQGTLVTVSS | |
| PR007410 | EVQLVESGGGLVKPGGSLRLSCAASGFTFSDFYMSWIRQAPGKGLEWI | 165 |
| | SYISSSGSYIHYADSVKGRFTISRDNAKNSLYLQMNSLRAEDTAIYYCA | |
| | RDIPSSSSDWVDLQFDYWGQGTLVTVSS | |
| PR007411 | EVQLVESGGGLVKPGGSLRLSCAASGFTFSDFYMSWIRQAPGKGLEWI | 166 |
| | SYISSSGSTIHYADSVKGRFTISRDNAKNSLYLQMNSLRAEDTAIYYCAR | |
| | DMPSSSSDWVDLQFDYWGQGTLVTVSS | |
| PR007412 | EVQLVESGGGLVKPGGSLRLSCAASGFTFSDFYMSWIRQAPGKGLEWI | 167 |
| | SYISSSGSWIHYADSVKGRFTISRDNAKNSLYLQMNSLRAEDTAIYYCA | |
| | RDMPSSSSDWVSLQFDYWGQGTLVTVSS | |
| PR007413 | EVQLVESGGGLVKPGGSLRLSCAASGFTFSDFYMSWIRQAPGKGLEWI | 168 |
| | SYINTRGSPIGYADSVKGRFTISRDNAKNSLYLQMNSLRAEDTAIYYCA | |
| | RDMPSSSSDWVDLQFDYWGQGTLVTVSS | |
| PR007414 | EVQLVESGGGLVKPGGSLRLSCAASGFTFSDFYMSWIRQAPGKGLEWI | 169 |
| | SYISSSGSTRYYADSVKGRFTISRDNAKNSLYLQMNSLRAEDTAIYYCA | |
| | RDMPSSSSDWVDLQFDYWGQGTLVTVSS | |
| PR007415 | EVQLVESGGGLVKPGGSLRLSCAASGFTFSDFYMSWIRQAPGKGLEWI | 170 |
| | SYIDSSGRPLAYADSVKGRFTISRDNAKNSLYLQMNSLRAEDTAIYYCA | |
| | RDMPSSSSDWVDLQFDYWGQGTLVTVSS | |
| PR007416 | EVQLVESGGGLVKPGGSLRLSCAASGFTFSDFYMSWIRQAPGKGLEWI | 171 |
| | SYINTRGSPIGYADSVKGRFTISRDNAKNSLYLQMNSLRAEDTAIYYCA | |
| | RDIPSSSSDWVDLQFDYWGQGTLVTVSS | |
| PR007417 | EVQLVESGGGLVKPGGSLRLSCAASGFTFSDFYMSWIRQAPGKGLEWI | 172 |
| | SYISSSGSTRYYADSVKGRFTISRDNAKNSLYLQMNSLRAEDTAIYYCA | |
| | RDIPSSSSDWVDLQFDYWGQGTLVTVSS | |
| PR007418 | EVQLVESGGGLVKPGGSLRLSCAASGFTFSDFYMSWIRQAPGKGLEWI | 173 |
| | SYIDSSGRPLAYADSVKGRFTISRDNAKNSLYLQMNSLRAEDTAIYYCA | |
| | RDIPSSSSDWVDLQFDYWGQGTLVTVSS | |
| PR007419 | EVQLVESGGGLVKPGGSLRLSCAASGFTFSDFYMSWIRQAPGKGLEWI | 174 |
| | SYINTRGSPIGYADSVKGRFTISRDNAKNSLYLQMNSLRAEDTAIYYCA | |
| | RDMPSSSSDWVSLQFDYWGQGTLVTVSS | |
| PR007420 | EVQLVESGGGLVKPGGSLRLSCAASGFTFSDFYMSWIRQAPGKGLEWI | 175 |
| | SYISSSGSTRYYADSVKGRFTISRDNAKNSLYLQMNSLRAEDTAIYYCA | |
| | RDMPSSSSDWVSLQFDYWGQGTLVTVSS | |
| PR007421 | EVQLVESGGGLVKPGGSLRLSCAASGFTFSDFYMSWIRQAPGKGLEWI | 176 |
| | SYIDSSGRPLAYADSVKGRFTISRDNAKNSLYLQMNSLRAEDTAIYYCA | |
| | RDMPSSSSDWVSLQFDYWGQGTLVTVSS | |
| PR007422 | EVQLVESGGGLVKPGGSLRLSCAASGFTFSDFYMSWIRQAPGKGLEWI | 177 |
| | SYIDSSGRPLAYADSVKGRFTISRDNAKNSLYLQMNSLRAEDTAIYYCA | |
| | RDIPSSSSDWVSLQFDYWGQGTLVTVSS | |
| PR007423 | EVQLVESGGGLVKPGGSLRLSCAASGFTFSDYYMSWIRQAPGKGLEWI | 178 |
| | SYIDSSGRPLAYADSVKGRFTISRDNAKNSLYLQMNSLRAEDTAIYYCA | |
| | RDIPSSSSDWVDLQFDYWGQGTLVTVSS | |
| PR007424 | EVQLVESGGGLVKPGGSLRLSCAASGFTFSDYYMSWIRQAPGKGLEWI | 179 |
| | SYIDSSGRPLAYADSVKGRFTISRDNAKNSLYLQMNSLRAEDTAIYYCA | |
| | RDMPSSSSDWVSLQFDYWGQGTLVTVSS | |

TABLE 3
Sequences of the heavy chain CDRs 1-3 of HCAb antibodies (Chothia numbering
system)

| | Heavy chain | SEQ ID | Heavy chain | SEQ ID | Heavy chain | SEQ ID |
|---|---|---|---|---|---|---|
| Clone | CDR1 | NO | CDR2 | NO | CDR3 | NO |
| PR005337 | GFIFSDY | 11 | SSSGST | 40 | DPPTSNSDWVSLHFDH | 100 |
| PR005338 | GFTFSDY | 12 | SSSGTT | 41 | DAPSSNSDWVSLQFDY | 101 |
| PR005339 | GFTFSDY | 12 | SNNGST | 42 | DFNNGWYEDFDY | 102 |
| PR005340 | GFTFSDY | 12 | SSSGST | 40 | DIPSSSSDWVSLQFDY | 103 |
| PR005341 | GFTFSDY | 12 | SSSGSS | 43 | TPPSSDNWYEDFDY | 104 |
| PR005342 | GFNFSDY | 13 | SNSSST | 44 | DTTNGWYEDFDY | 105 |
| PR005343 | GFTFSDY | 12 | SSSGST | 40 | VPPYNASWYEDFDY | 106 |
| PR005344 | GFTFSDY | 12 | SNSSSS | 45 | SPRGAFYEDFDY | 107 |
| PR005345 | GFTFSDY | 12 | SSSGST | 40 | DTPSSSSDWVSLQFDY | 108 |
| PR005346 | GFTLSDY | 14 | SKNGFT | 46 | DSSGWYSEFDY | 109 |
| PR005347 | GFTFSDY | 12 | STTGST | 47 | VPPSNASWYEDFDY | 110 |
| PR005348 | GFTFSDY | 12 | SRSGST | 48 | DAPSSNSDWVSLHFDH | 111 |
| PR005349 | GFTFSDY | 12 | SNSSSS | 45 | SPRSAFYEDFDY | 112 |
| PR005350 | GFTLSDS | 15 | SSSGNT | 49 | VPPSSSNWYEDFDI | 113 |
| PR005351 | GFKLSDF | 16 | DTNGST | 50 | IPSYTSSWYEDFDH | 114 |
| PR005352 | GFTFSDY | 12 | SSSGGS | 51 | TPPSSNNWYEDFDY | 115 |
| PR303125 | GFTFSSH | 18 | SGSGDS | 56 | LLRFLESLGNDGFKI | 126 |
| PR303189 | GFNLSDS | 21 | SSSGST | 40 | DCVIGIRDDSDI | 130 |
| PR303191 | GFIFGSY | 22 | SGTGGN | 60 | GITIHGVVIIPPDY | 131 |
| PR303199 | GFTFSDY | 12 | SSSGST | 40 | EYYGSENYDHFDY | 132 |
| PR303201 | GFTFSGN | 23 | SGSGDK | 61 | GAFRTTMDY | 133 |
| PR303145 | GFTFSNY | 19 | SGSGRT | 57 | DLSSGWYEDFDY | 127 |
| PR303147 | GFTFSNF | 20 | NSDGSS | 58 | EGSGWYEDFDY | 128 |
| PR303155 | GFTLSDY | 14 | SSNGST | 59 | DVSSGWYEDFDY | 129 |
| PR009810 | GFTFSDF | 17 | SSSGSW | 53 | DMPSSSEDWVDLQFDY | 119 |
| PR009811 | GFTFSDF | 17 | SSSGSW | 53 | DMPSSSSDWVDLQFDY | 116 |
| PR009812 | GFTFSDF | 17 | SSSGSW | 53 | DMPSSSEDWVDLQFDY | 119 |
| PR009813 | GFTFSDF | 17 | SSSGSW | 53 | DMPSSSIDWVELQFDY | 120 |
| PR009814 | GFTFSDF | 17 | SSSGSW | 53 | DMPSSSKDWVDLQFDY | 121 |
| PR009815 | GFTFSDF | 17 | SSSGSW | 53 | DMPSSSIDWVDLQFDY | 122 |
| PR009816 | GFTFSDF | 17 | SSSGSW | 53 | DMPSSSIDWVELQFDY | 120 |
| PR009817 | GFTFSDF | 17 | SSSGSW | 53 | DMPSSSSDWVDLQFDY | 116 |
| PR009818 | GFTFSDF | 17 | SSSGSW | 53 | DMPSSSEDWVELQFDY | 123 |
| PR009819 | GFTFSDF | 17 | SSSGSW | 53 | DMPSSSSDWVDLQFDY | 116 |
| PR009820 | GFTFSDF | 17 | SSSGSW | 53 | DMPSSSSDWVELQFDY | 124 |
| PR009821 | GFTFSDF | 17 | SSSGSW | 53 | DMPSSSEDWVMLQFDY | 125 |
| PR009822 | GFTFSDF | 17 | SSSGSW | 53 | DMPSSSIDWVDLQFDY | 122 |
| PR009823 | GFTFSDF | 17 | SSSGSW | 53 | DMPSSSSDWVDLQFDY | 116 |
| PR009824 | GFTFSDF | 17 | SSSGSW | 53 | DMPSSSIDWVDLQFDY | 122 |
| PR007408 | GFTFSDF | 17 | SSSGSY | 52 | DMPSSSSDWVDLQFDY | 116 |
| PR007409 | GFTFSDF | 17 | SSSGSW | 53 | DIPSSSSDWVDLQFDY | 117 |
| PR007410 | GFTFSDF | 17 | SSSGSY | 52 | DIPSSSSDWVDLQFDY | 117 |
| PR007411 | GFTFSDF | 17 | SSSGST | 40 | DMPSSSSDWVDLQFDY | 116 |
| PR007412 | GFTFSDF | 17 | SSSGSW | 53 | DMPSSSSDWVSLQFDY | 118 |
| PR007413 | GFTFSDF | 17 | NTRGSP | 54 | DMPSSSSDWVDLQFDY | 116 |
| PR007414 | GFTFSDF | 17 | SSSGST | 40 | DMPSSSSDWVDLQFDY | 116 |
| PR007415 | GFTFSDF | 17 | DSSGRP | 55 | DMPSSSSDWVDLQFDY | 116 |
| PR007416 | GFTFSDF | 17 | NTRGSP | 54 | DIPSSSSDWVDLQFDY | 117 |
| PR007417 | GFTFSDF | 17 | SSSGST | 40 | DIPSSSSDWVDLQFDY | 117 |
| PR007418 | GFTFSDF | 17 | DSSGRP | 55 | DIPSSSSDWVDLQFDY | 117 |
| PR007419 | GFTFSDF | 17 | NTRGSP | 54 | DMPSSSSDWVSLQFDY | 118 |
| PR007420 | GFTFSDF | 17 | SSSGST | 40 | DMPSSSSDWVSLQFDY | 118 |
| PR007421 | GFTFSDF | 17 | DSSGRP | 55 | DMPSSSSDWVSLQFDY | 118 |
| PR007422 | GFTFSDF | 17 | DSSGRP | 55 | DIPSSSSDWVSLQFDY | 103 |
| PR007423 | GFTFSDY | 12 | DSSGRP | 55 | DIPSSSSDWVDLQFDY | 117 |
| PR007424 | GFTFSDY | 12 | DSSGRP | 55 | DMPSSSSDWVSLQFDY | 118 |

TABLE 4
Sequences of reference antibody PR000374

| | Sequence | SEQ ID NO |
|---|---|---|
| Heavy | EVQLVESGGGLVQPGRSLRLSCTASGSDINDYPITWVRQAPGQGLEWIG | 204 |
| Chain | FINSGGSTWYASWVKGRFTISRDDSKSIAYLQMNSLKTEDTAVYYCAR | |
| | GYSTYYRDFNIWGQGTLVTVSSASTKGPSVFPLAPSSKSTSGGTAALGC | |
| | LVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPELLGGPSVFL | |
| | FPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALH | |
| | NHYTQKSLSLSPGK | |
| Heavy chain | EVQLVESGGGLVQPGRSLRLSCTASGSDINDYPITWVRQAPGQGLEWIG | 146 |
| variable | FINSGGSTWYASWVKGRFTISRDDSKSIAYLQMNSLKTEDTAVYYCAR | |
| region | GYSTYYRDFNIWGQGTLVTVSS | |
| HCDR1 | GSDINDY | 10 |
| HCDR2 | NSGGS | 39 |
| HCDR3 | GYSTYYRDFNI | 99 |
| Light | DIQMTQSPSSLSASVGDRVTINCQASQSIDSNLAWFQQKPGQPPKLLIYR | 261 |
| Chain | ASNLASGVPDRFSGSGSGTDFTLTISSLEAEDVATYYCLGGVGAVSYRT | |
| | SFGGGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVYA | |
| | CEVTHQGLSSPVTKSFNRGEC | |
| Light chain | DIQMTQSPSSLSASVGDRVTINCQASQSIDSNLAWFQQKPGQPPKLLIYR | 203 |
| variable | ASNLASGVPDRFSGSGSGTDFTLTISSLEAEDVATYYCLGGVGAVSYRT | |
| region | SFGGGTKVEIK | |
| LCDR1 | QASQSIDSNLA | 140 |
| LCDR2 | RASNLAS | 142 |
| LCDR3 | LGGVGAVSYRTS | 144 |

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations such as “comprises” and “comprising”, should be understood to imply the inclusion of a stated elements or step or group of elements or steps but not the exclusion of any other element or step or group of elements or steps. The term “comprising” encompasses “including” as well as “consisting” e.g., a composition “comprising” X may consist exclusively of X or may include something additional e.g., X+Y.

The term “about” in relation to a numerical value x is optional and means, for example, x±10% or x±5%.

As used herein, the term “antibody” refers to an immunoglobulin molecule which has the ability to specifically bind to a specific antigen. An antibody often comprises a variable region and a constant region. The constant regions of antibodies may mediate the binding of the immunoglobulin to host tissues or factors, including various cells of the immune system (such as effector cells) and components of the complement system such as CIq, the first component in the classical pathway of complement activation.

A “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, VH domain comprises the following framework (FR) and CDR regions: FR1, CDR1, FR2, CDR2, FR3, CDR3, and FR4.

The assignment of amino acids to VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Chothia defined CDRs.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bi-specific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

The terms “heavy chain-only antibody”, “heavy chain antibody” and “HCAb” are used interchangeably herein and refer, in the broadest sense, to antibodies, or more or more portions of an antibody, e.g., one or more arms of an antibody, lacking the light chain of a conventional antibody. The terms specifically include, without limitation, homodimeric antibodies comprising the VH antigen-binding domain and the CH1, CH2 and CH3 constant domains; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™).

In one embodiment, a heavy chain-only antibody is composed of a variable region antigen-binding domain composed of FR1, CDR1, FR2, CDR2, FR3, CDR3, and FR4. In another embodiment, a heavy chain-only antibody is composed of an antigen-binding domain, at least part of a hinge region and CH1, CH2 and CH3 domains. In another embodiment, a heavy chain-only antibody is composed of an antigen-binding domain, at least part of a hinge region and CH2 and CH3 domains. In another embodiment, a heavy chain-only antibody is composed of an antigen-binding domain, at least part of a hinge region and a CH1 domain. In another embodiment, a heavy chain-only antibody is composed of an antigen-binding domain, at least part of a hinge region and a CH2 domain. In a further embodiment, a heavy chain-only antibody is composed of an antigen-binding domain, at least part of a hinge region and a CH3 domain. Heavy chain-only antibodies in which the CH1 and/or CH2 and/or CH3 domain is truncated are also included herein. In a further embodiment, a heavy chain is composed of an antigen binding domain, and at least one CH (CH1, CH2, CH3, or CH4) domain but no hinge region.

The heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular embodiment, a heavy chain antibody is of the IgG1, IgG2, IgG3, or IgG4 subtype, in particular the IgG1 or IgG4 subtype.

In one embodiment, the heavy-chain antibody is of the IgG1 or IgG4 subtype, wherein one or more of the CH domains is modified to alter an effector function of the antibody. Modifications of CH domains that alter effector function are further described herein.

As used herein, the term “antigen binding fragment” of an antibody refers to one or more fragments of an antibody that retain the ability to specifically bind to an antigen (e.g., ROR1). It has been shown that the antigen binding function of an antibody can be performed by fragments of a full-length antibody.

Examples of antigen binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTALIMMUNOLOGY (Paul ed., 3.sup.rd ed. 1993); (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment (Ward et al., (1989) Nature 341:544-546), which consists of a VH domain; (viii) an isolated complementarity determining region (CDR); and (ix) a nanobody, a heavy chain variable region containing a single variable domain and two constant domains. Furthermore, although the two domains of the Fv fragment, VL and VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant (epitope) and an antigen-binding site on the antibody: the lesser the value of the KD, the stronger the binding strength between an antigenic determinant (epitope) and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant (epitope) and its antigen binding site on the antibody and the number of pertinent binding sites present on the antibody. Typically, an antibody will bind with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known per se, including, for example, Scatchard analysis and/or competitive binding assays, such as radioimmunoassays (RIA), enzyme immunoassays (EIA), bio-layer interferometry (BLI) assay and sandwich competition assays, and the different variants thereof known per se in the art.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called conservative amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nat. Acad Sci. USA 81: 140-144, 1984; Kyte & Doolittle, J Mol. Biol. 157: 105-132, 1981, and Goldman et al., Ann. Rev. Biophys. Chem. 15: 321-353, 1986, all incorporated herein in their entirety by reference.

As used herein, the term “monoclonal antibody” refers to an antibody obtained from a substantially homogeneous antibody population. That is, each antibodies constituting the population are the same, except for possible naturally occurring mutations in small amount. Monoclonal antibodies are highly specific and are directed against a single antigen. The term “monoclonal antibody” herein is not limited to antibodies produced by hybridoma technology, and should not be interpreted as requiring production of antibodies by any specific method.

The term “bi-specific antibody” is in the context of the present invention to be understood as an antibody having two different antigen-binding regions defined by different antibody sequences. This can be understood as different target binding but includes as well binding to different epitopes in one target.

As used herein, the term “tumor associated antigen” refers to an antigen that is differentially expressed in cancer cells compared to normal cells, and therefore can be used to target cancer cells.

As used herein, the term “bi-specific T-cell engager” or “BiTE” refers to single polypeptide chain molecules that having two antigen-binding domains, one of which binds to a T-cell antigen and the second of which binds to an antigen present on the surface of a target (See, PCT Publication WO 05/061547; Baeuerle et al., 2008, Drugs of the Future 33: 137-147; Bargou, et al., 2008, Science 321:974-977, which are incorporated herein by reference in their entireties). Thus, the BiTE of the disclosure has an antigen binding region that binds to ROR1 and a second antigen binding region that is directed towards a T-cell antigen.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g., cancers). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

The term “Receptor tyrosine kinase like orphan receptor 1” or “ROR1” includes any ROR1 variants, isoforms and species homologs which are naturally expressed by cells of any origin, or are expressed on cells transfected with genes or cDNA encoding the ROR1 which are naturally expressed on cells of any origin.

The terms “cyno ROR1”, “cynomolgus ROR1”, and “Cynomolgus macaques ROR1” are used interchangeably herein, and are refer to cynomolgus monkey ROR1. The terms include any ROR1 variants, isoforms and species homologs which are naturally expressed by cynomolgus monkey cells, or are expressed on cells of any origin transfected with genes or cDNA encoding the cynomolgus monkey ROR1 which are naturally expressed on cynomolgus monkey cells.

The terms “human ROR1”, “huROR1” and “hROR1” are used interchangeably herein, and are refer to any ROR1 variants, isoforms and species homologs which are naturally expressed by human cells, or are expressed on cells of any origin transfected with genes or cDNA encoding the human ROR1 which are naturally expressed on human cells.

### Anti-ROR1 Antibodies

The invention provides antibodies against receptor tyrosine kinase like orphan receptor 1 (ROR1).

In a first aspect, the invention provides an antibody that specifically binds to ROR1, or an antigen binding fragment thereof, wherein the antibody comprises a heavy chain variable region (VH), and wherein the VH comprises HCDRs 1-3 of a VH having the amino acid sequence set forth in any one of SEQ ID NOs: 147-202. In some preferred embodiments, the CDRs are determined by Chothia numbering system.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 11, 40, and 100 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 41, and 101 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 42, and 102 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 40, and 103 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 43, and 104 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 13, 44, and 105 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 40, and 106 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 45, and 107 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 40, and 108 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 14, 46, and 109 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 47, and 110 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 48, and 111 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 45, and 112 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 15, 49, and 113 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 16, 50, and 114 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 51, and 115 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 18, 56, and 126 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 21, 40, and 130 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 22, 60, and 131 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 40, and 132 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 23, 61, and 133 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 19, 57, and 127 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 20, 58, and 128 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 14, 59, and 129 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 119 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 116 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 120 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 121 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 122 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 123 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 124 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 125 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 52, and 116 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 52, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 40, and 116 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 53, and 118 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 54, and 116 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 55, and 116 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 54, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 40, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 55, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 54, and 118 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 40, and 118 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 55, and 118 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 17, 55, and 103 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 55, and 117 respectively.

In some embodiments, the invention provides an antibody that specifically binds to ROR1, or an antigen binding fragment thereof, wherein the antibody comprises a heavy chain variable region (VH), wherein the VH comprises HCDRs 1-3 having the amino acid sequences set forth in SEQ ID NOs: 12, 55, and 118 respectively.

In some embodiments, the invention provides an antibody that specifically binds to ROR1, or an antigen binding fragment thereof, wherein the antibody comprises a heavy chain variable region (VH), and wherein
(1) the VH comprises an amino acid sequence having at least 80%, at least 85%, at least 90%, at least 95%, at least 98%, at least 99%, or 100% sequence identity to SEQ ID NO: 147;
(2) the VH comprises an amino acid sequence having at least 80%, at least 85%, at least 90%, at least 95%, at least 98%, at least 99%, or 100% sequence identity to SEQ ID NO: 148;
(3) the VH comprises an amino acid sequence having at least 80%, at least 85%, at least 90%, at least 95%, at least 98%, at least 99%, or 100% sequence identity to SEQ ID NO: 149;
(4) the VH comprises an amino acid sequence having at least 80%, at least 85%, at least 90%, at least 95%, at least 98%, at least 99%, or 100% sequence identity to SEQ ID NO: 150;
(5) the VH comprises an amino acid sequence having at least 80%, at least 85%, at least 90%, at least 95%, at least 98%, at least 99%, or 100% sequence identity to SEQ ID NO: 151;
(6) the VH comprises an amino acid sequence having at least 80%, at least 85%, at least 90%, at least 95%, at least 98%, at least 99%, or 100% sequence identity to SEQ ID NO: 152;
(7) the VH comprises an amino acid sequence having at least 80%, at least 85%, at least 90%, at least 95%, at least 98%, at least 99%, or 100% sequence identity to SEQ ID NO: 153;
(8) the VH comprises an amino acid sequence having at least 80%, at least 85%, at least 90%, at least 95%, at least 98%, at least 99%, or 100% sequence identity to SEQ ID NO: 154;
(9) the VH comprises an amino acid sequence having at least 80%, at least 85%, at least 90%, at least 95%, at least 98%, at least 99%, or 100% sequence identity to SEQ ID NO: 155;
(10) the VH comprises an amino acid sequence having at least 80%, at least 85%, at least 90%, at least 95%, at least 98%, at least 99%, or 100% sequence identity to SEQ ID NO: 156;
(11) the VH comprises an amino acid sequence having at least 80%, at least 85%, at least 90%, at least 95%, at least 98%, at least 99%, or 100% sequence identity to SEQ ID NO: 157;
(12) the VH comprises an amino acid sequence having at least 80%, at least 85%, at least 90%, at least 95%, at least 98%, at least 99%, or 100% sequence identity to SEQ ID NO: 158;
(13) the VH comprises an amino acid sequence having at least 80%, at least 85%, at least 90%, at least 95%, at least 98%, at least 99%, or 100% sequence identity to SEQ ID NO: 159;
(14) the VH comprises an amino acid sequence having at least 80%, at least 85%, at least 90%, at least 95%, at least 98%, at least 99%, or 100% sequence identity to SEQ ID NO: 160;
(15) the VH comprises an amino acid sequence having at least 80%, at least 85%, at least 90%, at least 95%, at least 98%, at least 99%, or 100% sequence identity to SEQ ID NO: 161;
(16) the VH comprises an amino acid sequence having at least 80%, at least 85%, at least 90%, at least 95%, at least 98%, at least 99%, or 100% sequence identity to SEQ ID NO: 162;
(17) the VH comprises an amino acid sequence having at least 80%, at least 85%, at least 90%, at least 95%, at least 98%, at least 99%, or 100% sequence identity to SEQ ID NO: 163;
(18) the VH comprises an amino acid sequence having at least 80%, at least 85%, at least 90%, at least 95%, at least 98%, at least 99%, or 100% sequence identity to SEQ ID NO: 164;
(19) the VH comprises an amino acid sequence having at least 80%, at least 85%, at least 90%, at least 95%, at least 98%, at least 99%, or 100% sequence identity to SEQ ID NO: 165;
(20) the VH comprises an amino acid sequence having at least 80%, at least 85%, at least 90%, at least 95%, at least 98%, at least 99%, or 100% sequence identity to SEQ ID NO: 166;
(21) the VH comprises an amino acid sequence having at least 80%, at least 85%, at least 90%, at least 95%, at least 98%, at least 99%, or 100% sequence identity to SEQ ID NO: 167;
(22) the VH comprises an amino acid sequence having at least 80%, at least 85%, at least 90%, at least 95%, at least 98%, at least 99%, or 100% sequence identity to SEQ ID NO: 168;
(23) the VH comprises an amino acid sequence having at least 80%, at least 85%, at least 90%, at least 95%, at least 98%, at least 99%, or 100% sequence identity to SEQ ID NO: 169;
(24) the VH comprises an amino acid sequence having at least 80%, at least 85%, at least 90%, at least 95%, at least 98%, at least 99%, or 100% sequence identity to SEQ ID NO: 170;
(25) the VH comprises an amino acid sequence having at least 80%, at least 85%, at least 90%, at least 95%, at least 98%, at least 99%, or 100% sequence identity to SEQ ID NO: 171;
(26) the VH comprises an amino acid sequence having at least 80%, at least 85%, at least 90%, at least 95%, at least 98%, at least 99%, or 100% sequence identity to SEQ ID NO: 172;
(27) the VH comprises an amino acid sequence having at least 80%, at least 85%, at least 90%, at least 95%, at least 98%, at least 99%, or 100% sequence identity to SEQ ID NO: 173;
(28) the VH comprises an amino acid sequence having at least 80%, at least 85%, at least 90%, at least 95%, at least 98%, at least 99%, or 100% sequence identity to SEQ ID NO: 174;
(29) the VH comprises an amino acid sequence having at least 80%, at least 85%, at least 90%, at least 95%, at least 98%, at least 99%, or 100% sequence identity to SEQ ID NO: 175;
(30) the VH comprises an amino acid sequence having at least 80%, at least 85%, at least 90%, at least 95%, at least 98%, at least 99%, or 100% sequence identity to SEQ ID NO: 176;
(31) the VH comprises an amino acid sequence having at least 80%, at least 85%, at least 90%, at least 95%, at least 98%, at least 99%, or 100% sequence identity to SEQ ID NO: 177;
(32) the VH comprises an amino acid sequence having at least 80%, at least 85%, at least 90%, at least 95%, at least 98%, at least 99%, or 100% sequence identity to SEQ ID NO: 178;
(33) the VH comprises an amino acid sequence having at least 80%, at least 85%, at least 90%, at least 95%, at least 98%, at least 99%, or 100% sequence identity to SEQ ID NO: 179;
(34) the VH comprises an amino acid sequence having at least 80%, at least 85%, at least 90%, at least 95%, at least 98%, at least 99%, or 100% sequence identity to SEQ ID NO: 180;
(35) the VH comprises an amino acid sequence having at least 80%, at least 85%, at least 90%, at least 95%, at least 98%, at least 99%, or 100% sequence identity to SEQ ID NO: 181;
(36) the VH comprises an amino acid sequence having at least 80%, at least 85%, at least 90%, at least 95%, at least 98%, at least 99%, or 100% sequence identity to SEQ ID NO: 182;
(37) the VH comprises an amino acid sequence having at least 80%, at least 85%, at least 90%, at least 95%, at least 98%, at least 99%, or 100% sequence identity to SEQ ID NO: 183;
(38) the VH comprises an amino acid sequence having at least 80%, at least 85%, at least 90%, at least 95%, at least 98%, at least 99%, or 100% sequence identity to SEQ ID NO: 184;
(39) the VH comprises an amino acid sequence having at least 80%, at least 85%, at least 90%, at least 95%, at least 98%, at least 99%, or 100% sequence identity to SEQ ID NO: 185;
(40) the VH comprises an amino acid sequence having at least 80%, at least 85%, at least 90%, at least 95%, at least 98%, at least 99%, or 100% sequence identity to SEQ ID NO: 186;
(41) the VH comprises an amino acid sequence having at least 80%, at least 85%, at least 90%, at least 95%, at least 98%, at least 99%, or 100% sequence identity to SEQ ID NO: 187;
(42) the VH comprises an amino acid sequence having at least 80%, at least 85%, at least 90%, at least 95%, at least 98%, at least 99%, or 100% sequence identity to SEQ ID NO: 188;
(43) the VH comprises an amino acid sequence having at least 80%, at least 85%, at least 90%, at least 95%, at least 98%, at least 99%, or 100% sequence identity to SEQ ID NO: 189;
(44) the VH comprises an amino acid sequence having at least 80%, at least 85%, at least 90%, at least 95%, at least 98%, at least 99%, or 100% sequence identity to SEQ ID NO: 190;
(45) the VH comprises an amino acid sequence having at least 80%, at least 85%, at least 90%, at least 95%, at least 98%, at least 99%, or 100% sequence identity to SEQ ID NO: 191;
(46) the VH comprises an amino acid sequence having at least 80%, at least 85%, at least 90%, at least 95%, at least 98%, at least 99%, or 100% sequence identity to SEQ ID NO: 192;
(47) the VH comprises an amino acid sequence having at least 80%, at least 85%, at least 90%, at least 95%, at least 98%, at least 99%, or 100% sequence identity to SEQ ID NO: 193;
(48) the VH comprises an amino acid sequence having at least 80%, at least 85%, at least 90%, at least 95%, at least 98%, at least 99%, or 100% sequence identity to SEQ ID NO: 194;
(49) the VH comprises an amino acid sequence having at least 80%, at least 85%, at least 90%, at least 95%, at least 98%, at least 99%, or 100% sequence identity to SEQ ID NO: 195;
(50) the VH comprises an amino acid sequence having at least 80%, at least 85%, at least 90%, at least 95%, at least 98%, at least 99%, or 100% sequence identity to SEQ ID NO: 196;
(51) the VH comprises an amino acid sequence having at least 80%, at least 85%, at least 90%, at least 95%, at least 98%, at least 99%, or 100% sequence identity to SEQ ID NO: 197;
(52) the VH comprises an amino acid sequence having at least 80%, at least 85%, at least 90%, at least 95%, at least 98%, at least 99%, or 100% sequence identity to SEQ ID NO: 198;
(53) the VH comprises an amino acid sequence having at least 80%, at least 85%, at least 90%, at least 95%, at least 98%, at least 99%, or 100% sequence identity to SEQ ID NO: 199;
(54) the VH comprises an amino acid sequence having at least 80%, at least 85%, at least 90%, at least 95%, at least 98%, at least 99%, or 100% sequence identity to SEQ ID NO: 200;
(55) the VH comprises an amino acid sequence having at least 80%, at least 85%, at least 90%, at least 95%, at least 98%, at least 99%, or 100% sequence identity to SEQ ID NO: 201; or
(56) the VH comprises an amino acid sequence having at least 80%, at least 85%, at least 90%, at least 95%, at least 98%, at least 99%, or 100% sequence identity to SEQ ID NO: 202.

In some embodiments, the antibody comprises an Fe region. In some embodiments, the Fe region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fe region is human IgG1 Fc.

In one embodiment, the Fc region is effector-function-deficient. For example, the Fe region may be of an IgG1 isotype, or a non-IgG1 type, e.g., IgG2, IgG3 or IgG4, which has been mutated such that the ability to mediate effector functions, such as ADCC, has been reduced or even eliminated. Such mutations have e.g., been described in Dall'Acqua W F et al., J Immunol. 177(2):1129-1138 (2006) and Hezareh M, J Virol.; 75(24):12161-12168 (2001). In some embodiments, the Fe region of the antibody comprises a wild type IgG1 Fe with L234A, L235A and G237A mutations.

In some embodiments, the antibody is mutated at one or more post-translational modifications sites. In one embodiment, the Fe region comprises a mutation removing the acceptor site for Asn-linked glycosylation or is manipulated to eliminate the effector function of the antibody.

In some embodiments, the invention provides an antibody that specifically binds to ROR1, or an antigen binding fragment thereof, wherein the antibody comprises a heavy chain (HC), and wherein
(1) the HC comprises an amino acid sequence having at least 80%, at least 85%, at least 90%, at least 95%, at least 98%, at least 99%, or 100% sequence identity to SEQ ID NO: 205;
(2) the HC comprises an amino acid sequence having at least 80%, at least 85%, at least 90%, at least 95%, at least 98%, at least 99%, or 100% sequence identity to SEQ ID NO: 206;
(3) the HC comprises an amino acid sequence having at least 80%, at least 85%, at least 90%, at least 95%, at least 98%, at least 99%, or 100% sequence identity to SEQ ID NO: 207;
(4) the HC comprises an amino acid sequence having at least 80%, at least 85%, at least 90%, at least 95%, at least 98%, at least 99%, or 100% sequence identity to SEQ ID NO: 208;
(5) the HC comprises an amino acid sequence having at least 80%, at least 85%, at least 90%, at least 95%, at least 98%, at least 99%, or 100% sequence identity to SEQ ID NO: 209;
(6) the HC comprises an amino acid sequence having at least 80%, at least 85%, at least 90%, at least 95%, at least 98%, at least 99%, or 100% sequence identity to SEQ ID NO: 210;
(7) the HC comprises an amino acid sequence having at least 80%, at least 85%, at least 90%, at least 95%, at least 98%, at least 99%, or 100% sequence identity to SEQ ID NO: 211;
(8) the HC comprises an amino acid sequence having at least 80%, at least 85%, at least 90%, at least 95%, at least 98%, at least 99%, or 100% sequence identity to SEQ ID NO: 212;
(9) the HC comprises an amino acid sequence having at least 80%, at least 85%, at least 90%, at least 95%, at least 98%, at least 99%, or 100% sequence identity to SEQ ID NO: 213;
(10) the HC comprises an amino acid sequence having at least 80%, at least 85%, at least 90%, at least 95%, at least 98%, at least 99%, or 100% sequence identity to SEQ ID NO: 214;
(11) the HC comprises an amino acid sequence having at least 80%, at least 85%, at least 90%, at least 95%, at least 98%, at least 99%, or 100% sequence identity to SEQ ID NO: 215;
(12) the HC comprises an amino acid sequence having at least 80%, at least 85%, at least 90%, at least 95%, at least 98%, at least 99%, or 100% sequence identity to SEQ ID NO: 216;
(13) the HC comprises an amino acid sequence having at least 80%, at least 85%, at least 90%, at least 95%, at least 98%, at least 99%, or 100% sequence identity to SEQ ID NO: 217;
(14) the HC comprises an amino acid sequence having at least 80%, at least 85%, at least 90%, at least 95%, at least 98%, at least 99%, or 100% sequence identity to SEQ ID NO: 218;
(15) the HC comprises an amino acid sequence having at least 80%, at least 85%, at least 90%, at least 95%, at least 98%, at least 99%, or 100% sequence identity to SEQ ID NO: 219;
(16) the HC comprises an amino acid sequence having at least 80%, at least 85%, at least 90%, at least 95%, at least 98%, at least 99%, or 100% sequence identity to SEQ ID NO: 220;
(17) the HC comprises an amino acid sequence having at least 80%, at least 85%, at least 90%, at least 95%, at least 98%, at least 99%, or 100% sequence identity to SEQ ID NO: 221;
(18) the HC comprises an amino acid sequence having at least 80%, at least 85%, at least 90%, at least 95%, at least 98%, at least 99%, or 100% sequence identity to SEQ ID NO: 222;
(19) the HC comprises an amino acid sequence having at least 80%, at least 85%, at least 90%, at least 95%, at least 98%, at least 99%, or 100% sequence identity to SEQ ID NO: 223;
(20) the HC comprises an amino acid sequence having at least 80%, at least 85%, at least 90%, at least 95%, at least 98%, at least 99%, or 100% sequence identity to SEQ ID NO: 224;
(21) the HC comprises an amino acid sequence having at least 80%, at least 85%, at least 90%, at least 95%, at least 98%, at least 99%, or 100% sequence identity to SEQ ID NO: 225;
(22) the HC comprises an amino acid sequence having at least 80%, at least 85%, at least 90%, at least 95%, at least 98%, at least 99%, or 100% sequence identity to SEQ ID NO: 226;
(23) the HC comprises an amino acid sequence having at least 80%, at least 85%, at least 90%, at least 95%, at least 98%, at least 99%, or 100% sequence identity to SEQ ID NO: 227;
(24) the HC comprises an amino acid sequence having at least 80%, at least 85%, at least 90%, at least 95%, at least 98%, at least 99%, or 100% sequence identity to SEQ ID NO: 228;
(25) the HC comprises an amino acid sequence having at least 80%, at least 85%, at least 90%, at least 95%, at least 98%, at least 99%, or 100% sequence identity to SEQ ID NO: 229;
(26) the HC comprises an amino acid sequence having at least 80%, at least 85%, at least 90%, at least 95%, at least 98%, at least 99%, or 100% sequence identity to SEQ ID NO: 230;
(27) the HC comprises an amino acid sequence having at least 80%, at least 85%, at least 90%, at least 95%, at least 98%, at least 99%, or 100% sequence identity to SEQ ID NO: 231;
(28) the HC comprises an amino acid sequence having at least 80%, at least 85%, at least 90%, at least 95%, at least 98%, at least 99%, or 100% sequence identity to SEQ ID NO: 232;
(29) the HC comprises an amino acid sequence having at least 80%, at least 85%, at least 90%, at least 95%, at least 98%, at least 99%, or 100% sequence identity to SEQ ID NO: 233;
(30) the HC comprises an amino acid sequence having at least 80%, at least 85%, at least 90%, at least 95%, at least 98%, at least 99%, or 100% sequence identity to SEQ ID NO: 234;
(31) the HC comprises an amino acid sequence having at least 80%, at least 85%, at least 90%, at least 95%, at least 98%, at least 99%, or 100% sequence identity to SEQ ID NO: 235;
(32) the HC comprises an amino acid sequence having at least 80%, at least 85%, at least 90%, at least 95%, at least 98%, at least 99%, or 100% sequence identity to SEQ ID NO: 236;
(33) the HC comprises an amino acid sequence having at least 80%, at least 85%, at least 90%, at least 95%, at least 98%, at least 99%, or 100% sequence identity to SEQ ID NO: 237;
(34) the HC comprises an amino acid sequence having at least 80%, at least 85%, at least 90%, at least 95%, at least 98%, at least 99%, or 100% sequence identity to SEQ ID NO: 238;
(35) the HC comprises an amino acid sequence having at least 80%, at least 85%, at least 90%, at least 95%, at least 98%, at least 99%, or 100% sequence identity to SEQ ID NO: 239;
(36) the HC comprises an amino acid sequence having at least 80%, at least 85%, at least 90%, at least 95%, at least 98%, at least 99%, or 100% sequence identity to SEQ ID NO: 240;
(37) the HC comprises an amino acid sequence having at least 80%, at least 85%, at least 90%, at least 95%, at least 98%, at least 99%, or 100% sequence identity to SEQ ID NO: 241;
(38) the HC comprises an amino acid sequence having at least 80%, at least 85%, at least 90%, at least 95%, at least 98%, at least 99%, or 100% sequence identity to SEQ ID NO: 242;
(39) the HC comprises an amino acid sequence having at least 80%, at least 85%, at least 90%, at least 95%, at least 98%, at least 99%, or 100% sequence identity to SEQ ID NO: 243;
(40) the HC comprises an amino acid sequence having at least 80%, at least 85%, at least 90%, at least 95%, at least 98%, at least 99%, or 100% sequence identity to SEQ ID NO: 244;
(41) the HC comprises an amino acid sequence having at least 80%, at least 85%, at least 90%, at least 95%, at least 98%, at least 99%, or 100% sequence identity to SEQ ID NO: 245;
(42) the HC comprises an amino acid sequence having at least 80%, at least 85%, at least 90%, at least 95%, at least 98%, at least 99%, or 100% sequence identity to SEQ ID NO: 246;
(43) the HC comprises an amino acid sequence having at least 80%, at least 85%, at least 90%, at least 95%, at least 98%, at least 99%, or 100% sequence identity to SEQ ID NO: 247;
(44) the HC comprises an amino acid sequence having at least 80%, at least 85%, at least 90%, at least 95%, at least 98%, at least 99%, or 100% sequence identity to SEQ ID NO: 248;
(45) the HC comprises an amino acid sequence having at least 80%, at least 85%, at least 90%, at least 95%, at least 98%, at least 99%, or 100% sequence identity to SEQ ID NO: 249;
(46) the HC comprises an amino acid sequence having at least 80%, at least 85%, at least 90%, at least 95%, at least 98%, at least 99%, or 100% sequence identity to SEQ ID NO: 250;
(47) the HC comprises an amino acid sequence having at least 80%, at least 85%, at least 90%, at least 95%, at least 98%, at least 99%, or 100% sequence identity to SEQ ID NO: 251;
(48) the HC comprises an amino acid sequence having at least 80%, at least 85%, at least 90%, at least 95%, at least 98%, at least 99%, or 100% sequence identity to SEQ ID NO: 252;
(49) the HC comprises an amino acid sequence having at least 80%, at least 85%, at least 90%, at least 95%, at least 98%, at least 99%, or 100% sequence identity to SEQ ID NO: 253;
(50) the HC comprises an amino acid sequence having at least 80%, at least 85%, at least 90%, at least 95%, at least 98%, at least 99%, or 100% sequence identity to SEQ ID NO: 254;
(51) the HC comprises an amino acid sequence having at least 80%, at least 85%, at least 90%, at least 95%, at least 98%, at least 99%, or 100% sequence identity to SEQ ID NO: 255;
(52) the HC comprises an amino acid sequence having at least 80%, at least 85%, at least 90%, at least 95%, at least 98%, at least 99%, or 100% sequence identity to SEQ ID NO: 256;
(53) the HC comprises an amino acid sequence having at least 80%, at least 85%, at least 90%, at least 95%, at least 98%, at least 99%, or 100% sequence identity to SEQ ID NO: 257;
(54) the HC comprises an amino acid sequence having at least 80%, at least 85%, at least 90%, at least 95%, at least 98%, at least 99%, or 100% sequence identity to SEQ ID NO: 258;
(55) the HC comprises an amino acid sequence having at least 80%, at least 85%, at least 90%, at least 95%, at least 98%, at least 99%, or 100% sequence identity to SEQ ID NO: 259; or
(56) the HC comprises an amino acid sequence having at least 80%, at least 85%, at least 90%, at least 95%, at least 98%, at least 99%, or 100% sequence identity to SEQ ID NO: 260.

In some embodiments, the antibody of the invention comprises a heavy chain and a light chain. In some embodiments, the antibody of the invention comprises two heavy chains and two light chains.

Based on the amino acid sequence of heavy chain constant regions of the antibody, an immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (λ) chain or a kappa (κ) chain, based on the amino acid sequence of the light chain. The antibodies disclosed herein can be of any classes or subtypes above.

In some embodiments, the antibody can be of an isotype selected from the group consisting of IgG, IgA, IgM, IgE and IgD. In some embodiments, the antibody can be of a subtype selected from the group consisting of IgG1, IgG2, IgG3, and IgG4. In a preferred embodiment, the antibody is an IgG1 antibody.

In some embodiments, the antibody of the invention does not comprise a light chain. In some specific embodiments, the antibody only comprises one or two heavy chains. In some preferred embodiments, the antibody of the invention is composed of one heavy chain. In some preferred embodiments, the antibody of the invention is composed of two heavy chains.

In some embodiments, the antibody of the invention is a homodimeric antibody comprising the VH antigen-binding domain and the CH2 and CH3 constant domains.

In one embodiment, the antibody of the invention is a heavy chain-only antibody composed of a variable region antigen-binding domain composed of framework 1, CDR1, framework 2, CDR2, framework 3, CDR3, and framework 4.

In another embodiment, the antibody of the invention is a heavy chain-only antibody composed of an antigen-binding domain, at least part of a hinge region and CH1, CH2 and CH3 domains. In another embodiment, a heavy chain-only antibody is composed of an antigen-binding domain, at least part of a hinge region and CH2 and CH3 domains. In another embodiment, the antibody of the invention is a heavy chain-only antibody composed of an antigen-binding domain, at least part of a hinge region and a CH1 domain. In another embodiment, the antibody of the invention is a heavy chain-only antibody composed of an antigen-binding domain, at least part of a hinge region and a CH2 domain. In a further embodiment, the antibody of the invention is a heavy chain-only antibody composed of an antigen-binding domain, at least part of a hinge region and a CH3 domain. In a further embodiment, the antibody of the invention is a heavy chain is composed of an antigen binding domain, and at least one CH (CH1, CH2, CH3, or CH4) domain but no hinge region.

In some embodiments, the antibody of the invention is a heavy chain-only antibody in the form of a dimer, in which two heavy chains are disulfide bonded or otherwise, covalently or non-covalently, attached with each other.

In some embodiments, the antibody of the invention is a heavy chain-only antibody which belongs to the IgG, IgM, IgA, IgD or IgE subclass antibodies.

In some embodiments, the antibody of the invention is a heavy chain-only antibody of the IgG1, IgG2, IgG3, or IgG4 subtype, in particular the IgG1 or IgG4 subtype.

In one embodiment, the antibody of the invention is a heavy-chain antibody of the IgG1 or IgG4 subtype, wherein one or more of the CH domains is modified to alter an effector function of the antibody.

The antibody disclosed herein can be an intact antibody or the antigen binding fragment thereof. The antigen binding fragment can be any fragments of the antibody that retain the ability to specifically bind to ROR1. Examples of antigen binding fragments include but are not limited to a Fab fragment; a F(ab′)2 fragment; a Fab′ fragment; a Fd fragment; a Fd′ fragment; a dAb fragment; an isolated complementarity determining region (CDR); a nanobody; a linear antibody comprising a pair of tandem Fd segments (VH-CH1-VH-CH1), and a modified version of any of the foregoing fragments, which retains antigen binding activity.

In some embodiments, the antigen binding fragment can be selected from the group consisting of HCAb, VHH, nanobody, Fab, Fab′, F(ab′)₂, Fd, Fd′, and dAb.

In some embodiments, the antibody of the invention is a chimeric antibody, a humanized antibody, or a human antibody.

In some embodiments, the antibody of the invention is a monoclonal antibody, a bi-specific or a multi-specific antibody.

In some embodiments, the antibody is monovalent, bivalent or multivalent.

In some embodiments, the antibody or antigen binding fragment of the invention is attached to a fluorescent label, radiolabel or cytotoxic agent.

### Bi-Specific Antibody

In a second aspect, the present application provides a bi-specific or a multi-specific antibody. In some embodiments, the antibody is a bi-specific antibody which further comprises a second antigen binding region binding to a second antigen. In some embodiments, the second antigen can be a tumor associated antigen, an immune checkpoint molecule or an immune cell antigen.

Many tumor associated antigens associated with specific cancers have been identified in the art. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

Examples of tumor associated antigens include but are not limited to 5T4, alphafetoprotein, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFR, EGFRvIII, epithelial tumor antigen, ERBB2, FLT3, folate binding protein, GD2, GD3, HIV-1 envelope glycoprotein gp41, HIV-1 envelope glycoprotein gp120, melanoma-associated antigen, ROR1, MUC-1, mutated p53, mutated ras, ROR1, VEGFR2, and combinations thereof.

In some embodiments, the second antigen is an immune cell antigen. In some embodiments, the T-cell antigen can be selected from the group consisting of T cell receptor (TCR), CD3, CD4, CD8, CD16, CD25, CD28, CD44, CD62L, CD69, ICOS, 41-BB (CD137), and NKG2D or any combination thereof. Preferably, the second antigen is CD3.

In some embodiments, the second antigen is an immune checkpoint molecule. In some embodiments, the immune checkpoint molecule can be selected from the group consisting of PD-1, PD-L1, CTLA-4, and the like.

In some embodiments, the bi-specific antibody comprises a single polypeptide chain comprising the first antigen binding region and the second antigen binding region, and optionally an Fc region. The Fc region may be of any isotype, including, but not limited to, IgG1, IgG2, IgG3 and IgG4, and may comprise one or more mutations or modifications. In one embodiment, the Fe region is of IgG1 isotype or derived therefrom, optionally with one or more mutations or modifications.

In one embodiment, the Fc region is effector-function-deficient. For example, the Fc region may be of an IgG1 isotype, or a non-IgG1 type, e.g. IgG2, IgG3 or IgG4, which has been mutated such that the ability to mediate effector functions, such as ADCC, has been reduced or even eliminated. Such mutations have e.g. been described in Dall'Acqua W F et al., J Immunol. 177(2):1129-1138 (2006) and Hezareh M, J Virol.; 75(24):12161-12168 (2001).

In one embodiment, the Fe region comprises a mutation removing the acceptor site for Asn-linked glycosylation or is otherwise manipulated to change the glycosylation properties. For example, in an IgG1 Fe region, an N297Q mutation can be used to remove an Asn-linked glycosylation site. Accordingly, in a specific embodiment, Fe region comprise an IgG1 wildtype sequence with an N297Q mutation. For example, in an IgG1 Fe region, an N297Q mutation can be used to remove an Asn-linked glycosylation site. Accordingly, in a specific embodiment, Fe region comprise an IgG1 wildtype sequence with an N297Q mutation.

In a further embodiment, the Fe region is glyco-engineered to reduce fucose and thus enhance ADCC, e.g. by addition of compounds to the culture media during antibody production as described in US2009317869 or as described in van Berkel et al. (2010) Biotechnol. Bioeng. 105:350 or by using FUT8 knockout cells, e.g. as described in Yamane-Ohnuki et al. (2004) Biotechnol. Bioeng 87:614. ADCC may alternatively be optimized using the method described by Umana et al. (1999) Nature Biotech 17:176. In a further embodiment, the Fc region has been engineered to enhance complement activation, e.g. as described in Natsume et al. (2009) Cancer Sci. 100:2411.

### Nucleic Acids

In a third aspect, the invention provides a nucleic acid comprising a nucleotide sequence encoding the anti-ROR1 antibody or the antigen binding fragment thereof disclosed herein or the bi-specific antibody or the antigen binding fragment thereof disclosed herein.

The term “polynucleotide” or “nucleic acid” includes both single-stranded and double-stranded nucleotide polymers. The nucleotides comprising the nucleic acid can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

For example, the invention provides nucleic acid molecules encoding any one of the heavy chain variable region sequences disclosed herein. The invention also provides nucleic acid molecules that are at least 90%, at least 95%, at least 98% or at least 99% identical to nucleic acids encoding any one of the heavy chain variable region sequences disclosed herein.

For example, the invention provides nucleic acid molecules encoding any one of the light chain variable region sequences disclosed herein. The invention also provides nucleic acid molecules that are at least 90%, at least 95%, at least 98% or at least 99% identical to nucleic acids encoding any one of the light chain variable region sequences disclosed herein.

For example, the invention provides nucleic acid molecules encoding: (i) any one of the heavy chain variable region sequences disclosed herein and (ii) any one of the light chain variable region sequences disclosed herein. The invention also provides nucleic acid molecules that are at least 90%, at least 95%, at least 98% or at least 99% identical to nucleic acids encoding: (i) any one of the heavy chain variable region sequences disclosed herein and (ii) any one of the light chain variable region sequences disclosed herein.

In some embodiments, the nucleic acid is ribonucleic acid (RNA) or deoxyribonucleic acid (DNA). In some embodiments, the invention provides a ribonucleic acid (RNA) comprising a nucleotide sequence encoding the anti-ROR1 antibody or the antigen binding fragment thereof disclosed herein or the bi-specific antibody or the antigen binding fragment thereof disclosed herein. In some embodiments, the invention provides a deoxyribonucleic acid (DNA) comprising a deoxynucleotide sequence encoding the anti-ROR1 antibody or the antigen binding fragment thereof disclosed herein or the bi-specific antibody or the antigen binding fragment thereof disclosed herein.

Accordingly, the deoxyribonucleic acid (DNA) comprising a deoxynucleotide sequence encoding the anti-ROR1 antibody or the antigen binding fragment thereof disclosed herein or the bi-specific antibody or the antigen binding fragment thereof disclosed herein is used for treating a disease. In some embodiments, the disease is a cancer, preferably is a ROR1 positive cancer. In some embodiments, the cancer is selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

Accordingly, the ribonucleic acid (RNA) comprising a deoxynucleotide sequence encoding the anti-ROR1 antibody or the antigen binding fragment thereof disclosed herein or the bi-specific antibody or the antigen binding fragment thereof disclosed herein may be used for treating a disease. In some embodiments, the disease is a cancer. In some embodiments, the cancer is selected from the group consisting of a ROR1 positive cancer, preferably selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

In some embodiments, the deoxyribonucleic acid (DNA) or ribonucleic acid (RNA) may be introduced into the cells of a human body in vivo. In some embodiments, the deoxyribonucleic acid (DNA) or ribonucleic acid (RNA) of the invention is comprised in a vector or a delivering agent. In some embodiments, the deoxyribonucleic acid (DNA) of the invention is integrated into the genome of a cell.

### Vectors

In the fourth aspect, the invention further provides a vector, which comprises the nucleic acid comprising a nucleotide sequence encoding the anti-ROR1 antibody or the antigen binding fragment thereof disclosed herein or the bi-specific antibody or the antigen binding fragment thereof disclosed herein.

In some embodiments, the vector is a recombinant expression vector capable of expressing a polypeptide comprising a heavy or light chain variable region of an anti-ROR1 antibody. For example, the invention provides recombinant expression vectors comprising any of the nucleic acid molecules mentioned above.

Any vector may be suitable for the present disclosure. In some embodiments, the vector is a viral vector. In some embodiments, the vector is a retroviral vector, a DNA vector, a murine leukemia virus vector, an SFG vector, a plasmid, an RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also may be used. Examples of plant expression vectors useful in the context of the disclosure comprise pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, may be prepared to contain a replication system functional in a prokaryotic or eukaryotic host cell. Replication systems may be derived, e.g., from ColEl, 2μ plasmid, λ, SV40, bovine papilloma virus, and the like.

Accordingly, the vector may be used for treating a disease. In some embodiments, the disease is a cancer. In some embodiments, the cancer is selected from the group consisting of a ROR1 positive cancer, preferably selected from the group consisting of B-cell leukemia, lymphoma, acute mycloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer. The vector of the invention may be introduced into a cell. In some embodiments, the vector of the invention may be introduced into a cell in vitro or ex vivo. Optionally, the cell introduced with the vector may subsequently be administered into the body of a subject. In some embodiments, the vector of the invention may be introduced into a cell in vivo.

For example, the vector may be an adenoviral vector comprising a nucleotide sequence encoding the anti-ROR1 antibody or the antigen binding fragment thereof disclosed herein or the bi-specific antibody or the antigen binding fragment thereof disclosed herein. The vector may be administered into the body of a subject, and then enter into a cell of the subject in vivo, thereby the nucleotide sequence encoding the anti-ROR1 antibody or the antigen binding fragment thereof disclosed herein or the bi-specific antibody or the antigen binding fragment thereof disclosed herein is integrated into the genome of the cell, and subsequently the cell expresses the anti-ROR1 antibody or the antigen binding fragment thereof disclosed herein so as to treat the diseases disclosed herein.

### Host Cells

In the fifth aspect, the invention further provides a host cell comprising the nucleic acid disclosed herein or the vector disclosed herein.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, host cells include, for example, CHO cells, such as CHOS cells and CHO-K1 cells, or HEK293 cells, such as HEK293A, HEK293T and HEK293FS.

The host cell of the invention is prepared by introducing the vector disclosed herein or the nucleic acid disclosed herein in vitro or ex vivo. The host cell of the invention may be administered into the body of a subject, and the host cell expresses the anti-ROR1 antibody or the antigen binding fragment thereof disclosed herein in vivo so as to treat the diseases disclosed herein.

The invention further provides host cells into which any of the vectors mentioned above have been introduced. The invention further provides methods of producing the antibodies and antibody fragments of the invention by culturing the host cells under conditions permitting production of the antibodies or antibody fragments and recovering the antibodies and antibody fragments so produced.

### Antibody-Drug Conjugate

In the sixth aspect, the invention provides an antibody-drug conjugate (ADC), comprising the antibody or the antigen-binding fragment thereof of the first aspect of the invention or the bi-specific antibody of the second aspect of the invention.

In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate is often referred to as an “immunoconjugate.” When the conjugate comprises an antibody linked to a drug (e.g., a cytotoxic agent), the conjugate is often referred to as an “antibody-drug conjugate” or “ADC.” Other antibody conjugates include, for example, multi-specific (such as bi-specific or trispecific) antibodies.

In some embodiments, the effector molecule can be a detectable label or an immunotoxin. Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain 1a of PE and the B chain of DT) and replacing it with a different targeting moiety, such as an antibody. The term “conjugated” or “linked” may refer to making two polypeptides into one contiguous polypeptide molecule. In one embodiment, an antibody is joined to an effector molecule. In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule.

The invention provides immunoconjugates that include a monoclonal antibody or antigen-binding fragment disclosed herein and an effector molecule. In some embodiments, the effector molecule is a toxin, such as, but not limited to, Pseudomonas exotoxin or a variant thereof. In other embodiments, the effector molecule is a detectable label, such as, but not limited to, a fluorophore, an enzyme or a radioisotope.

The disclosed monoclonal antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a tumor cell). Conversely, where it is desired to invoke a non-lethal biological response, the therapeutic agent can be conjugated to a non-lethal pharmacological agent or a liposome containing a non-lethal pharmacological agent.

With the therapeutic agents and antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to an antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site.

Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies one skilled in the art will be able to determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bi-specific antibody or a diabody), a detection agent, a pharmaceutical agent, and/or a protein or peptide that can mediate association of the antibody or antibody portion with another molecule (such as a strep tavidin core region or a polyhistidine tag).

One type of derivatized antibody is produced by cross-linking two or more antibodies (of the same type or of different types, such as to create bi-specific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

The antibody can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

An antibody may be fused to a self-labelling protein tag (e.g. HaloTag). For example, the protein tag could be cloned at the end of a constant region. HaloTag is a self-labelling protein tag derived from a bacterial enzyme (a haloalkane dehalogenase), designed to covalently bind to a synthetic ligand. In some instances, the synthetic ligand comprises a chloroalkane linker attached to a fluorophore, such as a near-infrared fluorophore (Los et al. (2008) ACS Chem Biol. 3(6):373-82).

An antibody may be labeled with a magnetic agent, such as gadolinium. Antibodies can also be labeled with lanthanides (such as europium and dysprosium), and manganese.

Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one embodiment, the toxin is Pseudomonas exotoxin (PE) (U.S. Pat. No. 5,602,095).

The antibodies described herein can also be used to target any number of different diagnostic or therapeutic compounds to cells expressing the tumor or viral antigen on their surface. Thus, an antibody of the present disclosure can be attached directly or via a linker to a drug that is to be delivered directly to cells expressing cell-surface antigen. This can be done for therapeutic, diagnostic or research purposes. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The ADCs disclosed herein can be used for the treatment of a cancer alone or in combination with another therapeutic agent and/or in combination with any standard therapy for the treatment of cancer (such as surgical resection of the tumor, chemotherapy or radiation therapy), wherein the cancer is responsive to decreasing, inhibiting and/or blocking immune regulatory function or activity mediated by ROR1.

### Pharmaceutical Compositions

In the seventh aspect, the invention provides a pharmaceutical composition comprising (i) the antibody or the antigen binding fragment thereof of the first aspect of the invention, or the bi-specific antibody of the second aspect of the invention, or the nucleic acid of the third aspect of the invention, or the vector of the fourth aspect of the invention, or the host cell of the fifth aspect of the invention, or the ADC of the sixth aspect of the invention; and optionally (ii) a pharmaceutically acceptable carrier or excipient.

The invention provides pharmaceutical composition comprising an antibody of the invention. In some embodiments, the pharmaceutical composition further comprises a pharmaceutically acceptable carrier. The term “pharmaceutically acceptable carrier” includes any and all solvents, buffers, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion). For example, in some embodiments, a composition for intravenous administration typically is a solution in sterile isotonic aqueous buffer.

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention is dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention provides therapeutic compositions comprising the anti-ROR1 antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

### Methods of Production

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103) Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “humanized antibodies”, “human antibodies”, or “fully human antibodies” herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, humanized antibodies can be produced in transgenic plants, as an inexpensive production alternative to existing mammalian systems. For example, the transgenic plant may be a tobacco plant, i.e., Nicotiana benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

In addition, an (human) antibody of interest may be produced in fungi. For example, the fungus may be Myceliophthora thermophila (e.g. Myceliophthora thermophila strain C1; Visser et al. (2011) Industrial Biotechnology 7(3):214-223). Other examples include Aspergillus species (e.g. A. oryzae (Huynh et al. (2020) Fungal Biology and Biotechnology 7:7), A. niger (Ward et al. (2004) Environ. Microbiol. 70:2567-76), or A. awamori (Joosten et al. (2003) Microb. Cell Fact 2:1)) and Trichoderma species (e.g. T. reesei (Nyyssönen et al. (1993) Biotechnology 11:591-595)). In other instances, the fungus may be a yeast, such as Saccharomyces cerevisiae, Candida boidinii, Hansenula polymorpha, Pichia methanolica, Pichia pastoris, Yarrowia lipolytica, Kluyveromyces lactis or Ogataea minuta (Joosten et al. (2003); Suzuki et al. (2017) J Biosci Bioeng. 124:156-63).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in WO 2006/008548, WO 2007/096779, WO 2010/109165, WO 2010/070263, WO 2014/141189 and WO 2014/141192.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include Moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell cytoplasm. Avipox virus vectors result in only a short-term expression of the nucleic acid. Adenovirus vectors, adeno-associated virus vectors and herpes simplex virus (HSV) vectors are preferred for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of ROR1 in a sample. The antibody can also be used to try to bind to ROR1.

Methods for the screening of antibodies that possess the desired specificity include, but are not limited to, enzyme linked immunosorbent assay (ELISA) and other immunologically mediated techniques known within the art.

### Therapeutic Methods

The antibodies provide herein can be administered to slow or inhibit the progression of a ROR1-positive cancer, and/or inhibit the metastasis of a ROR1-positive cancer. In these applications, a therapeutically effective amount of a composition is administered to a subject in an amount sufficient to inhibit growth, replication or metastasis of cancer cells, or to inhibit a sign or a symptom of the cancer. Suitable subjects may include those diagnosed with a cancer that expresses ROR1, such as B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

Administration of an antibody disclosed herein can also be accompanied by administration of other anti-cancer agents or therapeutic treatments (such as surgical resection of a tumor). Any suitable anti-cancer agent can be administered in combination with the antibodies disclosed herein. Exemplary anti-cancer agents include, but are not limited to, chemotherapeutic agents, such as, for example, mitotic inhibitors, alkylating agents, antimetabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

In some embodiments, the antibody or the antigen binding fragment thereof, the bi-specific antibody, the nucleic acid, the vector, the host cell, the antibody-drug conjugate, or the pharmaceutical composition of the invention may be administered in combination with an antibody, a chemotherapeutic agent, a siRNA, an antisense oligonucleotide, a polypeptide, and a small molecule drug.

Another common treatment for some types of cancer is surgical treatment, for example surgical resection of a metastatic tumor. Another example of a treatment is radiotherapy, for example administration of radioactive material or energy (such as external beam therapy) to the tumor site to help eradicate the tumor or shrink it prior to surgical resection.

### Methods for Diagnosis and Detection

In an aspect, the invention provides a method for determining a subject suffering from a cancer or having a risk of developing a ROR1 positive cancer, wherein the method comprises:
(a) obtaining a biological sample from the subject,
(b) contacting the sample with the antibody or the antigen binding fragment thereof of the invention, and
(c) detecting binding of the antibody to the sample,
wherein an increase in binding of the antibody or antigen binding fragment thereof to the sample as compared to binding of the antibody or antigen binging fragment thereof to a control sample identifies the subject as having a ROR1 positive cancer.

In another aspect, the invention provides a method for imaging a ROR1 positive cancer in a subject, wherein the method comprises:
(a) administering the antibody or antigen binding fragment thereof of the invention to the subject, wherein the antibody is conjugated to a detectable marker, and
(b) detecting the presence of the marker

Methods are provided herein for detecting ROR1 protein in vitro or in vivo. In some cases, ROR1 expression is detected in a biological sample. The sample can be any sample, including, but not limited to, tumor samples, blood samples, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

Provided herein is a method of determining if a subject has a cancer by contacting a sample from the subject with a ROR1-specific monoclonal antibody disclosed herein; and detecting binding of the antibody to the sample. An increase in binding of the antibody to the sample as compared to binding of the antibody to a control sample identifies the subject as having a cancer.

In another embodiment, provided is a method of diagnosing of a cancer in a subject by contacting a sample from a subject diagnosed with a cancer with a ROR1-specific monoclonal antibody disclosed herein; and detecting binding of the antibody to the sample. An increase in binding of the antibody to the sample as compared to binding of the antibody to a control sample confirms the diagnosis of a cancer in the subject.

In some examples of the disclosed methods, the monoclonal antibody is directly labeled.

In other examples, the methods further include contacting a second antibody that specifically binds the monoclonal antibody with the sample; and detecting the binding of the second antibody. An increase in binding of the second antibody to the sample as compared to binding of the second antibody to a control sample detects a cancer in the subject or confirms the diagnosis of a cancer in the subject.

In some cases, the cancer is a ROR1 positive cancer, preferably selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.

In some examples, the control sample is a sample from a subject without cancer. In particular examples, the sample is a blood or tissue sample.

In some embodiments of the methods of diagnosis and detection, the anti-ROR1 antibody is directly labeled with a detectable label. In another embodiment, the anti-ROR1 antibody (the first antibody) is unlabeled and a second antibody or other molecule that can bind the first is labeled. As is well known to one of skill in the art, a secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative embodiment, ROR1 can be assayed in a biological sample by a competition immunoassay utilizing ROR1 protein standards labeled with a detectable substance and an unlabeled anti-ROR1 antibody. In this assay, the biological sample, the labeled ROR1 protein standards and the anti-ROR1 antibody are combined and the amount of labeled ROR1 protein standard bound to the unlabeled antibody is determined. The amount of ROR1 in the biological sample is inversely proportional to the amount of labeled ROR1 protein standard bound to the anti-ROR1 antibody.

The immunoassays and methods disclosed herein can be used for a number of purposes. In one embodiment, the anti-ROR1 antibody may be used to detect the production of ROR1 in cells in cell culture. In another embodiment, the antibody can be used to detect the amount of ROR1 in a biological sample, such as a tumor sample, a tissue sample, or a blood or serum sample. In some examples, the ROR1 is cell-surface ROR1. In other examples, the ROR1 protein is soluble (e.g. in a cell culture supernatant or in a body fluid sample, such as a blood or serum sample).

In one embodiment, a kit is provided for detecting ROR1 in a biological sample, such as a tumor sample, a blood sample or tissue sample. For example, to confirm a cancer diagnosis in a subject, a biopsy can be performed to obtain a tissue sample for histological examination. Kits for detecting a polypeptide will typically comprise a monoclonal anti-ROR1 antibody, such as any of the monoclonal antibodies disclosed herein. In a further embodiment, the antibody is labeled (for example, with a fluorescent, radioactive, or an enzymatic label).

In one embodiment, a kit includes instructional materials disclosing means of use of an anti-ROR1 antibody. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting ROR1 in a biological sample generally includes the steps of contacting the biological sample with an anti-ROR1 antibody. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind ROR1, as disclosed herein, can be used in these assays. Thus, the antibodies can be used in a conventional immunoassay, including, without limitation, an ELISA, an RIA, FACS, tissue immunohistochemistry, Western blot or immunoprecipitation.

### EXAMPLES

### Example 1 Preparation of Antigen and Stable Cell Lines

### 1.1. Preparation of Antigens and Other Proteins

Recombinant human ROR1 ECD Fc-tag protein (Catalog #RO1-H5250) and Human ROR1-ECD-his (abbreviated as hROR1-ECD-his) were purchased from Acro biosystemns (Cat #: RO1-H522y).

### 1.2. Preparation of Stable Cell Lines

HEK293T-hROR1 (human ROR1) was purchased from Kyinno (Cat #: KC-1018). CHO-K1-cynoROR1 (cynomolgus ROR1) and CHO-K1-huROR1 (human ROR1) stable cell lines were prepared as follows. Inoculated CHO-K1 cell suspension with 2 mL/well at a density of 50000/ml into 6-well plates, incubated overnight, then added Polybrene (Shanghai Jikai Gene, REVG0001, 10 mg/ml) to make the final concentration 4 μg/mL, mixed and added 10 μL virus (LV-CynoROR1, Purchased from Shanghai Genechem, 42582-1, titer 1E9/mL, 50 μl/vial), mixed and incubated at 37° C. for 8 hours, then supernatant was discarded and replaced with fresh medium, and cultured at 37° C. After 24 h digestion, the cells were re-suspended in complete medium containing 8 μg/mL puromycin (Thermo, A1113803), cells were diluted to 5/mL and cultured at 37° C. for 10 days with 100 μL/well 10 96-well plates. Monoclones were selected for FACS verification. After verification, the verified clones were further expanded and cultured for freezing storage.

### Example 2 Animal Immunization Schemes

In order to obtain ROR1-specific antibodies, Harbour HCAb transgenic mice (https://harbourantibodies.com/) were immunised through different approaches. These immunisations yielded a number of HCAb antibodies that bind ROR1 extracellular (ECD) proteins or ROR1-expressing cells.

### 2.1 Immunization by Injection of ROR1 Proteins

Recombinant human ROR1 ECD Fc-tag protein (Acro Biosystem, Catalog #RO1-H5250) was used as the immunogen to immunize Harbour HCAb transgenic mice.

The immunization scheme for Harbour HCAb mice immunization cohorts is listed in Table 5 below. In brief, each mouse was administrated with 50 μg of the immunogen for the first boost via i.p. with adjuvant (Sigma, F5881), and 25 μg for following boosts via i.p. with adjuvant (Sigma, S6322). The immunization was conducted bi-weekly for a total of 5 times. Final immunization was conducted with immunogen diluted in PBS via i.p. Serum titers were tested against human recombinant human ROR1 ECD His-tag protein (Acro Biosystem, Catalog #RO1-H522y) using ELISA and against ROR1-expressing cell line using FACS.

TABLE 5
Immunization scheme

| | Animal | | | | Dosage |
|---|---|---|---|---|---|
| Immunogen | No. | Strain | Route | Adjuvant | (ug/animal) |
| huROR1- | 10 | HCAb | i.p. | CFA/Ribi/Ribi/ | 50/25/25/ |
| ECD-huFc | | 2.1 | (250 ul) | Ribi/Ribi | 25/25 ug |

### 2.2 Immunization by Injection of ROR1 Cells

HEK293T-hROR1 was used as the immunogen to immunize Harbour HCAb transgenic mice. The immunization scheme for Harbour HCAb mice immunization cohorts is listed in Table 6 below.

In brief, each mouse was intraperitoneally (i.p.) injected with 1.0×10⁷ HEK293T-hROR1 cells resuspended in PBS for primary immunization. For booster immunization, 1.0×10⁷ HEK293T-hROR1 cells were resuspended in PBS and intraperitoneally injected into mice. The interval between the primary immunization and the first booster immunization was 2 weeks. For the following booster immunization 1.0×10⁷ HEK293T-hROR1 cells were intraperitoneally injected into mice every three weeks with a total of 5 times. Seven days after each booster immunization, the blood was taken, and serum titers were tested against recombinant human ROR1 ECD His-tag protein (Acro Biosystem, Catalog #RO1-H522y) using ELISA and against ROR1-expressing cell line using FACS.

TABLE 6
Immunization scheme

| | Animal | | | | Dosage |
|---|---|---|---|---|---|
| Immunogen | No. | Strain | Route | Adjuvant | (cells/animal) |
| HEK293T- | 10 | HCAb | i.p. | — | 1.0 × 10\hat{ }7 cells |
| huROR1 | | 2.1 | (250 ul) | | |

### Example 3 Screening for ROR1-Specific HCAb Antibodies

### 3.1 HEK293-pCAG-HCAb Directed Cloning Screening for HCAb Antibodies

In this example, lymph nodes from mice with high antibody titers were harvested to prepare cDNA. The variable regions of HCAb cDNA were amplified by PCR using specific primers (5′-GGTGTCCAGTGTSAGGTGCAGCTG-3′ (SEQ ID NO: 262), 5′-AATCCCTGGGCACTGAAGAGACGGTGACC-3′ (SEQ ID NO: 263)) and cloned on mammalian expression vector (pCAG) which contains human immunoglobulin heavy chain Fe part of the IgG1 subclass, named as pCAG-HCAb libraries. The plasmids of pCAG-HCAb libraries were prepared and transfected into HEK293 cells (ATCC, CRL-1573) on 96-well plates for expression, then the supernatants of HEK293-pCAG-HCAb were harvested and transferred to different 96-well plates for screening by in vitro binding assay. Binding to stable cell line CHO-K1-huROR1 expressing human ROR1, and to stable cell line CHO-K1-cynoROR1 expressing cynomolgus monkey ROR1 were tested by Mirrorball (SPT Labtech). HEK293 cell supernatants which exhibited binding to both CHO-K1-huROR1 and CHO-K1-cynoROR1 were selected for subsequently FACS screening.

Finally, multiple HCAb clones were selected for the further characterization.

### 3.2 Single B-Cell Screening for HCAb Antibodies

The Beacon® Optofluidic system was used for single B cell screening. The system uses optical-electric positioning (OEP™) technology to move individual cells, and allow simultaneous biological function tests, experimental analysis, positive clone selection and other operations under cell culture conditions. The Beacon platform can perform these tasks in a massively parallel, automated manner on thousands of cells.

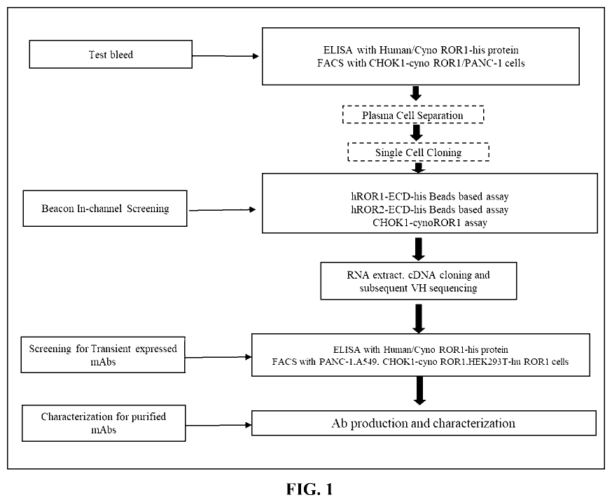

In this example, a plasma cell discovery workflow was used. In each experiment, up to 14,000 individual plasma cells were screened for secretion of ROR1-specific antibodies. Then, plasma cells that secreted antigen-specific antibodies were transferred to 96-well plates for subsequent single B cell sequencing to identify the heavy chain of the antibody produced by a single B cell (monoclonal). FIG. 1 shows the screening strategy and process.

The example used a single B cell sequencing method to obtain the sequences of heavy chain of the antibody from a single plasma cell. General procedures include extraction and purification of the total RNA from single plasma cell lysate, reverse transcription synthesis of cDNA, amplification and purification of cDNA, amplification of the DNA sequences encoding heavy chain of an antibody, cloning and transfection, and Sanger sequencing. Uniqueness and cluster analysis on the obtained sequences was performed, and then DNA sequences encoding the heavy chain of the antibody were synthesized.

### Example 4. Antibody Production and Purification

The recombinant plasmids encoding target antibodies were transiently transfected into HEK293-6E cells (National Research Council) using PEI (Polyscience, 24885). After transfection, the cells were incubated at 37° C. with 5% CO₂ and shaking at 120 rpm. The cell culture supernatants containing target antibodies were harvested 6-7 days post transfection by centrifugation and filtration. Monoclonal antibodies were purified using Protein A magnetic beads (AmMag Protein A Magnetic Beads, Genscript, L00695).

The purity of the antibodies was tested by SEC-HPLC (Agilent 1260 Infinity II HPLC with Welch Xtimate SEC-300 Colum, 1×PBS pH 7.4 as mobile phase) and SDS-PAGE (SurePAGE, Bis-Tris, 10×8, 4-12%, 12 wells, Genscript, M00653). Recombinant antibodies were successfully expressed and purified for further characterization.

By Examples 1-4, HCAb antibody PR005337, PR005338, PR005339, PR005340, PR005341, PR005342, PR005343, PR005344, PR005345, PR005346, PR005347, PR005348, PR005349, PR005350, PR005351, PR005352, PR303125, PR303189, PR303191, PR303199, PR303201, PR303145, PR303147, and PR303155 were obtained. The amino acid sequences of these antibodies were listed in Tables 1-3 above.

In the meantime, anti-ROR1 antibody PR000374 was produced following the procedures showed above with sequence information from international patent application No. WO2016/094873 (which is incorporated herein by reference). PR000374 is a rabbit to human ROR1 Ab from Patent WO2016/094873.

### Example 5. Binding Activity of Antibodies

### 5.1 Binding Activity to ROR1 Expressing Cells

Binding of recombinant anti-ROR1 antibodies to human or cynomolgus ROR1-overexpressing cells was tested by flow cytometry. In this example, ROR1-expressing cell lines are HEK293T cell lines that had been transfected to express human ROR1 on the surface (HEK293T-hu ROR1, KYINNO BIOTECHNOLOGY CO., LTD, Catalog #KC-1018), CHO-K1 cell lines that had been transfected to express cynomolgus ROR1 (CHOK1-cyno ROR1), PANC-1 (ATCC, catalog: CRL-1469) or A549 cell lines (Shanghai Institute of Biochemistry and Cell Biology, Chinese Academy of Sciences).

In brief, anti-ROR1 antibodies were serially diluted in staining buffer (PBS containing 2% FBS). Antibody solution was incubated with 1×10⁵ cells at 4° C. for 1 hour. The cells were washed twice with staining buffer (PBS containing 2% FBS), and 100 μL of 1:1000 diluted florescent labeled anti-human IgG antibody (Alexa Fluor 647 AffiniPure Goat Anti-Human IgG Fc, Jackson ImmunoResearch, Catalog 109-605-098 or Alexa Fluor® 488 AffiniPure Goat Anti-Human IgG(H+L), Jackson ImmunoResearch, 109-545-088) was added into each well. After 1-hour incubation at 4° C., cells were washed twice with staining buffer and subjected to flow cytometry. PR000374 (Bench marker 1) and non-relevant IgG isotype control (Crownbio) were used as positive and negative controls, respectively.

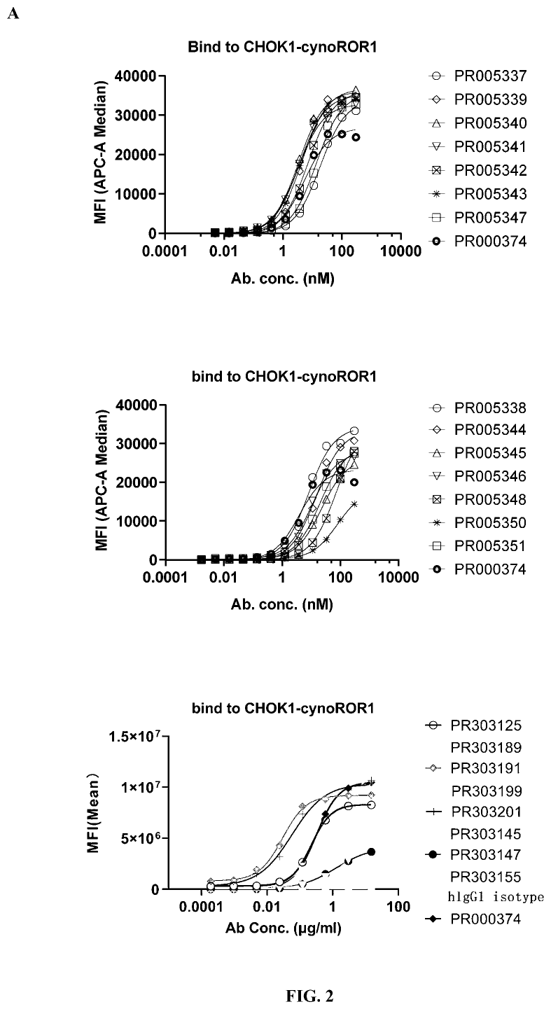

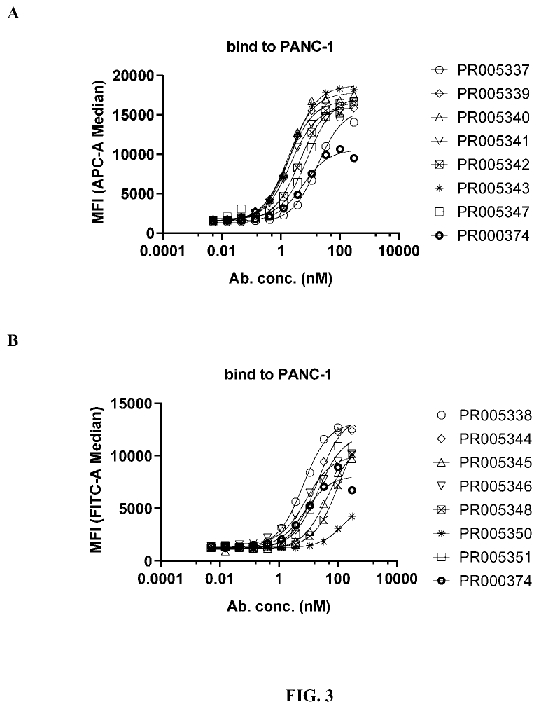

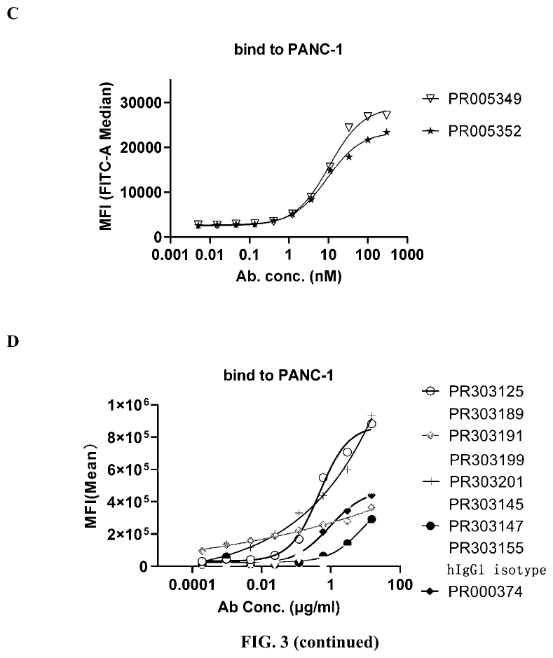

The results for PR005337, PR005338, PR005339, PR005340, PR005341, PR005342, PR005343, PR005344, PR005345, PR005346, PR005347, PR005348, PR005349, PR005350, PR005351, and PR005352 are shown in FIGS. 2A, 2B, 3A, 3B, 3C and Tables 7-8 below. The results showed that the HCAb antibodies had a good binding activity to PANC-1 cells and strong binding activity to CHO-K1-cynoROR1 cells, indicating that these HCAb antibodies had cross-reactivity with cynoROR1.

TABLE 7
Binding of anti-ROR1 HCAb antibodies
to CHO-K1-cyno ROR1 cells

| Antibody ID | EC50 | Max |
|---|---|---|
| PR005337 | 18.18 | 31123 |
| PR005339 | 4.40 | 34835 |
| PR005340 | 3.44 | 36398 |
| PR005341 | 3.22 | 32905 |
| PR005342 | 7.27 | 34495 |
| PR005343 | 3.62 | 34069 |
| PR005347 | 13.49 | 32745 |
| PR000374 | 5.20 | 25195 |
| PR005338 | 8.042 | 33279 |
| PR005344 | 15.09 | 30737 |
| PR005345 | 22.37 | 24571 |
| PR005346 | 10.38 | 26634 |
| PR005348 | 73.37 | 27989 |
| PR005350 | 79.08 | 14394 |
| PR005351 | 20.1 | 27464 |
| PR000374 | 3.883 | 23157 |

TABLE 8
Binding of anti-ROR1 HCAb antibodies to PANC-1 cells

| Antibody ID | EC50 | Max |
|---|---|---|
| PR005337 | 17.10 | 14706 |
| PR005339 | 1.75 | 16194 |
| PR005340 | 1.81 | 17527 |
| PR005341 | 2.13 | 16531 |
| PR005342 | 4.30 | 16681 |
| PR005343 | 2.24 | 18287 |
| PR005347 | 8.08 | 16428 |
| PR000374 | 5.47 | 10681 |
| PR005338 | 6.70 | 12711 |
| PR005344 | 20.01 | 12390 |
| PR005345 | 45.04 | 9714 |
| PR005346 | 8.28 | 10167 |
| PR005348 | 112.80 | 10198 |
| PR005350 | 190.30 | 4248 |
| PR005351 | 20.95 | 10899 |
| PR000374 | 7.51 | 8935 |
| PR005349 | 10.5 | 27100 |
| PR005352 | 9.297 | 23316 |

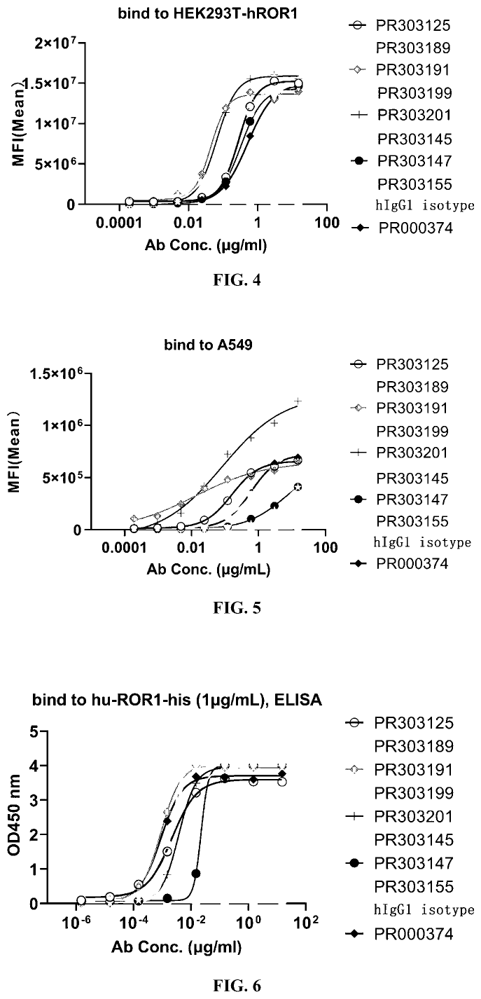

The results for HCAb antibodies PR303125, PR303189, PR303191, PR303199, PR303201, PR303145, PR303 147, and PR303155 are shown in FIGS. 2C, 3D, 4, 5 and Table 9 below. The results indicate that HCAb antibodies showed strong binding activity to both human and cynomolgus ROR1 expressing cells. These results indicate that the anti-ROR1 HCAb antibodies are capable of binding to human and cynomolgus ROR1 on cell membrane with high affinity.

TABLE 9
Binding of anti-ROR1 HCAb antibodies to cell surface ROR1 by FACS

| | HEK293T-hROR1 | | CHO-K1-cynoROR1 | | PANC-1 | | A549 | |
|---|---|---|---|---|---|---|---|---|
| Antibody | EC50 | | EC50 | | EC50 | | EC50 | |
| ID | (μg/ml) | MAX(MFI) | (μg/ml) | MAX(MFI) | (μg/ml) | MAX(MFI) | (μg/ml) | MAX(MFI) |
| PR303125 | 0.273 | 1.50E+07 | 0.222 | 8.27E+06 | 0.463 | 8.83E+05 | 0.16 | 6.68E+05 |
| PR303189 | 0.085 | 1.46E+07 | 7.525 | 5.30E+06 | +/− | 2.40E+05 | — | 9.01E+03 |
| PR303191 | 0.044 | 1.40E+07 | 0.030 | 9.24E+06 | +/− | 3.64E+05 | 0.01 | 6.50E+05 |
| PR303199 | 0.191 | 1.53E+07 | 1.427 | 6.85E+06 | 11.320 | 4.62E+05 | 6.79 | 4.08E+05 |
| PR303201 | 0.066 | 1.55E+07 | 0.057 | 1.07E+07 | +/− | 9.35E+05 | 0.08 | 1.23E+06 |
| PR303145 | 0.158 | 1.35E+07 | 0.474 | 1.04E+07 | 0.654 | 6.53E+05 | 0.93 | 8.75E+05 |
| PR303147 | 0.325 | 1.46E+07 | 1.257 | 3.67E+06 | 7.773 | 2.91E+05 | 6.46 | 4.12E+05 |
| PR303155 | 0.267 | 1.49E+07 | 0.804 | 1.10E+07 | 1.069 | 5.52E+05 | 1.32 | 7.91E+05 |
| Isotype | — | — | — | — | — | — | — | — |
| PR000374 | 0.506 | 1.49E+07 | 0.314 | 1.05E+07 | 0.905 | 4.42E+05 | 0.61 | 6.95E+05 |

5.2 Binding Activity to hu-ROR1-ECD-his

Binding of recombinant anti-ROR1 antibodies to hu-ROR1-ECD-his was tested by ELISA. In brief, 1 μg/mL hu-ROR1-ECD-his was added to the 96-well plate, 100 μL each well, incubated at 4° C. overnight, washing the plates with 1×PBST 3 times, 300 ul/well, blocking the plates with 2% BSA in 1×PBST, 200 ul/well at 37° C. for 1 hour, anti-ROR1 antibodies were serially diluted in staining buffer (PBS containing 2% BSA). Antibody solution was added to the plate and incubates at 37° C. for 1 hour, washing the plates with 1×PBST 3 times, 300 ul/well, adding secondary antibody 100 ul/well and incubate at 37° C. for 1 hour, washing the plates with 1×PBST 3 times, 300 ul/well and then add 100 ul/well TMB for about 5 min, stop the reaction by adding 100 ul/well 2M H₂SO₄, read the plates with Molecular device spectra max plus384 at 450 nm and 570 nm.

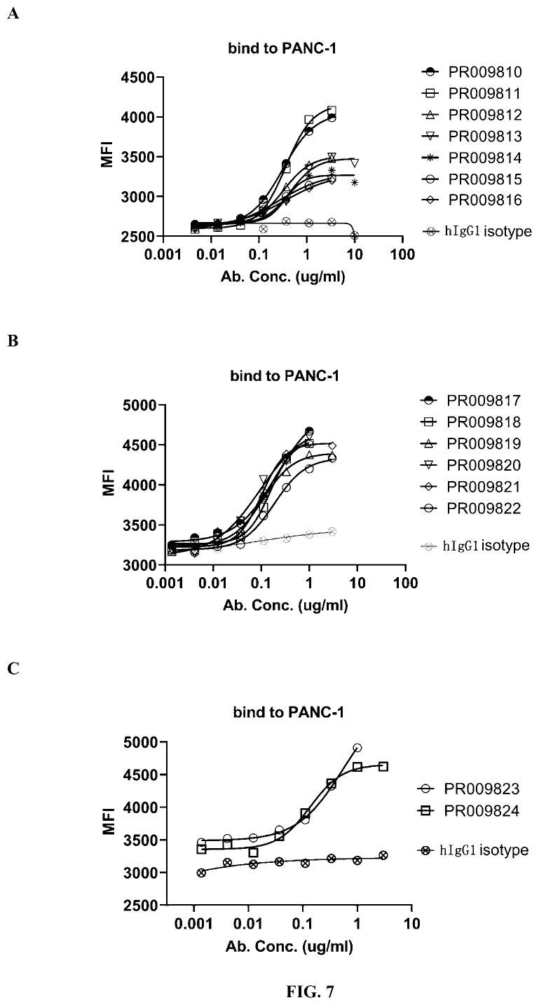

The results for HCAb antibodies are shown in FIG. 6 and Table 10 below. The results indicate that HCAb antibodies showed strong binding activity to hu-ROR1-ECD-his. These results indicate that the anti-ROR1 HCAb antibodies are capable of binding to human ROR1 with high affinity.

TABLE 10
Binding of anti-ROR1 HCAb antibodies
to hu-ROR1-ECD-his by ELISA

| Antibody ID | EC50 (μg/ml) |
|---|---|
| PR303125 | 0.002 |
| PR303189 | 0.002 |
| PR303191 | 0.001 |
| PR303199 | 0.006 |
| PR303201 | 0.004 |
| PR303145 | 0.002 |
| PR303147 | 0.023 |
| PR303155 | 0.002 |
| Isotype | X |
| PR000374 | 0.001 |

### Example 6 PR005340 Affinity Maturation

The affinity maturation of PR005340 was conducted by Yeast surface display with BD FACS AriaIII sorting machine. Firstly, the sequence of PR005340 was analyzed by Kabat numbering and the CDRs regions were also defined. Site saturation and CDR walking strategies were applied for this HCAb affinity maturation. Four mutagenesis libraries were constructed and named as 5340-H1L, 5340-H2L, 5340-H3L and 5340-H2WL, respectively. Two rounds of sorting and screening were performed as follows.

In the 1st round sorting and screening, 5340-H1L, 5340-H2L and 5340-H3L were sorted and screened by BD FACS AriII; the 5340-H2WL library was subjected to MACS enrichment and FACS sorting and screening. For each library, the populations with high binding were gated and sorted out; then sorted yeast cells were also cultured and picked out for sequencing and analysis; the unique hits were also characterized by FACS. Based on the FACS result, several hotspots were selected and combined to a combo mutagenesis library.

In the 2nd round, the combo library was designed and constructed. The library was also subjected to gating and sorting; sorted populations with high binding were also cultured and picked out for sequencing and analysis, all unique hits were characterized by FACS.

Finally, 15 monovalent form (VH-Flag-His) variants (PR009810, PR009811, P009812, PR009813, PR009814, PR009815, PR009816, PR009817, PR009818, PR009819, PR009820, PR009821, PR009822, PR009823, and PR009824) and 17 bivalent form (HCAb) variants (PR007408, PR007409, PR007410, PR007411, PR007412, PR007413, PR007414, PR007415, PR007416, PR007417, R007418, PR007419, PR007420, PR007421, PR007422, PR007423, and PR007424) were screened and synthesized. The amino acid sequences of these variants were listed in Tables 1-3 above.

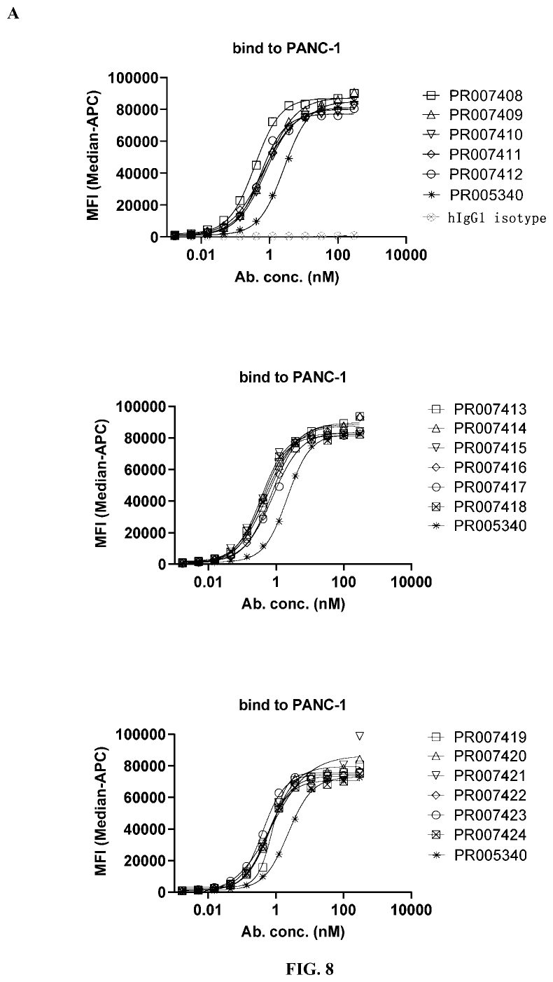

Binding activity of the variants to PANC-1 cells were tested using the same method described in example 5.1. The results were shown in FIGS. 7-8 and Table 11 and Table 12. As shown, most of the variants showed significant binding enhancement compared to PR005340.

TABLE 11
Binding of PR005340 monovalent form variants to PANC-1 cells

| Antibody ID | EC50 | Max |
|---|---|---|
| PR009810 | 0.3132 | 3988 |
| PR009811 | 0.3651 | 4082 |
| PR009812 | 0.3235 | 3481 |
| PR009813 | 0.4616 | 3493 |
| PR009814 | 0.3595 | 3330 |
| PR009815 | 0.2569 | 3232 |
| PR009816 | 0.3707 | 3201 |
| PR009817 | 0.2067 | 4672 |
| PR009818 | 0.1537 | 4518 |
| PR009819 | 0.1083 | 4376 |
| PR009820 | 0.09228 | 4600 |
| PR009821 | 0.1013 | 4524 |
| PR009822 | 0.1861 | 4335 |
| PR009823 | 1.026 | 4909 |
| PR009824 | 1.461 | 4622 |

TABLE 12
Binding of PR005340 bivalent form variants to PANC-1 cells

FACS BINDING TO PANC-1

| ANTIBODY ID | TOP | EC50 |
|---|---|---|
| PR005340 | 84867 | 2.65 |
| PR007408 | 87015 | 0.37 |
| PR007409 | 87438 | 0.8 |
| PR007410 | 80302 | 0.76 |
| PR007411 | 81434 | 0.65 |
| PR007412 | 77181 | 0.55 |
| PR007413 | 89837 | 0.584 |
| PR007414 | 88874 | 0.496 |
| PR007415 | 82756 | 0.3652 |
| PR007416 | 87637 | 0.7235 |
| PR007417 | 83758 | 0.7836 |
| PR007418 | 81348 | 0.3941 |
| PR007419 | 75713 | 0.7909 |
| PR007420 | 79539 | 0.7015 |
| PR007421 | 86578 | 0.7776 |
| PR007422 | 73634 | 0.5903 |
| PR007423 | 74531 | 0.4087 |
| PR007424 | 70766 | 0.5504 |

### Example 7 Binding Activity of Antibodies to Soluble ROR1 Protein by BLI Method

In the example, binding kinetics of anti-ROR1 antibodies to soluble ROR1 protein were analyzed by using Bio-Layer Interferometry (BLI) analysis on Fortebio Octet Red384 instrument (Fortebio).

### 7.1 Binding Affinity of Antibodies to ROR1 Protein by Using HIS1K Sensor

In BLI analysis, recombinant anti-ROR1 antibodies was serially diluted with 10× kinetics buffer (Fortebio). Human ROR1-His proteins were diluted to 20 nM. Then the diluted antibodies, ROR1 proteins and regeneration buffer (10 mM glycine HCl pH 1.5) were added to 96-well plates (Greiner). Rate constants for association and dissociation were measured using HIS1K sensor (Fortebio). The sensor surface was regenerated after each binding experiment with regeneration buffer. The traces were processed using Octet Data Analysis Software (version 11.0, Pall ForteBio, CA, USA).

The binding kinetics parameters for anti-ROR1 HCAb antibodies binding to human ROR1 are summarized in Table 13. As shown in Table 13, in Octect analysis, the HCAb antibodies showed high binding affinity to soluble ROR1.

TABLE 13
Binding of anti-ROR1 HCAb antibodies to soluble human ROR1

| Antibody ID | K_{D} (M) | kon(1/Ms) | kdis(1/s) | Full R\hat{ }2 |
|---|---|---|---|---|
| PR303125 | <1.0E−12 | 1.65E+05 | <1.0E−07 | 0.64 |
| PR303189 | 6.234E−10 | 6.46E+06 | 4.03E−03 | 0.83 |
| PR303191 | 3.95E−11 | 2.90E+06 | 1.15E−04 | 0.98 |
| PR303199 | 1.409E−08 | 1.23E+05 | 1.73E−03 | 0.90 |
| PR303201 | 4.891E−09 | 1.28E+06 | 6.26E−03 | 0.98 |
| PR303145 | 1.11E−08 | 2.78E+05 | 3.10E−03 | 0.99 |
| PR303147 | 1.12E−08 | 1.47E+06 | 1.65E−02 | 0.70 |
| PR303155 | 6.83E−09 | 4.92E+05 | 3.36E−03 | 0.99 |

### 7.2 Binding Affinity of Antibodies to ROR1 Protein by Using AHC Sensor

In BLI analysis, Antibodies were diluted to 5 μg/mL using freshly prepared 1× kinetic buffer (10× kinetic buffer (ForteBio, #18-1105) was diluted with PBS (BBI Life Sciences, #E607016-0500)) and captured on the surface of anti-human Fc (AHC) Octet biosensors (ForteBio, #18-5060) to reach capture levels between 0.6-1.0 nm. The captured biosensors were then dipped in wells containing 2-fold serial dilutions of antigen proteins to detect association signals, followed by dissociation steps in wells containing 1× kinetic buffer. Human ROR1 hits-tagged protein (Acrobiosystems, #RO1-H522y) was diluted from 80 nM to 5 nM; the association phase was 180 seconds, and the dissociation phase was 600 seconds. The sensorgrams were recorded and the reference signals were subtracted before curve fitting using ForteBio Data Analysis 11.0 software. Association rates (kₒₙ) and dissociation rates (k_{dis}) were calculated using a simple one-to-one Langmuir binding model. The equilibrium dissociation constant (K_{D}) was calculated as the ratio of k_{dis}/kₒₙ. The binding kinetics parameters for anti-ROR1 HCAb antibodies binding to human ROR1 are summarized in Table 14.

TABLE 14
Binding of anti-ROR1 antibodies to soluble human ROR1

| | | Antigen | | | | |
|---|---|---|---|---|---|---|
| Antibody | | Concentration | | | | |
| ID | Antigen | (nM) | KD (M) | kon(1/Ms) | kdis(1/s) | Full R\hat{ }2 |
| PR005338 | Human | 5-80 | 2.40E−09 | 1.32E+05 | 3.16E−04 | 0.9954 |
| PR005340 | ROR1, His | 5-80 | 1.20E−09 | 1.43E+05 | 1.71E−04 | 0.9968 |
| PR007417 | Tag | 5-80 | 1.80E−10 | 2.71E+05 | 4.86E−05 | 0.9952 |
| PR007424 | | 5-80 | 1.01E−09 | 2.81E+05 | 2.84E−04 | 0.9955 |

### Example 8. Antibody Specificity Validation

### 8.1 Binding of Antibodies to Human ROR2 Tested by ELISA.

1 μg/mL hu-ROR2-ECD-his (Acrobiocystems, RO2-H52E5) was added to the 96-well plate, 100 μL each well, incubated at 4° C. overnight, washing the plates with 1×PBST 3 times, 300 ul/well, blocking the plates with 2% BSA in 1×PBST, 200 ul/well at 37° C. for 1 hour, anti-ROR1 antibodies were serially diluted in staining buffer (PBS containing 2% BSA). Antibody solution was added to the plate and incubates at 37° C. for 1 hour, washing the plates with 1×PBST 3 times, 300 ul/well, adding secondary antibody 100 ul/well and incubate at 37° C. for 1 hour, washing the plates with 1×PBST 3 times, 300 ul/well and then add 100 ul/well TMB for about 5 min, stop the reaction by adding 100 ul/well 2M H₂SO₄, read the plates with Molecular device spectra max plus384 at 450 nm and 570 nm.

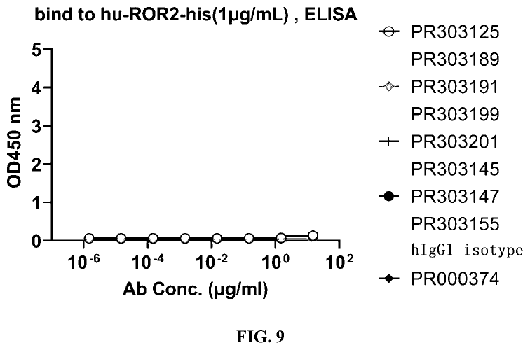

The results for HCAb antibodies are shown in FIG. 9. The results indicate that HCAb antibodies showed no binding activity to hu-ROR2-ECD-his. These results indicate that the anti-ROR1 HCAb antibodies specifically binds to ROR1.

### 8.2 Binding of Antibodies to Human ROR2 Tested by BLI Method

In the example, binding kinetics of anti-ROR1 antibodies to soluble ROR2 protein were analyzed by using Bio-Layer Interferometry (BLI) analysis on Fortebio Octet Red384 instrument (Fortebio). In BLI analysis, Antibodies were diluted to 5 μg/mL using freshly prepared 1× kinetic buffer (10× kinetic buffer (ForteBio, #18-1105) was diluted with PBS (BBI Life Sciences, #E607016-0500)) and captured on the surface of anti-human Fc (AHC) Octet biosensors (ForteBio, #18-5060) to reach capture levels between 0.6-1.0 nm. The captured biosensors were then dipped in wells containing 2-fold serial dilutions of antigen proteins to detect association signals, followed by dissociation steps in wells containing 1× kinetic buffer. Human ROR2 his-tagged protein (Acrobiosystems, #RO2-H52E5) was diluted from 600 nM to 37.5 nM; the association phase was 180 seconds, and the dissociation phase was 180 seconds. The sensorgrams were recorded and the reference signals were subtracted before curve fitting using ForteBio Data Analysis 11.0 software. Association rates (kₒₙ) and dissociation rates (k_{dis}) were calculated using a simple one-to-one Langmuir binding model. The equilibrium dissociation constant (K_{D}) was calculated as the ratio of k_{dis}/kₒₙ.

The binding kinetics parameters for anti-ROR1 HCAb antibodies binding to human ROR2 are summarized in Table 15. The results show that HCAb antibodies have no binding activity to ROR2, suggesting that the anti-ROR1 HCAb antibodies specifically bind to ROR1.

TABLE 15
Binding of anti-RORI antibodies to soluble human ROR2

| | | | | | | |
|---|---|---|---|---|---|---|
| | | Antigen | | | | |
| | | Concentration | KD | kon | kdis | Full |
| Antibody ID | Antigen | (nM) | (M) | (1/Ms) | (1/s) | R\hat{ }2 |

| | | | |
|---|---|---|---|
| PR005338 | Human | 37.5-600 | No binding |
| PR005340 | ROR2, | 37.5-600 | No binding |
| PR007417 | His Tag | 37.5-600 | No binding |
| PR007424 | | 37.5-600 | No binding |

### Example 9. Epitope Binning of Antibodies by Competition Assay

To determine if anti-ROR1 antibodies bind to human ROR1 on different or approximate binding epitopes, the ForteBio Octet® RED96e platform was used to perform epitope competition experiments on the anti-ROR1 antibodies. Human ROR1 protein (Acrobiosystems, #RO1-H522y) was biotinylated and then captured onto SA biosensors (ForteBio, #18-5020) to reach loading level of 0.3-0.4 nm. The in-tandem competition assay format was applied and it contains two association steps. Firstly, the antigen-loaded biosensors bind to each antibody (a.k.a, First antibody, 1^{st} Ab) with a saturating concentration of 200 nM for 180 seconds to reach equilibrium and then secondly bind to the competing antibodies (a.k.a, Second antibody, 2^{nd} Ab) of 200 nM for 180 seconds. The second binding signals were recorded as the 100% signal of each antibody when the first antibodies were replaced by kinetics buffer. All the binding data were analyzed using ForteBio Data Analysis 11.0 software. The inhibition rate is calculated by the following formula:

Inhibition rate (%)=(A−B)/A*100
A: 100% signal of each antibody;
B: the signals of second antibody binding steps.

If the obtained inhibition rate is greater than 80(%), it indicates that the epitopes of the two antibodies completely overlap; if the inhibition rate is less than 40(%), it indicates that the epitopes of the two antibodies are different or far away from each other.

As shown in Tables 16 and 17, HCAb antibodies PR005338, PR005340, PR007417 and PR007424 shared almost identical binding epitopes on human ROR1, but completely different to that of reference antibody PR000374. HCAb antibodies PR303189, PR303199, PR303145, PR303147, PR303155, PR303125, PR303191, PR303201 showed different binding epitopes to that of PR000374; among them, PR303125. PR303191, PR303201 shared similar or overlapped epitopes, but different to other HCAb antibodies.

TABLE 16
The inhibition rates of antibodies in the epitope competition assay

| | | 2^{nd} Ab | | | | |
|---|---|---|---|---|---|---|
| Inhibition (%) | | PR005338 | PR005340 | PR007417 | PR007424 | PR000374 |
| 1^{st} Ab | PR005338 | 103.62% | 107.73% | 107.80% | 102.42% | −3.02% |
| | PR005340 | 106.23% | 109.38% | 108.72% | 106.11% | −0.17% |
| | PR007417 | 113.69% | 116.43% | 112.59% | 111.34% | 10.25% |
| | PR007424 | 107.56% | 111.11% | 111.27% | 106.82% | −0.73% |
| | PR000374 | −7.76% | −7.48% | −10.49% | −8.53% | 111.00% |

TABLE 17
The inhibition rates of antibodies in the epitope competition assay

| Inhibition (%) | PR303189 | PR303199 | PR303145 | PR303147 | PR303155 | PR303125 | PR303191 | PR303201 | PR000374 |
|---|---|---|---|---|---|---|---|---|---|
| PR303191 | −99% | −57% | −70% | −60% | −62% | 80% | 80% | 57% | −38% |
| PR303125 | −24% | −48% | −41% | −88% | −62% | 104% | 109% | 86% | −24% |
| PR000374 | −52% | −61% | −61% | −72% | −67% | −45% | −34% | −63% | 109% |

### Example 10. Antibody Internalization by ROR1 Expressing Cells

### 10.1 Antibody Internalization by HEK293T-hROR1 Cells by Ab-MMAF Cytotoxicity Method

12000 cells/90 ul of HEK293T-hROR1 cells were added to flat bottom 96-well plates, incubated overnight in 37° C., 5% CO2 incubator. Antibodies at 10× concentration (100 nM) in complete medium were prepared. The dilution factor is 5 and 6 doses (10, 2, 0.4, 0.08, 0.016, 0.0032) were prepared. 10 ul of each complex dilution was transferred to the cells in duplicate to final volume of 100 ul for all assay well. aHFc-CL-MMAF at concentration of 50 ug/ml (50×) was prepared and added with 2 ul to the wells, the final concentration is 1 ug/mil. Cells were incubated for 120 h at 37° C. 5% CO₂. Add 100 ul of Cell Titer-Glo Reagent equal to the volume of cell culture medium present in each well. Contents were mixed for 2 minutes on an orbital shaker to induce cell lysis. The plate was incubated at room temperature for 10 minutes to stabilize the luminescent signal. Luminescence was recorded using PE Enspire.

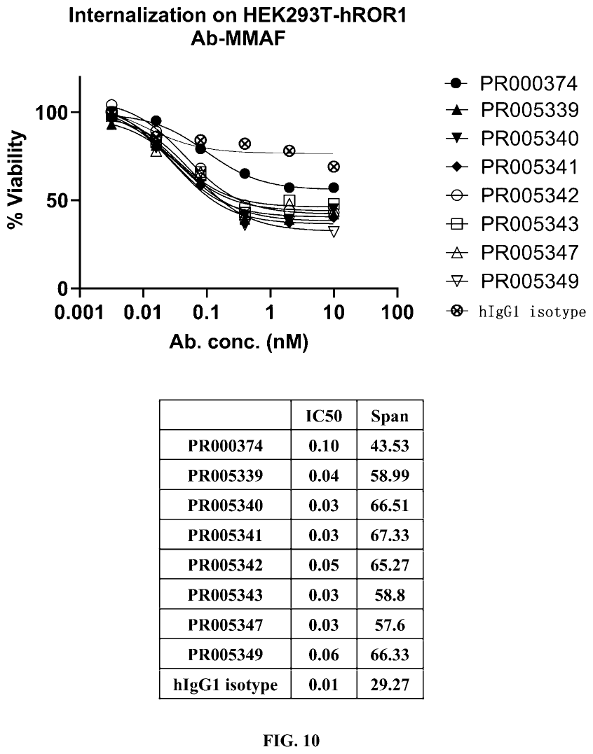

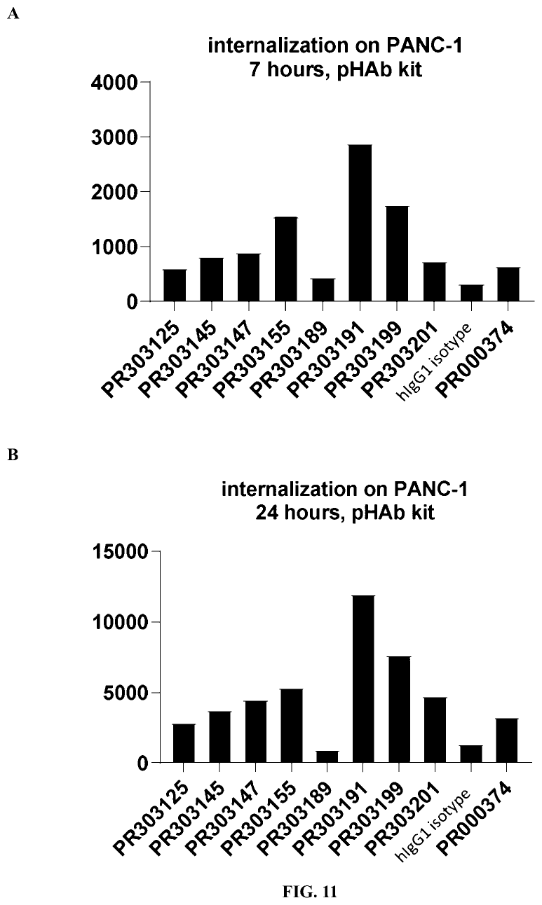

The results of internalization rate of anti-ROR1 HCAbs are shown in FIG. 10. HCAbs showed better internalization activity on ROR1 overexpressing 293T cells than PR000374.

10.2 Antibody Internalization by PANC-1 Cells by pHAb Kit

In this example, pHAb Amine Reactive Dye (Promega, Cat #G9845) was used to determine the antigen-based internalization of anti-ROR1 antibodies into PANC-1 cells. PHAb Dyes are pH sensor dyes that have very low fluorescence at pH>7 and a dramatic increase in fluorescence when the pH of the solution becomes acidic. When an antibody labelled with pHAb dyes binds outside membrane of cells in neutral pH, no or very low fluorescence could be monitored. After internalization, the fluorescence will become stronger in lower pH environments in endosomes and lysosomes.

Antibodies were labelled with pHAb Dyes and calculated for DARs following the kit instructions. The labelled antibodies were then incubated with PANC-1 at 4° C. (the internalization activity at this temperature is very low, which was used as background control) or 37° C. for 24 hours. Then a fluorescence with excitation maxima (Ex) at 532 nm and emission maxima (Em) at 560 nm was detected. The final normalized results are shown as the fluorescence intensity under 37° C. subtracting the fluorescence intensity at background under 4° C. and then divided by DARs of pHAb Dye of the antibody. A higher value indicates a higher internalization activity.

The results of internalization rate of anti-ROR1 HCAb antibodies detected at 7^{th} hour and 24^{th} hour are separately shown in FIGS. 11A-11B. The results indicate that HCAb clones PR303155, PR303191 and PR303199 show good internalization by PANC-1 cells.

1. An antibody that specifically binds to ROR1, or an antigen binding fragment thereof, wherein the antibody comprises a heavy chain variable region (VH), and wherein the VH comprises HCDRs 1-3 of a VH having the amino acid sequence set forth in any one of SEQ ID NOs: 150, 180-194, 163-179, 147-149, 151-162, and 195-202.

2. The antibody or antigen binding fragment thereof according to claim 1, wherein the VH comprises HCDRs 1-3 having the amino acid sequences set forth in:
SEQ ID NOs: 12, 40, 103 respectively,
SEQ ID NOs: 17, 53, 119 respectively,
SEQ ID NOs: 17, 53, 116 respectively,
SEQ ID NOs: 17, 53, 120 respectively,
SEQ ID NOs: 17, 53, 121 respectively,
SEQ ID NOs: 17, 53, 122 respectively,
SEQ ID NOs: 17, 53, 123 respectively,
SEQ ID NOs: 17, 53, 124 respectively,
SEQ ID NOs: 17, 53, 125 respectively,
SEQ ID NOs: 17, 52, 116 respectively,
SEQ ID NOs: 17, 53, 117 respectively,
SEQ ID NOs: 17, 52, 117 respectively,
SEQ ID NOs: 17, 40, 116 respectively,
SEQ ID NOs: 17, 53, 118 respectively,
SEQ ID NOs: 17, 54, 116 respectively,
SEQ ID NOs: 17, 55, 116 respectively,
SEQ ID NOs: 17, 54, 117 respectively,
SEQ ID NOs: 17, 40, 117 respectively,
SEQ ID NOs: 17, 55, 117 respectively,
SEQ ID NOs: 17, 54, 118 respectively,
SEQ ID NOs: 17, 40, 118 respectively,
SEQ ID NOs: 17, 55, 118 respectively,
SEQ ID NOs: 17, 55, 103 respectively,
SEQ ID NOs: 12, 55, 117 respectively,
SEQ ID NOs: 12, 55, 118 respectively,
SEQ ID NOs: 11, 40, 100 respectively,
SEQ ID NOs: 12, 41, 101 respectively,
SEQ ID NOs: 12, 42, 102 respectively,
SEQ ID NOs: 12, 43, 104 respectively,
SEQ ID NOs: 13, 44, 105 respectively,
SEQ ID NOs: 12, 40, 106 respectively,
SEQ ID NOs: 12, 45, 107 respectively,
SEQ ID NOs: 12, 40, 108 respectively,
SEQ ID NOs: 14, 46, 109 respectively,
SEQ ID NOs: 12, 47, 110 respectively,
SEQ ID NOs: 12, 48, 111 respectively,
SEQ ID NOs: 12, 45, 112 respectively,
SEQ ID NOs: 15, 49, 113 respectively,
SEQ ID NOs: 16, 50, 114 respectively,
SEQ ID NOs: 12, 51, 115 respectively,
SEQ ID NOs: 18, 56, 126 respectively,
SEQ ID NOs: 21, 40, 130 respectively,
SEQ ID NOs: 22, 60, 131 respectively,
SEQ ID NOs: 12, 40, 132 respectively,
SEQ ID NOs: 23, 61, 133 respectively,
SEQ ID NOs: 19, 57, 127 respectively,
SEQ ID NOs: 20, 58, 128 respectively, or
SEQ ID NOs: 14, 59, 129 respectively.

3. The antibody or antigen binding fragment thereof according to claim 1, wherein the VH comprises an amino acid sequence having at least 80%, at least 85%, at least 90%, at least 95%, at least 98%, at least 99%, or 100% sequence identity to any one of SEQ ID NOs: 150, 180-194, 163-179, 147-149, 151-162, and 195-202.

4. (canceled)

5. The antibody or antigen binding fragment thereof according to claim 1, wherein the antibody comprises a heavy chain (HC), and wherein the HC comprises an amino acid sequence having at least 80%, at least 85%, at least 90%, at least 95%, at least 98%, at least 99%, or 100% sequence identity to any one of SEQ ID NOs: 208, 238-252, 221-237, 205-207, 209-220, and 253-260.

6. The antibody or antigen binding fragment thereof according to claim 1, wherein the antibody does not comprise a light chain.

7. (canceled)

8. The antibody or antigen binding fragment thereof of claim 1, wherein the antibody is a chimeric antibody, a humanized antibody, or a human antibody.

9. The antibody or the antigen binding fragment thereof according to claim 1, wherein the antibody is of an isotype selected from the group consisting of IgG, IgA, IgM, IgE and IgD.

10. The antibody or the antigen binding fragment thereof according to claim 1, wherein the antibody is of a subtype selected from the group consisting of IgG1, IgG2, IgG3, and IgG4.

11. The antibody or the antigen binding fragment thereof according to claim 1, wherein the antigen binding fragment is selected from the group consisting of HCAb, VHH, nanobody, Fab, Fab′, F(ab′)₂, Fd, Fd′, and dAb.

12. The antibody or the antigen binding fragment thereof according to claim 1, wherein the antibody is a monoclonal antibody, a bi-specific or a multi-specific antibody and/or, the antibody is monovalent, bivalent or multivalent.

13. (canceled)

14. The antibody or antigen binding fragment thereof of claim 1, wherein the antibody or antigen binding fragment is attached to a fluorescent label, radiolabel or cytotoxic agent.

15. A bi-specific antibody, comprising the antibody or antigen-binding fragment thereof according to claim 1 and a second antigen binding region specifically binding to a tumor associated antigen or an immune cell antigen, or binding to CD3.

16. A nucleic acid comprising a nucleotide sequence encoding the antibody or the antigen binding fragment thereof according to claim 1 or a bi-specific antibody comprising the antibody or the antigen binding fragment thereof according to claim 1 and a second antigen binding region specifically binding to a tumor associated antigen or an immune cell antigen.

17. (canceled)

18. (canceled)

19. An antibody-drug conjugate (ADC), comprising the antibody or the antigen binding fragment thereof according claim 1 or a bi-specific antibody comprising the antibody or the antigen binding fragment thereof according to claim 1 and a second antigen binding region specifically binding to a tumor associated antigen or an immune cell antigen.

20. A pharmaceutical composition comprising (i) the antibody or the antigen binding fragment thereof according to claim 11, a bi-specific antibody comprising the antibody or the antigen binding fragment thereof according to claim 1 and a second antigen binding region specifically binding to a tumor associated antigen or an immune cell antigen, or an antibody-drug conjugate comprising the antibody or the antigen binding fragment thereof according to claim 1; and (ii) a pharmaceutically acceptable carrier or excipient.

21. The pharmaceutical composition according to claim 20, wherein the composition further comprises a second therapeutic agent selected from the group consisting of an antibody, a chemotherapeutic agent, a siRNA, an antisense oligonucleotide, a polypeptide, and a small molecule drug.

22. A method of treating a cancer in a subject, comprising administering to the subject an effective amount of the antibody or the antigen binding fragment thereof according to claim 1, a bi-specific antibody comprising the antibody or the antigen binding fragment thereof according to claim 1 and a second antigen binding region specifically binding to a tumor associated antigen or an immune cell antigen, comprising the antibody or the antigen binding fragment thereof according to claim 1, or a pharmaceutical composition comprising the antibody or the antigen binding fragment thereof according to claim 1.

23. The method according to claim 22, wherein the cancer is a ROR1 positive cancer.

24. The method according to claim 22, further comprising administering to the subject a second therapeutic agent, wherein the second therapeutic agent is selected from an antibody, a chemotherapeutic agent, a siRNA, an antisense oligonucleotide, a polypeptide, and a small molecule drug.

25-35. (canceled)

36. The method according to claim 22, wherein the cancer is selected from the group consisting of B-cell leukemia, lymphoma, acute myeloid leukemia (AML), Burkitt lymphoma, mantle cell lymphoma (MCL), acute lymphoblastic leukemia (ALL), chronic lymphoblastic leukemia (CLL), diffuse large B-cell lymphoma (DLBCL), follicular lymphoma (FL), marginal zone lymphoma (MZL), breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer, and adrenal cancer.
